(19) United States
(12) Patent Application Publication
(10) Pub. No.: US 2023/0181527 A1
(43) Pub. Date: Jun. 15, 2023

(54) APICAL SODIUM-DEPENDENT TRANSPORTER INHIBITOR COMPOSITIONS

(71) Applicant: Mirum Pharmaceuticals, Inc., Foster City, CA (US)

(72) Inventors: Thirumala KOMMURU, Lexington, MA (US); Jason Brittain, El Cajon, CA (US); Helen Hwang, Foster City, CA (US); Pamela Vig, Foster City, CA (US)

(73) Assignee: Mirum Pharmaceuticals, Inc., Foster City, CA (US)

(21) Appl. No.: 17/973,194

(22) Filed: Oct. 25, 2022

Related U.S. Application Data
(60) Provisional application No. 63/271,857, filed on Oct. 26, 2021.

(51) Int. Cl. A61K 31/38; A61K 31/554; A61K 31/4985; A61K 45/06; A61P 1/16

(52) U.S. Cl. CPC A61K 31/38; A61K 31/554; A61K 31/4985; A61K 45/06; A61P 1/16

(57) ABSTRACT

Provided herein are pharmaceutical compositions comprising apical sodium-dependent transporter inhibitors (ASBTIs) and methods of using same for treatment of cholestatic liver diseases.

### CROSS-REFERENCE TO RELATED APPLICATIONS

This application claims priority to U.S. Provisional Application No. 63/271,857, filed Oct. 26, 2021, the disclosure of which is herein incorporated by reference in its entirety.

### FIELD OF THE INVENTION

The present invention relates to pharmaceutical compositions comprising apical sodium-dependent transporter inhibitors (ASBTIs) and methods of using same for treatment of cholestatic liver diseases.

### BACKGROUND OF THE INVENTION

Hypercholemia and cholestatic liver diseases are liver diseases associated with impaired bile secretion (i.e., cholestasis), associated with and often secondary to the intracellular accumulation of bile acids/salts in the hepatocyte. Hypercholemia is characterized by increased serum concentration of bile acid or bile salt. Cholestasis can be categorized clinicopathologically into two principal categories of obstructive, often extrahepatic, cholestasis, and nonobstructive, or intrahepatic, cholestasis. Nonobstructive intrahepatic cholestasis can further be classified into two principal subgroups of primary intrahepatic cholestasis that result from constitutively defective bile secretion, and secondary intrahepatic cholestasis that result from hepatocellular injury. Primary intrahepatic cholestasis includes diseases such as benign recurrent intrahepatic cholestasis, which is predominantly an adult form with similar clinical symptoms, and progressive familial intrahepatic cholestasis types 1, 2, and 3, which are diseases that affect children. Neonatal respiratory distress syndrome and lung pneumonia is often associated with intrahepatic cholestasis of pregnancy. Active treatment and prevention is limited. In the past, effective treatments for hypercholemia and cholestatic liver diseases include surgery, liver transplantation, and rarely administration of ursodiol.

Pediatric cholestatic liver diseases affect a small percentage of children, but therapy results in significant healthcare costs each year. Currently, many of the pediatric cholestatic liver diseases require invasive and costly treatments such as liver transplantation and surgery.

It is well understood and accepted that the therapeutic needs of children are sufficiently different than those of adults as to require specific studies of medications in children. For example, oral administration of a solid dosage form of medication is painless and simple for most adult patients, but for the pediatric patient population, swallowing an oral solid dosage form produced for adults can be problematic. In addition, the drugs used in solid dosages often have an unpleasant taste. More importantly, oral administration of adult medication targeting cholestatic liver diseases may result in side effects such as diarrhea and intestinal discomfort. Such problems pose a safety risk and affect compliance. Effective and acceptable forms of pediatric medication for pediatric cholestatic liver diseases are needed.

The apical sodium-dependent transporter (ASBT) protein located in the terminal ileum plays an important physiological role in the enterohepatic circulation of bile acids and therefore essential for the bile acid homeostasis. To this end, pharmacological inhibition of ASBT is fast emerging as an interesting target.

Some ASBT inhibitors (ASBTIs) are designed to limit systemic absorption by the individual. In this regard, in some cases formulating these compounds into stable and effective compositions may be challenging.

Thus, there exists an unmet need for safe and effective formulations and compositions comprising ASBTIs.

### SUMMARY OF THE INVENTION

Various non-limiting aspects and embodiments of the invention are described below.

In one aspect, the present invention provides a pharmaceutical composition comprising an ASBTI, a preservative, and an antioxidant.

In one embodiment, the preservative is an antimicrobial preservative. In one embodiment, the preservative is propylene glycol.

In one embodiment, the preservative is present in an amount of at least 30% of the composition. In one embodiment, the preservative is present in an amount of from about 30% to about 40% of the composition. In one embodiment, the preservative is present in an amount of from about 32% to about 37% of the composition. In one embodiment, the preservative is present in an amount of from about 33% to about 36% of the composition. In one embodiment, the preservative is present in an amount of about 33% of the composition. In one embodiment, the preservative is present in an amount of about 34% of the composition. In one embodiment, the preservative is present in an amount of about 35% of the composition.

In one embodiment, the antioxidant is an aminocarboxylic acid or an aminopolycarboxylic acid. In one embodiment, the antioxidant is an aminopolycarboxylic acid selected from EDTA (ethylenediaminetetraacetic acid), DTPA (diethylenetriaminepentaacetic acid), EGTA (ethylene glycol-bis(β-aminoethyl ether)-N,N,N′,N′-tetraacetic acid), NTA (nitrilotriacetic acid), BAPTA (1,2-bis(o-aminophenoxy)ethane-N,N,N′,N′-tetraacetic acid), NOTA (2,2′,2″-(1,4,7-triazonane-1,4,7-triyl)triacetic acid), DOTA (tetracarboxylic acid), and EDDHA (ethylenediamine-N,N′-bis(2-hydroxyphenylacetic acid). In one embodiment, the antioxidant is EDTA.

In one embodiment, the ASBTI is

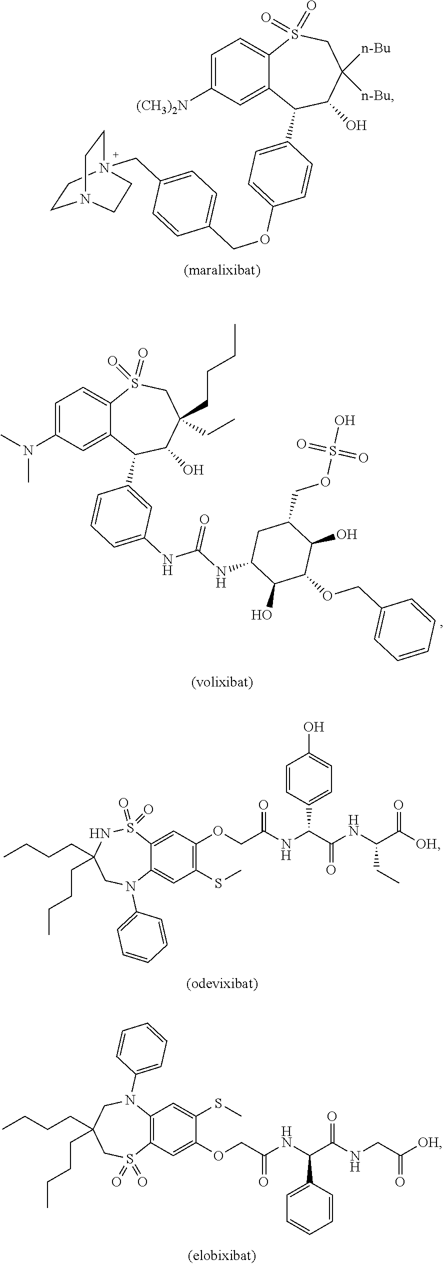

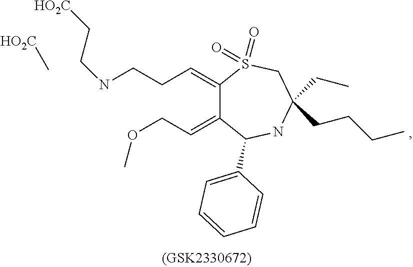

or a pharmaceutically acceptable salt thereof.

In one embodiment, the ASBTI is

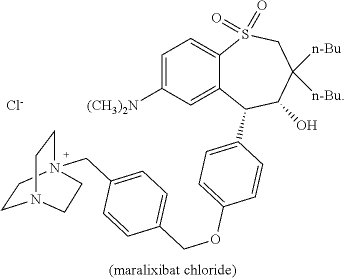

In one embodiment, the ASBTI is volixibat, or a pharmaceutically acceptable salt thereof.

In one embodiment, the ASBTI is odevixibat, or a pharmaceutically acceptable salt thereof.

In one embodiment, the ASBTI is elobixibat, or a pharmaceutically acceptable salt thereof.

In one embodiment, the ASBTI is GSK2330672, or a pharmaceutically acceptable salt thereof.

In one embodiment, the ASBTI is present in an amount of about 0.1 mg/mL to about 500 mg/mL of the composition. In one embodiment, the ASBTI is present in an amount of about 1 mg/mL to about 250 mg/mL of the composition. In one embodiment, the ASBTI is present in an amount of about 2 mg/mL to about 100 mg/mL of the composition. In one embodiment, the ASBTI is present in an amount of about 5 mg/mL to about 50 mg/mL of the composition. In one embodiment, the ASBTI is present in an amount of about 8 mg/mL to about 20 mg/mL of the composition. In one embodiment, the ASBTI is present in an amount of about 9 mg/mL to about 10 mg/mL of the composition. In one embodiment, the ASBTI is present in an amount of about 10 mg/mL of the composition. In one embodiment, the ASBTI is present in an amount of about 9.5 mg/mL of the composition.

In one embodiment, the preservative is an antimicrobial preservative.

In one embodiment, the antimicrobial preservative is selected from the group consisting of propylene glycol, ethyl alcohol, glycerin, benzalkonium chloride, benzethonium chloride, benzoic acid, benzyl alcohol, butylparaben, cetrimide (cetyltrimethylammonium bromide), cetrimonium bromide, cetylpyridinium chloride, chlorhexidine, chlorobutanol, chlorocresol, cresol, ethylparaben, methylparaben, phenol, phenoxyethanol, phenylethyl alcohol, phenylmercuric acetate, phenylmercuric borate, phenylmercuric nitrate, propylparaben, sodium benzoate, sodium dehydroacetate, sodium propionate, sorbic acid, potassium sorbate, thimerosal, thymol, and combinations thereof.

In one embodiment, the preservative is propylene glycol.

In one embodiment, the preservative is present in an amount of at least about 30% w/w of the composition. In one embodiment, the preservative is present in an amount of from about 30% to about 40% of the composition. In one embodiment, the preservative is present in an amount of from about 32% to about 37% of the composition. In one embodiment, the preservative is present in an amount of from about 33% to about 36% of the composition. In one embodiment, the preservative is present in an amount of about 33% of the composition. In one embodiment, the preservative is present in an amount of about 34% of the composition. In one embodiment, the preservative is present in an amount of about 35% of the composition.

In one embodiment, the antioxidant is selected from the group consisting of an aminocarboxylic acid, an aminopolycarboxylic acid, ascorbic acid, ascorbyl palmitate, butylated hydroxyanisole, butylated hydroxytoluene, monothioglycerol, sodium ascorbate, sodium formaldehyde sulfoxylate, sodium metabisulfite, BHT, BHA, sodium bisulfite, vitamin E or a derivative thereof, propyl gallate, and combinations thereof.

In one embodiment, the antioxidant is an aminopolycarboxylic acid selected from EDTA (ethylenediaminetetraacetic acid), DTPA (diethylenetriaminepentaacetic acid), EGTA (ethylene glycol-bis(β-aminoethyl ether)-N,N,N′,N′-tetraacetic acid), NTA (nitrilotriacetic acid), BAPTA (1,2-bis(o-aminophenoxy)ethane-N,N,N′,N′-tetraacetic acid), NOTA (2,2′,2″-(1,4,7-triazonane-1,4,7-triyl)triacetic acid), DOTA (tetracarboxylic acid), and EDDHA (ethylenediamine-N,N′-bis(2-hydroxyphenylacetic acid)

In one embodiment, the antioxidant is EDTA.

In one embodiment, the antioxidant is present in an amount of about 0.001% to about 1% w/w of the composition. In one embodiment, the antioxidant is present in an amount of about 0.005% to about 0.75% w/w of the composition. In one embodiment, the antioxidant is present in an amount of about 0.01% to about 0.5% w/w of the composition. In one embodiment, the antioxidant is present in an amount of about 0.05% to about 0.25% w/w of the composition. In one embodiment, the antioxidant is present in an amount of about 0.075% to about 0.2% w/w of the composition. In one embodiment, the antioxidant is present in an amount of about 0.1% w/w of the composition.

In one embodiment, the composition is stable for at least 1 month at room temperature. In one embodiment, the composition is stable for at least 2 months at room temperature. In one embodiment, the composition is stable for at least 3 months at room temperature. In one embodiment, the composition is stable for at least 6 months at room temperature. In one embodiment, the composition is stable for at least 1 year at room temperature. In one embodiment, the composition is stable for at least 2 years at room temperature.

In one embodiment, the composition is a liquid composition for oral administration. In one embodiment, the composition is an aqueous solution.

In one embodiment, the composition further comprises a sweetener, a taste-masking ingredient, or a combination thereof.

In another aspect, the present invention provides a pharmaceutical composition comprising:
a. from about 5 mg/mL to about 50 mg/mL of maralixibat;
b. from about 300 mg/mL to about 400 mg/mL of propylene glycol;
c. about 1 mg/mL of disodium EDTA;
d. a sweetener, a taste-masking ingredient, or a combination thereof, and
e. water.

In one embodiment, the pharmaceutical composition comprises:
a. from about 8 mg/mL to about 20 mg/mL of maralixibat;
b. from about 330 mg/mL to about 380 mg/mL of propylene glycol;
c. about 1 mg/mL of disodium EDTA;
d. a sweetener, a taste-masking ingredient, or a combination thereof, and
e. water.

In one embodiment, maralixibat is present as maralixibat chloride.

In one embodiment, the pharmaceutical composition further comprises a second therapeutic agent.

In one embodiment, the second therapeutic agent is ursodeoxycholic acid (UDCA), rifampicin, an antihistamine, or an FXR-targeting drug.

In another aspect, the present invention provides a pharmaceutical dosage form for oral administration comprising the pharmaceutical composition of any of the preceding embodiments.

In another aspect, the present invention provides a method of treating or ameliorating a pediatric cholestatic liver disease comprising administering to a pediatric subject a therapeutically effective amount of the pharmaceutical composition or the pharmaceutical dosage form of any of the preceding embodiments.

In one embodiment, the pediatric cholestatic liver disease is progressive familial intrahepatic cholestasis (PFIC), PFIC type 1, PFIC type 2, PFIC type 3, Alagille syndrome (ALGS), biliary atresia (BA), post-Kasai biliary atresia, post-liver transplantation biliary atresia, Dubin-Johnson Syndrome, post-liver transplantation cholestasis, post-liver transplantation associated liver disease, intestinal failure associated liver disease, bile acid mediated liver injury, pediatric primary sclerosing cholangitis (PSC), MRP2 deficiency syndrome, neonatal sclerosing cholangitis, a pediatric obstructive cholestasis, a pediatric non-obstructive cholestasis, a pediatric extrahepatic cholestasis, a pediatric intrahepatic cholestasis, a pediatric primary intrahepatic cholestasis, a pediatric secondary intrahepatic cholestasis, benign recurrent intrahepatic cholestasis (BRIC), BRIC type 1, BRIC type 2, BRIC type 3, total parenteral nutrition associated cholestasis, paraneoplastic cholestasis, Stauffer syndrome, drug-associated cholestasis, infection-associated cholestasis, or gallstone disease.

In one embodiment, the pediatric cholestatic liver disease is PFIC, ALGS, BA, or pediatric PSC.

In one embodiment, the pediatric cholestatic liver disease is characterized by one or more symptoms selected from jaundice, pruritus, cirrhosis, hypercholemia, neonatal respiratory distress syndrome, lung pneumonia, increased serum concentration of bile acids, increased hepatic concentration of bile acids, increased serum concentration of bilirubin, hepatocellular injury, liver scarring, liver failure, hepatomegaly, xanthomas, malabsorption, splenomegaly, diarrhea, pancreatitis, hepatocellular necrosis, giant cell formation, hepatocellular carcinoma, gastrointestinal bleeding, portal hypertension, hearing loss, fatigue, loss of appetite, anorexia, peculiar smell, dark urine, light stools, steatorrhea, failure to thrive, and renal failure.

In another aspect, the present invention provides a method of treating or ameliorating pruritus comprising administering to a pediatric subject a therapeutically effective amount of the pharmaceutical composition or the pharmaceutical dosage form of any of the preceding embodiments.

In another aspect, the present invention provides a method of treating or ameliorating hypercholemia comprising administering to a pediatric subject a therapeutically effective amount of the pharmaceutical composition or the pharmaceutical dosage form of any of the preceding embodiments.

In another aspect, the present invention provides a method of treating or ameliorating xanthoma comprising administering to a pediatric subject a therapeutically effective amount of the pharmaceutical composition or the pharmaceutical dosage form of any of the preceding embodiments.

In another aspect, the present invention provides a method of decreasing the level of serum or hepatic bile levels in a subject comprising administering to a pediatric subject a therapeutically effective amount of the pharmaceutical composition or the pharmaceutical dosage form of any of the preceding embodiments.

In one embodiment of any of the preceding methods, the pediatric subject is between 6 months and 18 years of age.

In one embodiment of any of the preceding methods, the method further comprises administering a second therapeutic agent.

In one embodiment, the second therapeutic agent is UDCA, rifampicin, an antihistamine, an FXR-targeting drug, or a combination thereof.

In one embodiment, the second therapeutic agent is administered in a subclinical therapeutically effective amount.

In yet another aspect, the present invention provides a method of treating or ameliorating a pediatric cholestatic liver disease comprising administering to a pediatric subject a therapeutically effective amount of the pharmaceutical composition or the pharmaceutical dosage form of any of the preceding embodiments in combination with a subclinical therapeutically effective amount of a second therapeutic agent selected from the group consisting of UDCA, rifampicin, an antihistamine, and an FXR-targeting drug.

In one embodiment, the subclinical therapeutically effective amount of the second therapeutic agent is at least 10% lower than the amount of the second therapeutic agent administered as a monotherapy. In one embodiment, the subclinical therapeutically effective amount of the second therapeutic agent is at least 20% than the amount of the second therapeutic agent administered a monotherapy.

In one embodiment, the second therapeutic agent is a PPAR agonist. In one embodiment, the PPAR agonist is selected from bezafibrate, seladelpar (MBX-8025), GW501516 (Cardarine), fenofibrate, elafibranor, REN001, KD3010, ASP0367, and CER-002.

In one embodiment, the PPAR agonist is a PPARδ agonist. In one embodiment, the PPARδ agonist is selected from seladelpar (MBX-8025), REN001, KD3010, ASP0367, and CER-002.

In yet another aspect, the present invention provides a method of treating or ameliorating a pediatric cholestatic liver disease comprising administering to a pediatric subject a therapeutically effective amount of maralixibat in combination with a therapeutically effective amount of a PPAR agonist.

In one embodiment, the PPAR agonist is selected from bezafibrate, seladelpar (MBX-8025), GW501516 (Cardarine), fenofibrate, elafibranor, REN001, KD3010, ASP0367, and CER-002.

In one embodiment, the PPAR agonist is a PPARδ agonist. In one embodiment, the PPARδ agonist is selected from seladelpar (MBX-8025), REN001, KD3010, ASP0367, and CER-002.

The method of claim 69, wherein the pediatric cholestatic liver disease is sclerosing cholangitis.

The method of claim 69, wherein the pediatric cholestatic liver disease is selected from PSC and PBC.

These and other aspects of the present invention will become apparent to those skilled in the art after a reading of the following detailed description of the invention, including the appended claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a plot of the stability of a maralixibat oral solution at 25° C. and 60% relative humidity (RH).

FIG. 2 is a plot of the effect of Disodium EDTA Dihydrate concentration on oxidative impurity desmethyl maralixibat chloride Levels in a maralixibat oral solution at 25° C. and 40° C.

FIGS. 3A-3E are plots of ItchRO and doses of maralixibat and selected antipruritic medications for 5 exemplary PFIC patients enrolled in the LUM001-501 Study. FIG. 3A shows the patient discontinued Rifampicin and UDCA while maintaining excellent pruritus control. FIG. 3B shows the patient discontinued Rifampicin while maintaining excellent pruritus control. FIG. 3C shows the patient discontinued Rifampicin while maintaining excellent pruritus control. FIG. 3D shows the patient discontinued UDCA while maintaining pruritus control. FIG. 3E shows the patient discontinued UDCA while maintaining pruritus control.

FIG. 4 depicts mean liver and serum bile acid, ALT, total bilirubin, and ALP concentrations, displayed in relation to mean values in vehicle treated MDR2−/− mice. One-way-ANOVA was applied to determine differences between treatment groups vs “vehicle control” with ****p<0.0001, ***p<0.001, **p<0.01, *p<0.05.

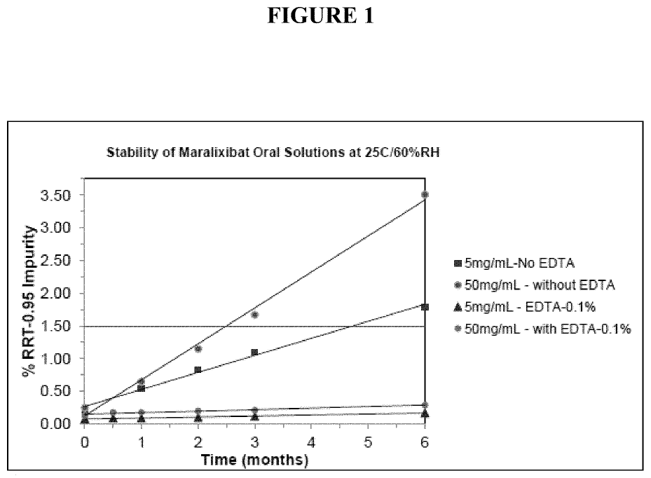

### DETAILED DESCRIPTION

Detailed embodiments of the present invention are disclosed herein; however, it is to be understood that the disclosed embodiments are merely illustrative of the invention that may be embodied in various forms. In addition, each of the examples given in connection with the various embodiments of the invention is intended to be illustrative, and not restrictive. Therefore, specific structural and functional details disclosed herein are not to be interpreted as limiting, but merely as a representative basis for teaching one skilled in the art to variously employ the present invention.

Unless defined otherwise, all technical and scientific terms used herein have the same meaning as commonly understood by one of ordinary skill in the art to which this invention belongs.

As used in this specification and the appended claims, the singular forms “a”, “an”, and “the” include plural references unless the context clearly dictates otherwise. Thus, for example, a reference to “a method” includes one or more methods, and/or steps of the type described herein and/or which will become apparent to those persons skilled in the art upon reading this disclosure.

The terms “treat” or “treatment” of a state, disorder or condition include: (1) preventing, delaying, or reducing the incidence and/or likelihood of the appearance of at least one clinical or sub-clinical symptom of the state, disorder or condition developing in a subject that may be afflicted with or predisposed to the state, disorder or condition but does not yet experience or display clinical or subclinical symptoms of the state, disorder or condition; or (2) inhibiting the state, disorder or condition, i.e., arresting, reducing or delaying the development of the disease or a relapse thereof or at least one clinical or sub-clinical symptom thereof, or (3) relieving the disease, i.e., causing regression of the state, disorder or condition or at least one of its clinical or sub-clinical symptoms. The benefit to a subject to be treated is either statistically significant or at least perceptible to the patient or to the physician.

A “subject” or “patient” or “individual” or “animal”, as used herein, refers to humans, veterinary animals (e.g., cats, dogs, cows, horses, sheep, pigs, etc.) and experimental animal models of diseases (e.g., mice, rats). In a preferred embodiment, the subject is a human.

As used herein the term “effective” applied to dose or amount refers to that quantity of a compound or pharmaceutical composition that is sufficient to result in a desired activity upon administration to a subject in need thereof. Note that when a combination of active ingredients is administered, the effective amount of the combination may or may not include amounts of each ingredient that would have been effective if administered individually. The exact amount required will vary from subject to subject, depending on the species, age, and general condition of the subject, the severity of the condition being treated, the particular drug or drugs employed, the mode of administration, and the like.

The phrase “pharmaceutically acceptable”, as used in connection with compositions of the invention, refers to molecular entities and other ingredients of such compositions that are physiologically tolerable and do not typically produce untoward reactions when administered to a mammal (e.g., a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans.

Ranges can be expressed herein as from “about” or “approximately” one particular value and/or to “about” or “approximately” another particular value. When such a range is expressed, another embodiment includes from the one particular value and/or to the other particular value. As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±20% of the recited value, e.g. “about 90%” may refer to the range of values from 71% to 99%.

By “comprising” or “containing” or “including” is meant that at least the named compound, element, particle, or method step is present in the composition or article or method, but does not exclude the presence of other compounds, materials, particles, or method steps, even if the other such compounds, material, particles, or method steps have the same function as what is named.

Compounds of the present invention include those described generally herein, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001, the entire contents of which are hereby incorporated by reference.

It is also to be understood that the mention of one or more method steps does not preclude the presence of additional method steps or intervening method steps between those steps expressly identified. Similarly, it is also to be understood that the mention of one or more components in a device or system does not preclude the presence of additional components or intervening components between those components expressly identified.

Unless otherwise stated, all crystalline forms of the compounds of the invention and salts thereof are also within the scope of the invention. The compounds of the invention may be isolated in various amorphous and crystalline forms, including without limitation forms which are anhydrous, hydrated, non-solvated, or solvated. Example hydrates include hemihydrates, monohydrates, dihydrates, and the like. In some embodiments, the compounds of the invention are anhydrous and non-solvated. By “anhydrous” is meant that the crystalline form of the compound contains essentially no bound water in the crystal lattice structure, i.e., the compound does not form a crystalline hydrate.

As used herein, “crystalline form” is meant to refer to a certain lattice configuration of a crystalline substance. Different crystalline forms of the same substance typically have different crystalline lattices (e.g., unit cells) which are attributed to different physical properties that are characteristic of each of the crystalline forms. In some instances, different lattice configurations have different water or solvent content. The different crystalline lattices can be identified by solid state characterization methods such as by X-ray powder diffraction (PXRD). Other characterization methods such as differential scanning calorimetry (DSC), thermogravimetric analysis (TGA), dynamic vapor sorption (DVS), solid state NMR, and the like further help identify the crystalline form as well as help determine stability and solvent/water content.

Crystalline forms of a substance include both solvated (e.g., hydrated) and non-solvated (e.g., anhydrous) forms. A hydrated form is a crystalline form that includes water in the crystalline lattice. Hydrated forms can be stoichiometric hydrates, where the water is present in the lattice in a certain water/molecule ratio such as for hemihydrates, monohydrates, dihydrates, etc. Hydrated forms can also be non-stoichiometric, where the water content is variable and dependent on external conditions such as humidity.

In some embodiments, the compounds of the invention are substantially isolated. By “substantially isolated” is meant that a particular compound is at least partially isolated from impurities. For example, in some embodiments a compound of the invention comprises less than about 50%, less than about 40%, less than about 30%, less than about 20%, less than about 15%, less than about 10%, less than about 5%, less than about 2.5%, less than about 1%, or less than about 0.5% of impurities. Impurities generally include anything that is not the substantially isolated compound including, for example, other crystalline forms and other substances.

The term “baseline” or “pre-administration baseline,” as used herein, refers to information gathered at the beginning of a study or an initial known value which is used for comparison with later data. A baseline is an initial measurement of a measurable condition that is taken at an early time point and used for comparison over time to look for changes in the measurable condition. For example, serum bile acid concentration in a patient before administration of a drug (baseline) and after administration of the drug. Baseline is an observation or value that represents the normal or beginning level of a measurable quality, used for comparison with values representing response to intervention or an environmental stimulus. The baseline is time “zero”, before participants in a study receive an experimental agent or intervention, or negative control. For example, “baseline” may refer in some instances 1) to the state of a measurable quantity just prior to the initiation of a clinical study or 2) the state of a measurable quantity just prior to altering a dosage level or composition administered to a patient from a first dosage level or composition to a second dosage level or composition.

The terms “level” and “concentration,” as used herein, are used interchangeably. For example, “high serum levels of bilirubin” may alternatively be phrased “high serum concentrations of bilirubin.”

The terms “normalized” or “normal range,” as used herein, indicates age-specific values that are within a range corresponding to a healthy individual (i.e., normal or normalized values). For example, the phrase “serum bilirubin concentrations were normalized within three weeks” means that serum bilirubin concentrations fell within a range known in the art to correspond to that of a healthy individual (i.e., within a normal and not e.g. an elevated range) within three weeks. In various embodiments, a normalized serum bilirubin concentration is from about 0.1 mg/dL to about 1.2 mg/dL. In various embodiments, a normalized serum bile acid concentration is from about 0 μmol/L to about 25 μmol/L.

The terms “ITCHRO(OBS)” and “ITCHRO” (alternatively, “ItchRO(Pt)”) as used herein, are used interchangeably with the qualification that the ITCHRO(OBS) scale is used to measure severity of pruritus in children under the age of 18 and the ITCHRO scale is used to measure severity of pruritus in adults of at least 18 years of age. Therefore, where ITCHRO(OBS) scale is mentioned with regard to an adult patient, the ITCHRO scale is the scale being indicated. Similarly, whenever the ITCHRO scale is mentioned with regard to a pediatric patient, the ITCHRO(OBS) scale is usually the scale being indicated (some older children were permitted to report their own scores as ITCHRO scores. The ITCHRO(OBS) scale ranges from 0 to 4 and the ITCHRO scale ranges from 0 to 10.

The term “bile acid” or “bile acids,” as used herein, includes steroid acids (and/or the carboxylate anion thereof), and salts thereof, found in the bile of an animal (e.g., a human), including, by way of non-limiting example, cholic acid, cholate, deoxycholic acid, deoxycholate, hyodeoxycholic acid, hyodeoxycholate, glycocholic acid, glycocholate, taurocholic acid, taurocholate, chenodeoxycholic acid, ursodeoxycholic acid (UDCA), ursodiol, a tauroursodeoxycholic acid, a glycoursodeoxycholic acid, a 7-B-methyl cholic acid, a methyl lithocholic acid, chenodeoxycholate, lithocholic acid, lithocolate, and the like. Taurocholic acid and/or taurocholate are referred to herein as TCA. Any reference to a bile acid used herein includes reference to a bile acid, one and only one bile acid, one or more bile acids, or to at least one bile acid. Therefore, the terms “bile acid,” “bile salt,” “bile acid/salt,” “bile acids,” “bile salts,” and “bile acids/salts” are, unless otherwise indicated, utilized interchangeably herein. Any reference to a bile acid used herein includes reference to a bile acid or a salt thereof. Furthermore, pharmaceutically acceptable bile acid esters are optionally utilized as the “bile acids” described herein, e.g., bile acids/salts conjugated to an amino acid (e.g., glycine or taurine). Other bile acid esters include, e.g., substituted or unsubstituted alkyl ester, substituted or unsubstituted heteroalkyl esters, substituted or unsubstituted aryl esters, substituted or unsubstituted heteroaryl esters, or the like. For example, the term “bile acid” includes cholic acid conjugated with either glycine or taurine:glycocholate and taurocholate, respectively (and salts thereof). Any reference to a bile acid used herein includes reference to an identical compound naturally or synthetically prepared. Furthermore, it is to be understood that any singular reference to a component (bile acid or otherwise) used herein includes reference to one and only one, one or more, or at least one of such components. Similarly, any plural reference to a component used herein includes reference to one and only one, one or more, or at least one of such components, unless otherwise noted.

The term “composition,” as used herein includes the disclosure of both a composition and a composition administered in a method as described herein. Furthermore, in some embodiments, the composition of the present invention is or comprises a “formulation,” an oral dosage form or a rectal dosage form as described herein.

The terms “effective amount” or “therapeutically effective amount” as used herein, refer to a sufficient amount of at least one agent (e.g., a therapeutically active agent) being administered which achieve a desired result in a subject or individual, e.g., to relieve to some extent one or more symptoms of a disease or condition being treated. In certain instances, the result is a reduction and/or alleviation of the signs, symptoms, or causes of a disease, or any other desired alteration of a biological system. In certain instances, an “effective amount” for therapeutic uses is the amount of the composition comprising an agent as set forth herein required to provide a clinically significant decrease in a disease. An appropriate “effective” amount in any individual case is determined using any suitable technique, such as a dose escalation study. In some embodiments, a “therapeutically effective amount,” or an “effective amount” of an ASBTI refers to a sufficient amount of an ASBTI to treat cholestasis or a cholestatic liver disease in a subject or individual.

The terms “administer,” “administering”, “administration,” and the like, as used herein, refer to the methods that may be used to enable delivery of agents or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. Administration techniques that are optionally employed with the agents and methods described herein are found in sources e.g., Goodman and Gilman, The Pharmacological Basis of Therapeutics, current ed.; Pergamon; and Remington's, Pharmaceutical Sciences (current edition), Mack Publishing Co., Easton, Pa., all of which are incorporated herein by reference in their entirety for all purposes. In certain embodiments, the agents and compositions described herein are administered orally.

The term “ASBT inhibitor” refers to a compound that inhibits apical sodium-dependent bile transport or any recuperative bile salt transport. The term Apical Sodium-dependent Bile Transporter (ASBT) is used interchangeably with the term Ileal Bile Acid Transporter (IBAT).

Bile Acid

Bile contains water, electrolytes and a numerous organic molecules including bile acids, cholesterol, phospholipids and bilirubin. Bile is secreted from the liver and stored in the gall bladder, and upon gall bladder contraction, due to ingestion of a fatty meal, bile passes through the bile duct into the intestine. Bile acids/salts are critical for digestion and absorption of fats and fat-soluble vitamins in the small intestine. Adult humans produce 400 to 800 mL of bile daily. The secretion of bile can be considered to occur in two stages. Initially, hepatocytes secrete bile into canaliculi, from which it flows into bile ducts and this hepatic bile contains large quantities of bile acids, cholesterol and other organic molecules. Then, as bile flows through the bile ducts, it is modified by addition of a watery, bicarbonate-rich secretion from ductal epithelial cells. Bile is concentrated, typically five-fold, during storage in the gall bladder.

The flow of bile is lowest during fasting, and a majority of that is diverted into the gallbladder for concentration. When chyme from an ingested meal enters the small intestine, acid and partially digested fats and proteins stimulate secretion of cholecystokinin and secretin, both of which are important for secretion and flow of bile. Cholecystokinin (cholecysto=gallbladder and kinin=movement) is a hormone which stimulates contractions of the gallbladder and common bile duct, resulting in delivery of bile into the gut. The most potent stimulus for release of cholecystokinin is the presence of fat in the duodenum. Secretin is a hormone secreted in response to acid in the duodenum, and it simulates biliary duct cells to secrete bicarbonate and water, which expands the volume of bile and increases its flow out into the intestine.

Bile acids/salts are derivatives of cholesterol. Cholesterol, ingested as part of the diet or derived from hepatic synthesis, are converted into bile acids/salts in the hepatocyte. Examples of such bile acids/salts include cholic and chenodeoxycholic acids, which are then conjugated to an amino acid (such as glycine or taurine) to yield the conjugated form that is actively secreted into cannaliculi. The most abundant of the bile salts in humans are cholate and deoxycholate, and they are normally conjugated with either glycine or taurine to give glycocholate or taurocholate respectively.

Free cholesterol is virtually insoluble in aqueous solutions, however in bile it is made soluble by the presence of bile acids/salts and lipids. Hepatic synthesis of bile acids/salts accounts for the majority of cholesterol breakdown in the body. In humans, roughly 500 mg of cholesterol are converted to bile acids/salts and eliminated in bile every day. Therefore, secretion into bile is a major route for elimination of cholesterol. Large amounts of bile acids/salts are secreted into the intestine every day, but only relatively small quantities are lost from the body. This is because approximately 95% of the bile acids/salts delivered to the duodenum are absorbed back into blood within the ileum, by a process is known as “Enterohepatic Recirculation”.

Venous blood from the ileum goes straight into the portal vein, and hence through the sinusoids of the liver. Hepatocytes extract bile acids/salts very efficiently from sinusoidal blood, and little escapes the healthy liver into systemic circulation. Bile acids/salts are then transported across the hepatocytes to be resecreted into canaliculi. The net effect of this enterohepatic recirculation is that each bile salt molecule is reused about 20 times, often two or three times during a single digestive phase. Bile biosynthesis represents the major metabolic fate of cholesterol, accounting for more than half of the approximate 800 mg/day of cholesterol that an average adult uses up in metabolic processes. In comparison, steroid hormone biosynthesis consumes only about 50 mg of cholesterol per day. Much more that 400 mg of bile salts is required and secreted into the intestine per day, and this is achieved by re-cycling the bile salts. Most of the bile salts secreted into the upper region of the small intestine are absorbed along with the dietary lipids that they emulsified at the lower end of the small intestine. They are separated from the dietary lipid and returned to the liver for re-use. Recycling thus enables 20-30 g of bile salts to be secreted into the small intestine each day.

Bile acids/salts are amphipathic, with the cholesterol-derived portion containing both hydrophobic (lipid soluble) and polar (hydrophilic) moieties while the amino acid conjugate is generally polar and hydrophilic. This amphipathic nature enables bile acids/salts to carry out two important functions: emulsification of lipid aggregates and solubilization and transport of lipids in an aqueous environment. Bile acids/salts have detergent action on particles of dietary fat which causes fat globules to break down or to be emulsified. Emulsification is important since it greatly increases the surface area of fat available for digestion by lipases which cannot access the inside of lipid droplets. Furthermore, bile acids/salts are lipid carriers and are able to solubilize many lipids by forming micelles and are critical for transport and absorption of the fat-soluble vitamins.

The term “non-systemic” or “minimally absorbed,” as used herein, refers to low systemic bioavailability and/or absorption of an administered compound. In some embodiments, a non-systemic compound is a compound that is substantially not absorbed systemically. In some embodiments, ASBTI compositions described herein deliver the ASBTI to the distal ileum, colon, and/or rectum and not systemically (e.g., a substantial portion of the ASBTI is not systemically absorbed. In some embodiments, the systemic absorption of a non-systemic compound is <0.1%, <0.3%, <0.5%, <0.6%, <0.7%, <0.8%, <0.9%, <1%, <1.5%, <2%, <3%, or <5% of the administered dose (wt. % or mol %). In some embodiments, the systemic absorption of a non-systemic compound is <10% of the administered dose. In some embodiments, the systemic absorption of a non-systemic compound is <15% of the administered dose. In some embodiments, the systemic absorption of a non-systemic compound is <25% of the administered dose. In an alternative approach, a non-systemic ASBTI is a compound that has lower systemic bioavailability relative to the systemic bioavailability of a systemic ASBTI (e.g., compound 100A, 100C). In some embodiments, the bioavailability of a non-systemic ASBTI described herein is <30%, <40%, <50%, <60%, or <70% of the bioavailability of a systemic ASBTI (e.g., compound 100A, 100C).

In another alternative approach, compositions described herein are formulated to deliver <10% of the administered dose of the ASBTI systemically. In some embodiments, the compositions described herein are formulated to deliver <20% of the administered dose of the ASBTI systemically. In some embodiments, the compositions described herein are formulated to deliver <30% of the administered dose of the ASBTI systemically. In some embodiments, the compositions described herein are formulated to deliver <40% of the administered dose of the ASBTI systemically. In some embodiments, the compositions described herein are formulated to deliver <50% of the administered dose of the ASBTI systemically. In some embodiments, the compositions described herein are formulated to deliver <60% of the administered dose of the ASBTI systemically. In some embodiments, the compositions described herein are formulated to deliver <70% of the administered dose of the ASBTI systemically. In some embodiments, systemic absorption is determined in any suitable manner, including the total circulating amount, the amount cleared after administration, or the like.

ASBTIs

In one aspect, the present compositions comprise an ASBTI as an active agent. Various ASBTIs are suitable for use with the compositions of the present disclosure.

In some embodiments, the ASBTI is

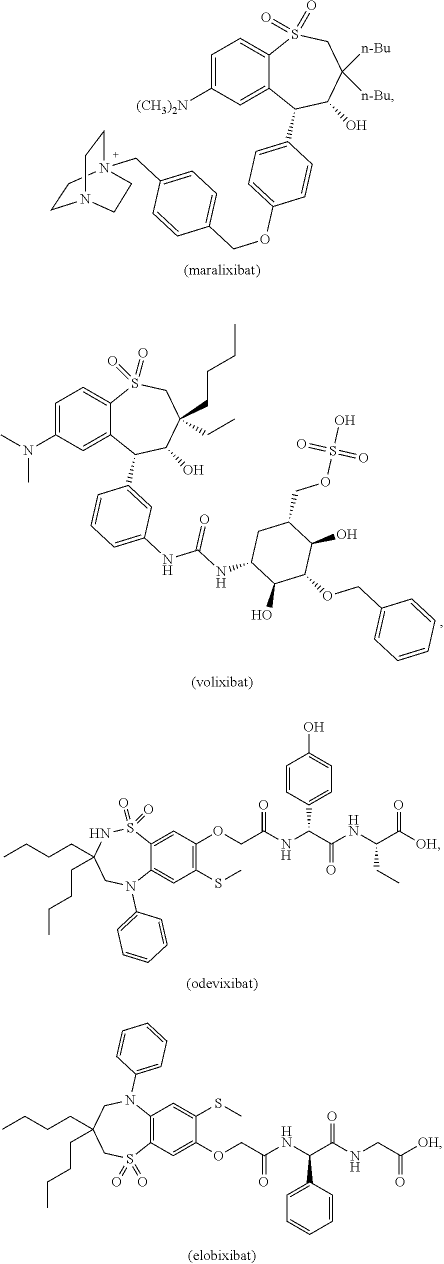

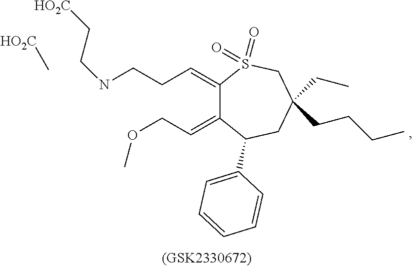

or a pharmaceutically acceptable salt thereof. In some embodiments, the ASBTI is maralixibat, or a pharmaceutically acceptable salt thereof. In some embodiments, the ASBTI is maralixibat chloride, or a pharmaceutically acceptable alternative salt thereof. In various embodiments, the ASBTI is volixibat, or a pharmaceutically acceptable salt thereof. In various embodiments, the ASBTI is odevixibat, or a pharmaceutically acceptable salt thereof. In some embodiments, the ASBTI is elobixibat, or a pharmaceutically acceptable salt thereof. In various embodiments, the ASBTI is GSK2330672, or a pharmaceutically acceptable salt thereof.

In various embodiments, the ASBTI may be a free base or a pharmaceutically acceptable salt of the compounds disclosed herein.

In some embodiments, the ASBTI is

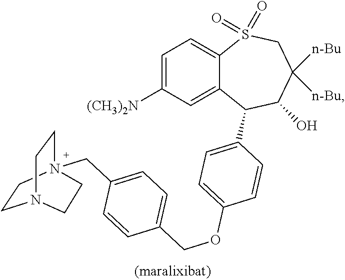

or a pharmaceutically acceptable salt thereof.

In some embodiments, the ASBTI is

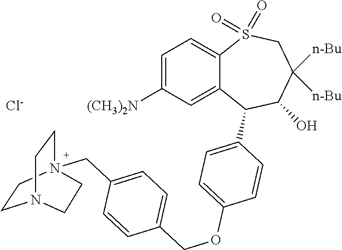

(maralixibat chloride, LUM-001, SHP625, lopixibat chloride), or an alternative pharmaceutically acceptable salt thereof.

In some embodiments, the ASBTI is

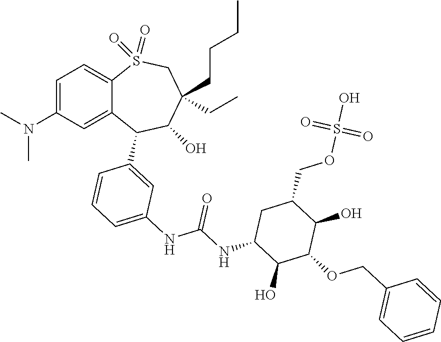 [SMILES: CCCC[C@]1(CC)CS(=O)(=O)c2ccc(N(C)C)cc2[C@@H](c2cccc(NC(=O)N[C@@H]3C[C@H](COS(=O)(=O)O)[C@@H](O)[C@H](OCc4ccccc4)[C@H]3O)c2)[C@H]1O]

(volixibat, (2R,3R,4S,5R,6R)-4-benzyloxy-6-{3-[3-((3S,4R,5R)-3-butyl-7-dimethylamino-3-ethyl-4-hydroxy-1,1-dioxo-2,3,4,5-tetrahydro-1H-benzo[b]thiepin-5-yl)-phenyl]-ureido}-3,5-dihydroxy-tetrahydro-pyran-2-ylmethyl) hydrogen sulfate), or a pharmaceutically acceptable salt thereof.

In some embodiments, the ASBTI is

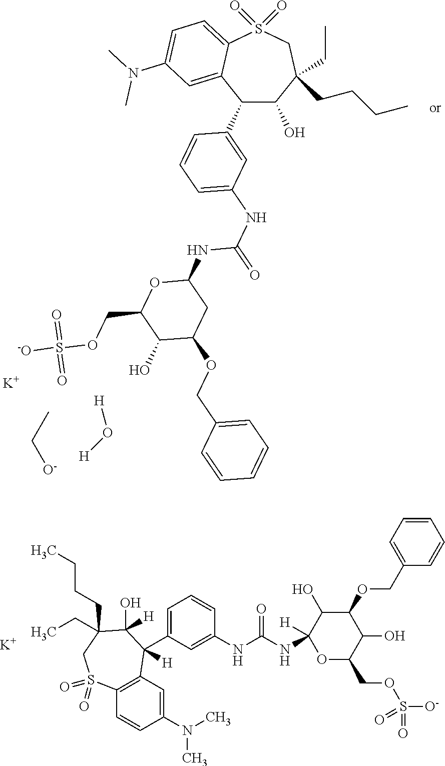

(LUM-002; SHP626; SAR548304; volixibat potassium) or an alternative pharmaceutically acceptable salt thereof.

In various embodiments the ASBTI is

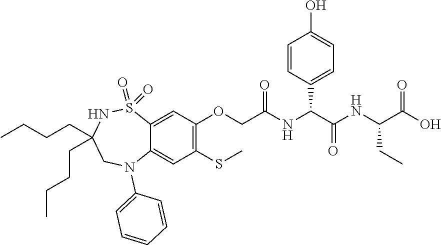 [SMILES: CCCCC1(CCCC)CN(c2ccccc2)c2cc(SC)c(OCC(=O)N[C@@H](C(=O)N[C@@H](CC)C(=O)O)c3ccc(O)cc3)cc2S(=O)(=O)N1]

(odevixibat; AZD8294; WHO10706; AR-H064974; SCHEMBL946468; A4250; 1,1-dioxo-3,3-dibutyl-5-phenyl-7-methylthio-8-(N—{(R)-a-[N—((S)-1-carboxypropyl) carbamoyl]-4-hydroxybenzyl}carbamoylmethoxy)-2,3,4,5-tetrahydro-1,2,5-benzothiadiazepine), or a pharmaceutically acceptable salt thereof.

In some embodiments, the ASBTI is

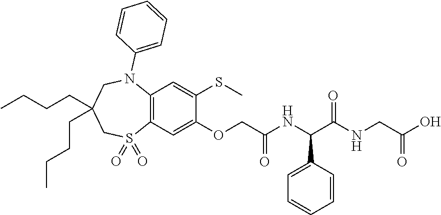 [SMILES: CCCCC1(CCCC)CN(c2ccccc2)c2cc(SC)c(OCC(=O)N[C@@H](C(=O)NCC(=O)O)c3ccccc3)cc2S(=O)(=O)C1]

(elobixibat; 2-[[(2R)-2-[[2-[(3,3-dibutyl-7-methylsulfanyl-1,1-dioxo-5-phenyl-2,4-dihydro-1λ6,5-benzothiazepin-8-yl)oxy]acetyl]amino]-2-phenylacetyl]amino]acetic acid), or a pharmaceutically acceptable salt thereof.

In some embodiments, the ASBTI is

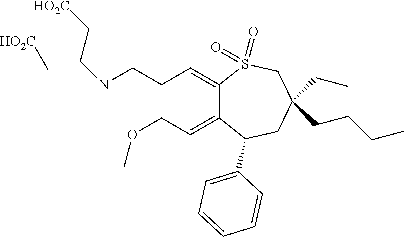

(GSK2330672; linerixibat; 3-((((3R,5R)-3-butyl-3-ethyl-7-(methyloxy)-1,1-dioxido-5-phenyl-2,3,4,5-tetrahydro-1,4-benzothiazepin-8-yl)methyl)amino)pentanedioic acid), or a pharmaceutically acceptable salt thereof.

In some embodiments, ASBTIs described herein are synthesized as described in, for example, WO 96/05188, U.S. Pat. Nos. 5,994,391; 7,238,684; 6,906,058; 6,020,330; and 6,114,322.

In some embodiments, the ASBTI used in the methods or compositions of the present invention is maralixibat (SHP625), volixibat (SHP626), or odevixibat (A4250), or a pharmaceutically acceptable salt thereof.

In some embodiments, the ASBTI used in the methods or compositions of the present invention is maralixibat, or a pharmaceutically acceptable salt thereof. In some embodiments, the ASBTI used in the methods or compositions of the present invention is maralixibat chloride.

In some embodiments, the ASBTI used in the methods or compositions of the present invention is volixibat, or a pharmaceutically acceptable salt thereof.

In some embodiments, the ASBTI used in the methods or compositions of the present invention is odevixibat, or a pharmaceutically acceptable salt thereof.

In some embodiments, the ASBTI used in the methods or compositions of the present invention is elobixibat, or a pharmaceutically acceptable salt thereof.

In some embodiments, the ASBTI used in the methods or compositions of the present invention is GSK2330672, or a pharmaceutically acceptable salt thereof.

In some embodiments, the ASBTI may comprise a mixture of different ASBTIs; for example, the ASBTI may be a composition comprising maralixibat (e.g., maralixibat chloride), volixibat, odevixibat, GSK2330672, elobixibat, or various combinations thereof.

Pediatric Dosage Formulations and Compositions

Provided herein, in certain embodiments, is a pediatric dosage formulation or composition comprising a therapeutically effective amount of any compound described herein. In certain instances, the pharmaceutical composition comprises an ASBT inhibitor (e.g., any ASBTI described herein), a preservative, and an antioxidant.

Preservative

In certain embodiments, the compositions of the present invention comprise a preservative. In certain embodiments, the preservative is an antimicrobial preservative.

In certain embodiments, the antimicrobial preservative is selected from the group consisting of propylene glycol, ethyl alcohol, glycerin, benzalkonium chloride, benzethonium chloride, benzoic acid, benzyl alcohol, butylparaben, cetrimide (cetyltrimethylammonium bromide), cetrimonium bromide, cetylpyridinium chloride, chlorhexidine, chlorobutanol, chlorocresol, cresol, ethylparaben, methylparaben, phenol, phenoxyethanol, phenylethyl alcohol, phenylmercuric acetate, phenylmercuric borate, phenylmercuric nitrate, propylparaben, sodium benzoate, sodium dehydroacetate, sodium propionate, sorbic acid, potassium sorbate, thimerosal, thymol, and combinations thereof.

In certain embodiments, the preservative is propylene glycol.

In certain embodiments, the preservative is present in an amount of at least about 10% w/w of the composition. In certain embodiments, the preservative is present in an amount of at least about 20% w/w of the composition. In certain embodiments, the preservative is present in an amount of at least about 25% w/w of the composition. In certain embodiments, the preservative is present in an amount of at least about 30% w/w of the composition.

In certain embodiments, the preservative is present in an amount of from about 30% to about 40% of the composition.

In certain embodiments, the preservative is present in an amount of from about 32% to about 37% of the composition. In certain embodiments, the preservative is present in an amount of from about 33% to about 36% of the composition.

In certain embodiments, the preservative is present in an amount of about 33% of the composition. In certain embodiments, the preservative is present in an amount of about 34% of the composition. In certain embodiments, the preservative is present in an amount of about 35% of the composition.

Antioxidant

In certain embodiments, the compositions of the present invention comprise an antioxidant. In certain embodiments, the antioxidant is selected from the group consisting of an aminocarboxylic acid, an aminopolycarboxylic acid, ascorbic acid, ascorbyl palmitate, butylated hydroxyanisole, butylated hydroxytoluene, monothioglycerol, sodium ascorbate, sodium formaldehyde sulfoxylate, sodium metabisulfite, BHT, BHA, sodium bisulfite, vitamin E or a derivative thereof, propyl gallate, and combinations thereof.

In certain embodiments, the antioxidant is an aminopolycarboxylic acid selected from EDTA (ethylenediaminetetraacetic acid), DTPA (diethylenetriaminepentaacetic acid), EGTA (ethylene glycol-bis(β-aminoethyl ether)-N,N,N′,N′-tetraacetic acid), NTA (nitrilotriacetic acid), BAPTA (1,2-bis(o-aminophenoxy)ethane-N,N,N′,N′-tetraacetic acid), NOTA (2,2′,2″-(1,4,7-triazonane-1,4,7-triyl)triacetic acid), DOTA (tetracarboxylic acid), and EDDHA (ethylenediamine-N,N′-bis(2-hydroxyphenylacetic acid)

In certain embodiments, the antioxidant is EDTA.

In certain embodiments, the antioxidant is present in an amount of about 0.001% to about 1% w/w of the composition. In certain embodiments, the antioxidant is present in an amount of about 0.005% to about 0.75% w/w of the composition. In certain embodiments, the antioxidant is present in an amount of about 0.01% to about 0.5% w/w of the composition. In certain embodiments, the antioxidant is present in an amount of about 0.05% to about 0.25% w/w of the composition. In certain embodiments, the antioxidant is present in an amount of about 0.075% to about 0.2% w/w of the composition. In certain embodiments, the antioxidant is present in an amount of about 0.1% w/w of the composition.

In certain embodiments, suitable dosage forms for the pediatric dosage formulation or composition include liquid dosage forms. By way of non-limiting example, liquid dosage forms may include aqueous or non-aqueous oral dispersions, liquids, gels, syrups, elixirs, slurries, suspensions, and solutions, controlled release formulations, sustained release formulations and fast acting formulations. In some embodiments, provided herein is a pharmaceutical composition wherein the pediatric dosage form is selected from a solution, syrup, suspension, and elixir.

In another aspect, provide herein is a pharmaceutical composition wherein at least one excipient is a flavoring agent or a sweetener. In some embodiments, provided herein is a coating. In some embodiments, provided herein is a taste-masking technology selected from coating of drug particles with a taste-neutral polymer by spray-drying, wet granulation, fluidized bed, and microencapsulation; coating with molten waxes of a mixture of molten waxes and other pharmaceutical adjuvants; entrapment of drug particles by complexation, flocculation or coagulation of an aqueous polymeric dispersion; adsorption of drug particles on resin and inorganic supports; and solid dispersion wherein a drug and one or more taste neutral compounds are melted and cooled, or co-precipitated by a solvent evaporation. In some embodiments, provided herein is a delayed or sustained release formulation comprising drug particles or granules in a rate controlling polymer or matrix.

Suitable sweeteners include sucrose, glucose, fructose or intense sweeteners, i.e. agents with a high sweetening power when compared to sucrose (e.g. at least 10 times sweeter than sucrose). Suitable intense sweeteners comprise aspartame, saccharin, sodium or potassium or calcium saccharin, acesulfame potassium, sucralose, alitame, xylitol, cyclamate, neomate, neohesperidine dihydrochalcone or mixtures thereof, thaumatin, palatinit, stevioside, rebaudioside, Magnasweet®. The total concentration of the sweeteners may range from effectively zero to about 300 mg/ml based on the liquid composition.

In order to increase the palatability of the liquid composition upon reconstitution with an aqueous medium, one or more taste-making agents may be added to the composition in order to mask the taste of the ASBT inhibitor. A taste-masking agent can be a sweetener, a flavoring agent or a combination thereof. The taste-masking agents typically provide up to about 0.1% or 5% by weight of the total pharmaceutical composition. In a preferred embodiment of the present invention, the composition contains both sweetener(s) and flavor(s).

A flavoring agent herein is a substance capable of enhancing taste or aroma of a composition. Suitable natural or synthetic flavoring agents can be selected from standard reference books, for example Fenaroli's Handbook of Flavor Ingredients, 3rd edition (1995). Non-limiting examples of flavoring agents and/or sweeteners useful in the formulations described herein, include, e.g., acacia syrup, acesulfame K, alitame, anise, apple, aspartame, banana, Bavarian cream, berry, black currant, butterscotch, calcium citrate, camphor, caramel, cherry, cherry cream, chocolate, cinnamon, bubble gum, citrus, citrus punch, citrus cream, cotton candy, cocoa, cola, cool cherry, cool citrus, cyclamate, cylamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, monoammonium glyrrhizinate (MagnaSweet®), maltol, mannitol, maple, marshmallow, menthol, mint cream, mixed berry, neohesperidine DC, neotame, orange, pear, peach, peppermint, peppermint cream, Prosweet® Powder, raspberry, root beer, rum, saccharin, safrole, sorbitol, spearmint, spearmint cream, strawberry, strawberry cream, stevia, sucralose, sucrose, sodium saccharin, saccharin, aspartame, acesulfame potassium, mannitol, talin, sylitol, sucralose, sorbitol, Swiss cream, tagatose, tangerine, thaumatin, tutti frutti, vanilla, walnut, watermelon, wild cherry, wintergreen, xylitol, or any combination of these flavoring ingredients, e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof. Flavoring agents can be used singly or in combinations of two or more. In some embodiments, the composition comprises a sweetening agent or flavoring agent in a concentration ranging from about 0.001% to about 5.0% the volume of the composition. In one embodiment, the composition comprises a sweetening agent or flavoring agent in a concentration ranging from about 0.001% to about 1.0% the volume of the aqueous dispersion. In another embodiment, the composition comprises a sweetening agent or flavoring agent in a concentration ranging from about 0.002% to about 0.5% the volume of the composition. In yet another embodiment, the composition comprises a sweetening agent or flavoring agent in a concentration ranging from about 0.003% to about 0.25% the volume of the composition. In yet another embodiment, the composition comprises a sweetening agent or flavoring agent in a concentration ranging from about 0.005% to about 0.1% the volume of the composition.

In certain embodiments, a pediatric pharmaceutical composition described herein includes one or more compound described herein as an active ingredient in free-acid or free-base form, or in a pharmaceutically acceptable salt form. In some embodiments, the compounds described herein are utilized as an N-oxide or in a crystalline or amorphous form (i.e., a polymorph). In some situations, a compound described herein exists as tautomers. All tautomers are included within the scope of the compounds presented herein. In certain embodiments, a compound described herein exists in an unsolvated or solvated form, wherein solvated forms comprise any pharmaceutically acceptable solvent, e.g., water, ethanol, and the like. The solvated forms of the compounds presented herein are also considered to be described herein.

A “carrier” for pediatric pharmaceutical compositions includes, in some embodiments, a pharmaceutically acceptable excipient and is selected on the basis of compatibility with compounds described herein, such as ASBTIs, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. See, e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), all of which references are incorporated herein by reference in their entirety for all purposes.

Moreover, in certain embodiments, the pediatric pharmaceutical compositions described herein are formulated as a dosage form. As such, in some embodiments, provided herein is a dosage form comprising a compound described herein, suitable for administration to an individual. In certain embodiments, suitable dosage forms include, by way of non-limiting example, aqueous oral dispersions, liquids, gels, syrups, elixirs, slurries, suspensions, liquid oral dosage forms, controlled release formulations, fast acting formulations, delayed release formulations, extended release formulations, sustained release formulations, pulsatile release formulations, and mixed immediate release and controlled release formulations.

In some embodiments, ASBTIs, or other compounds described herein are orally administered in association with a carrier suitable for delivery to the distal gastrointestinal tract (e.g., distal ileum, colon, and/or rectum).

In certain embodiments, a pediatric composition described herein comprises an ASBTI, or other compounds described herein in association with a matrix (e.g., a matrix comprising hypermellose) that allows for controlled release of an active agent in the distal part of the ileum and/or the colon. In some embodiments, a composition comprises a polymer that is pH sensitive (e.g., a MMX™ matrix from Cosmo Pharmaceuticals) and allows for controlled release of an active agent in the distal part of the ileum. Examples of such pH sensitive polymers suitable for controlled release include and are not limited to polyacrylic polymers (e.g., anionic polymers of methacrylic acid and/or methacrylic acid esters, e.g., Carbopol® polymers) that comprise acidic groups (e.g., —COOH, —SO3H) and swell in basic pH of the intestine (e.g., pH of about 7 to about 8). In some embodiments, a composition suitable for controlled release in the distal ileum comprises microparticulate active agent (e.g., micronized active agent). In some embodiments, a non-enzymatically degrading poly(dl-lactide-co-glycolide) (PLGA) core is suitable for delivery of an enteroendocrine peptide secretion enhancing agent to the distal ileum. In some embodiments, a dosage form comprising an enteroendocrine peptide secretion enhancing agent is coated with an enteric polymer (e.g., Eudragit® S-100, cellulose acetate phthalate, polyvinylacetate phthalate, hydroxypropylmethylcellulose phthalate, anionic polymers of methacrylic acid, methacrylic acid esters or the like) for site specific delivery to the distal ileum and/or the colon. In some embodiments, bacterially activated systems are suitable for targeted delivery to the distal part of the ileum. Examples of micro-flora activated systems include dosage forms comprising pectin, galactomannan, and/or Azo hydrogels and/or glycoside conjugates (e.g., conjugates of D-galactoside, β-D-xylopyranoside or the like) of the active agent. Examples of gastrointestinal micro-flora enzymes include bacterial glycosidases such as, for example, D-galactosidase, β-D-glucosidase, α-L-arabinofuranosidase, β-D-xylopyranosidase or the like.

The pediatric pharmaceutical composition described herein optionally include an additional therapeutic compound described herein and one or more pharmaceutically acceptable additives such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof.

Liquid Dosage Forms

The pharmaceutical liquid dosage forms of the invention may be prepared according to techniques well-known in the art of pharmacy.

A solution refers to a liquid pharmaceutical formulation wherein the active ingredient is dissolved in the liquid. Pharmaceutical solutions of the invention include syrups and elixirs. A suspension refers to a liquid pharmaceutical formulation wherein the active ingredient is in a precipitate in the liquid.

In a liquid dosage form, it is desirable to have a particular pH and/or to be maintained within a specific pH range. In order to control the pH, a suitable buffer system can be used. In addition, the buffer system should have sufficient capacity to maintain the desired pH range. Examples of the buffer system useful in the present invention include but are not limited to, citrate buffers, phosphate buffers, or any other suitable buffer known in the art. Preferably the buffer system include sodium citrate, potassium citrate, sodium bicarbonate, potassium bicarbonate, sodium dihydrogen phosphate and potassium dihydrogen phosphate, etc. The concentration of the buffer system in the final suspension varies according to factors such as the strength of the buffer system and the pH/pH ranges required for the liquid dosage form. In one embodiment, the concentration is within the range of 0.005 to 0.5 w/v % in the final liquid dosage form.

The pharmaceutical composition comprising the liquid dosage form of the present invention can also include a suspending/stabilizing agent to prevent settling of the active material. Over time the settling could lead to caking of the active to the inside walls of the product pack, leading to difficulties with redispersion and accurate dispensing. Suitable stabilizing agents include but are not limited to, the polysaccharide stabilizers such as xanthan, guar and tragacanth gums as well as the cellulose derivatives HPMC (hydroxypropyl methylcellulose), methyl cellulose and Avicel RC-591 (microcrystalline cellulose/sodium carboxymethyl cellulose). In another embodiment, polyvinylpyrrolidone (PVP) can also be used as a stabilizing agent.

In addition to the aforementioned components, the ASBTI oral composition can also optionally contain other excipients commonly found in pharmaceutical compositions such as alternative solvents, taste-masking agents, antioxidants, fillers, acidifiers, enzyme inhibitors and other components as described in Handbook of Pharmaceutical Excipients, Rowe et al., Eds., 4th Edition, Pharmaceutical Press (2003), which is hereby incorporated by reference in its entirety for all purposes.

Addition of an alternative solvent may help increase solubility of an active ingredient in the liquid dosage form, and consequently the absorption and bioavailability inside the body of a subject. Preferably the alternative solvents include methanol, ethanol or propylene glycol and the like.

In another aspect, the present invention provides a process for preparing the liquid dosage form. The process comprises steps of bringing ASBTI or its pharmaceutically acceptable salts thereof into mixture with the components including glycerol or syrup or the mixture thereof, a preservative, a buffer system and a suspending/stabilizing agent, etc., in a liquid medium. In general, the liquid dosage form is prepared by uniformly and intimately mixing these various components in the liquid medium. For example, the components such as glycerol or syrup or the mixture thereof, a preservative, a buffer system and a suspending/stabilizing agent, etc., can be dissolved in water to form the aqueous solution, then the active ingredient can be then dispersed in the aqueous solution to form a suspension.

In some embodiments, the liquid dosage form provided herein can be in a volume of between about 0.001 ml to about 50 ml. In some embodiments, the liquid dosage form provided herein can be in a volume of between about 0.01 ml to about 20 ml. In some embodiments, the liquid dosage form provided herein can be in a volume of between about 0.05 ml to about 10 ml. In some embodiments, the liquid dosage form provided herein can be in a volume of between about 0.1 ml to about 5 ml. In some embodiments, the liquid dosage form provided herein can be in a volume of between about 0.1 ml to about 3 ml.

In some embodiments, the liquid dosage form provided herein can be in a volume of about 0.1 ml, or about 0.15 ml, or about 0.2 ml, or about 0.25 ml, or about 0.3 ml, or about 0.35 ml, or about 0.4 ml, or about 0.45 ml, or about 0.5 ml, or about 0.55 ml, or about 0.6 ml, or about 0.65 ml, or about 0.7 ml, or about 0.75 ml, or about 0.8 ml, or about 0.85 ml, or about 0.9 ml, or about 0.95 ml, or about 1.00 ml, or about 1.05 ml, or about 1.1 ml, or about 1.2 ml, or about 1.25 ml, or about 1.5 ml, or about 1.75 ml, or about 2.00 ml, or about 2.25 ml, or about 2.5 ml, or about 2.75 ml, or about 3.00 ml.

In some embodiments, the ASBTI can be in an amount ranging from about 0.001% to about 90% of the total volume. In some embodiments, the ASBTI can be in an amount ranging from about 0.01% to about 80% of the total volume. In some embodiments, the ASBTI can be in an amount ranging from about 0.1% to about 50% of the total volume. In some embodiments, the ASBTI can be in an amount ranging from about 0.2% to about 25% of the total volume. In some embodiments, the ASBTI can be in an amount ranging from about 0.5% to about 10% of the total volume. In some embodiments, the ASBTI can be in an amount ranging from about 0.5% to about 5% of the total volume.

In one embodiment, the compositions described herein can be in a liquid volume of about 0.01 ml to about 50 ml, or from about 0.1 ml to about 5 ml, and the active ingredient (e.g., maralixibat) can be in an amount ranging from about 0.001 mg/ml to about 500 mg/ml, or from about 0.5 mg/ml to about 100 mg/ml, or from about 1 mg/ml to about 80 mg/ml, or from about 5 mg/ml to about 50 mg/ml, or about 5 mg/ml, or about 9.5 mg/ml, or about 10 mg/ml, or about 15 mg/ml, or about 20 mg/ml, or about 25 mg/ml, or about 30 mg/ml, or about 35 mg/ml, or about 40 mg/ml or about 50 mg/ml.

In one non-limiting embodiment, the concentration of maralixibat in the composition is 10 mg/ml based on maralixibat chloride.

In one non-limiting embodiment, the concentration of maralixibat in the composition is 9.5 mg/ml based on maralixibat free base.

In certain embodiments, the compositions described herein are stable for at least 1 month at room temperature. In certain embodiments, the compositions described herein are for at least 2 months at room temperature. In certain embodiments, the compositions described herein are for at least 3 months at room temperature. In certain embodiments, the compositions described herein are for at least 6 months at room temperature. In one embodiment, the composition is stable for at least 1 year at room temperature. In one embodiment, the composition is stable for at least 18 months at room temperature. In one embodiment, the composition is stable for at least 2 years at room temperature.

In certain embodiments, the compositions described herein are liquid compositions for oral administration.

Routes of Administration, Dosage Forms, and Dosing Regimens

In some embodiments, the compositions described herein, and the compositions administered in the methods described herein are formulated to inhibit bile acid reuptake or reduce serum or hepatic bile acid levels. In certain embodiments, the compositions described herein are formulated for oral administration. In some embodiments, such formulations are administered orally. In some embodiments, for oral administration the compositions described herein are formulated for oral administration and enteric delivery to the colon.

In certain embodiments, the compositions or methods described herein are non-systemic. In some embodiments, compositions described herein deliver the ASBTI to the distal ileum, colon, and/or rectum and not systemically (e.g., a substantial portion of the enteroendocrine peptide secretion enhancing agent is not systemically absorbed). In some embodiments, oral compositions described herein deliver the ASBTI to the distal ileum, colon, and/or rectum and not systemically (e.g., a substantial portion of the enteroendocrine peptide secretion enhancing agent is not systemically absorbed).

In certain embodiments, non-systemic compositions described herein deliver less than 90% w/w of the ASBTI systemically. In certain embodiments, non-systemic compositions described herein deliver less than 80% w/w of the ASBTI systemically. In certain embodiments, non-systemic compositions described herein deliver less than 70% w/w of the ASBTI systemically. In certain embodiments, non-systemic compositions described herein deliver less than 60% w/w of the ASBT1 systemically. In certain embodiments, non-systemic compositions described herein deliver less than 50% w/w of the ASBTI systemically. In certain embodiments, non-systemic compositions described herein deliver less than 40% w/w of the ASBTI systemically. In certain embodiments, non-systemic compositions described herein deliver less than 30% w/w of the ASBTI systemically. In certain embodiments, non-systemic compositions described herein deliver less than 25% w/w of the ASBTI systemically. In certain embodiments, non-systemic compositions described herein deliver less than 20% w/w of the ASBTI systemically. In certain embodiments, non-systemic compositions described herein deliver less than 15% w/w of the ASBTI systemically. In certain embodiments, non-systemic compositions described herein deliver less than 10% w/w of the ASBTI systemically. In certain embodiments, non-systemic compositions described herein deliver less than 5% w/w of the ASBTI systemically. In some embodiments, systemic absorption is determined in any suitable manner, including the total circulating amount, the amount cleared after administration, or the like.

In certain embodiments, the compositions and/or formulations described herein are administered at least once a day. In certain embodiments, the formulations containing the ASBTI are administered at least twice a day, while in other embodiments the formulations containing the ASBTI are administered at least three times a day. In certain embodiments, the formulations containing the ASBTI are administered up to five times a day. It is to be understood that in certain embodiments, the dosage regimen of composition containing the ASBTI described herein to is determined by considering various factors such as the patient's age, sex, and diet.

The concentration of the ASBTI administered in the formulations described herein ranges from about 0.1 mM to about 1 M. In certain embodiments the concentration of the ASBTI administered in the formulations described herein ranges from about 1 mM to about 750 mM. In certain embodiments the concentration of the ASBTI administered in the formulations described herein ranges from about 1 mM to about 500 mM. In certain embodiments the concentration of the ASBTI administered in the formulations described herein ranges from about 1 mM to about 500 mM. In certain embodiments the concentration of the ASBTI administered in the formulations described herein ranges from about 1 mM to about 250 mM. In certain embodiments the concentration of the administered in the formulations described herein ranges from about 5 mM to about 100 mM. In certain embodiments the concentration of the ASBTI administered in the formulations described herein ranges from about 7 mM to about 70 mM. In certain embodiments the concentration of the ASBTI administered in the formulations described herein is about 7 mM, or about 10 mM, or about 15 mM, or about 20 mM, or about 25 mM, or about 30 mM, or about 40 mM, or about 50 mM, or about 60 mM, or about 70 mM.

In certain embodiments, by targeting the distal gastrointestinal tract (e.g., distal ileum, colon, and/or rectum), compositions and methods described herein provide efficacy (e.g., in reducing microbial growth and/or alleviating symptoms of cholestasis or a cholestatic liver disease) with a reduced dose of enteroendocrine peptide secretion enhancing agent (e.g., as compared to an oral dose that does not target the distal gastrointestinal tract).

In certain embodiments of the present disclosure, an effective amount of a given agent varies depending upon one or more of a number of factors such as the particular compound, disease or condition and its severity, the identity (e.g., weight) of the subject or host in need of treatment, and is determined according to the particular circumstances surrounding the case, including, e.g., the specific agent being administered, the route of administration, the condition being treated, and the subject or host being treated. In some embodiments, doses administered include those up to the maximum tolerable dose. In some embodiments, doses administered include those up to the maximum tolerable dose by a newborn or an infant.

In various embodiments of the present disclosure, a desired dose is conveniently presented in a single dose or in divided doses administered simultaneously (or over a short period of time) or at appropriate intervals, for example as two, three, four or more sub-doses per day. In various embodiments, a single dose of an ASBTI is administered every 6 hours, every 12 hours, every 24 hours, every 48 hours, every 72 hours, every 96 hours, every 5 days, every 6 days, or once a week. In some embodiments the total single dose of an ASBTI is in a range described below.

In various embodiments of the present disclosure, in the case wherein the patient's status does improve, upon the doctor's discretion an ASBTI is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100% of the original dose, including, by way of example only, 10%, 15%, 20%, 25%, 30%, 35%, 40%, 45%, 50%, 55%, 60%, 65%, 70%, 75%, 80%, 85%, 90%, 95%, or 100% of the original dose. In some embodiments the total single dose of an ASBTI is in a range described below.

Once improvement of the patient's conditions has occurred, a maintenance dose is administered if necessary. Subsequently, the dosage or the frequency of administration, or both, is reduced, as a function of the symptoms, to a level at which the improved disease, disorder or condition is retained. In some embodiments, patients require intermittent treatment on a long-term basis upon any recurrence of symptoms.

In certain instances, there are a large number of variables in regard to an individual treatment regime, and considerable excursions from these recommended values are considered within the scope described herein. Dosages described herein are optionally altered depending on a number of variables such as, by way of non-limiting example, the activity of the compound used, the disease or condition to be treated, the mode of administration, the requirements of the individual subject, the severity of the disease or condition being treated, and the judgment of the practitioner.

Toxicity and therapeutic efficacy of such therapeutic regimens are optionally determined by pharmaceutical procedures in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index and it can be expressed as the ratio between LD₅₀ and ED₅₀. Compounds exhibiting high therapeutic indices are prefer ed. In certain embodiments, data obtained from cell culture assays and animal studies are used in formulating a range of dosage for use in human. In specific embodiments, the dosage of compounds described herein lies within a range of circulating concentrations that include the ED₅₀ with minimal toxicity. The dosage optionally varies within this range depending upon the dosage form employed and the route of administration utilized.

Dosages

In various embodiments, the patient is a pediatric patient under the age of 1, 2, 3, 4, 5, 6, 7, 8, 9, 10, 11, 12, 13, 14, 15, 16, 17, or 18 years old. In certain embodiments, the pediatric subject is a newborn, a pre-term newborn, an infant, a toddler, a preschooler, a school-age child, a pre-pubescent child, post-pubescent child, an adolescent, or a teenager under the age of eighteen. In some embodiments, the pediatric subject is a newborn, a pre-term newborn, an infant, a toddler, a preschooler, or a school-age child. In some embodiments, the pediatric subject is a newborn, a pre-term newborn, an infant, a toddler, or a preschooler. In some embodiments, the pediatric subject is a newborn, a pre-term newborn, an infant, or a toddler. In some embodiments, the pediatric subject is a newborn, a pre-term newborn, or an infant. In some embodiments, the pediatric subject is a newborn. In some embodiments, the pediatric subject is an infant. In some embodiments, the pediatric subject is a toddler.

In various embodiments the ASBTI is maralixibat or volixibat, or a pharmaceutically acceptable salt thereof.

In various embodiments, efficacy and safety of ASBTI administration to the patient is monitored by measuring serum levels of 7α-hydroxy-4-cholesten-3-one (7αC4), sBA concentration, a ratio of 7αC4 to sBA (7αC4:sBA), serum total cholesterol concentration, serum LDL-C cholesterol concentration, serum bilirubin concentration, serum ALT concentration, serum AST concentration, or a combination thereof. In various embodiments, efficacy of ASBTI administration is measured by monitoring observer-reported itch reported outcome (ITCHRO(OBS)) score, a HRQoL (e.g., PedsQL) score, a CSS score, a xanthoma score, a height Z-score, a weight Z-score, or various combinations thereof. In various embodiments, the method includes monitoring serum levels of 7α-hydroxy-4-cholesten-3-one (7αC4), sBA concentration, a ratio of 7αC4 to sBA (7αC4:sBA), serum total cholesterol concentration, serum LDL-C cholesterol concentration, serum bilirubin concentration, serum ALT concentration, serum AST concentration, or a combination thereof. In various embodiments, the method includes monitoring observer-reported itch reported outcome (ITCHRO(OBS)) score, a HRQoL (e.g., PedsQL) score, a CSS score, a xanthoma score, a height Z-score, a weight Z-score, or various combinations thereof.

The administered dose of the ASBTI may be calculated based on the molecular weight of the ASBTI as the compound free base, or as the pharmaceutically acceptable salt. In one embodiment, the administered dose of the ASBTI is based on the compound as the pharmaceutically acceptable salt. In one embodiment, the administered dose of the ASBTI is based on the compound free base.

In some embodiments, the ASBTI is administered at a dose of about or at least about 0.5 μg/kg, 1 μg/kg, 2 μg/kg, 3 μg/kg, 4 μg/kg, 5 μg/kg, 6 μg/kg, 7 μg/kg, 8 μg/kg, 9 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 100 μg/kg, 140 μg/kg, 150 μg/kg, 200 μg/kg, 240 μg/kg, 280 μg/kg, 300 μg/kg, 250 μg/kg, 280 μg/kg, 300 μg/kg, 400 μg/kg, 500 μg/kg, 560 μg/kg, 600 μg/kg, 700 μg/kg, 800 μg/kg, 900 μg/kg, 1,000 μg/kg, 1,100 μg/kg, 1,200 μg/kg, 1,300 μg/kg, 1,400 μg/kg, 1500 μg/kg, 1,600 μg/kg, 1,700 μg/kg, 1,800 μg/kg, 1,900 μg/kg, or 2,000 μg/kg. In various embodiments, the ASBTI is administered at a dose not exceeding about 1 μg/kg, 2 μg/kg, 3 μg/kg, 4 μg/kg, 5 μg/kg, 6 μg/kg, 7 μg/kg, 8 μg/kg, 9 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 100 μg/kg, 140 μg/kg, 150 μg/kg, 200 μg/kg, 240 μg/kg, 280 μg/kg, 300 μg/kg, 250 μg/kg, 280 μg/kg, 300 μg/kg, 400 μg/kg, 500 μg/kg, 560 μg/kg, 600 μg/kg, 700 μg/kg, 800 μg/kg, 900 μg/kg, 1,000 μg/kg, 1,100 μg/kg, 1,200 μg/kg, 1,300 μg/kg, 1,400 μg/kg, 1,500 μg/kg, 1,600 μg/kg, 1,700 μg/kg, 1,800 μg/kg, 1,900 μg/kg, 2,000, or 2,100 μg/kg.

In various embodiments, the ASBTI is administered at a dose of about or of at least about 0.5 mg/day, 1 mg/day, 2 mg/day, 3 mg/day, 4 mg/day, 5 mg/day, 6 mg/day, 7 mg/day, 8 mg/day, 9 mg/day, 10 mg/day, 11 mg/day, 12 mg/day, 13 mg/day, 14 mg/day, 15 mg/day, 16 mg/day, 17 mg/day, 18 mg/day, 19 mg/day, 20 mg/day, 30 mg/day, 40 mg/day, 50 mg/day, 60 mg/day, 70 mg/day, 80 mg/day, 90 mg/day, 100 mg/day, 150 mg/day, 200 mg/day, 300 mg/day, 500 mg/day, 600 mg/day, 700 mg/day, 800 mg/day, 900 mg/day, 1000 mg/day. In various embodiments, the ASBTI is administered at a dose of not more than about 1 mg/day, 2 mg/day, 3 mg/day, 4 mg/day, 5 mg/day, 6 mg/day, 7 mg/day, 8 mg/day, 9 mg/day, 10 mg/day, 11 mg/day, 12 mg/day, 13 mg/day, 14 mg/day, 15 mg/day, 16 mg/day, 17 mg/day, 18 mg/day, 19 mg/day, 20 mg/day, 30 mg/day, 40 mg/day, 50 mg/day, 60 mg/day, 70 mg/day, 80 mg/day, 90 mg/day, 100 mg/day, 150 mg/day, 200 mg/day, 300 mg/day, 500 mg/day, 600 mg/day, 700 mg/day, 800 mg/day, 900 mg/day, 1,000 mg/day, 1,100 mg/day.

In some embodiments, the ASBTI is administered at a dose of from about 140 μg/kg/day to about 1400 μg/kg/day. In various embodiments, the ASBTI is administered at a dose of about or at least about 0.5 μg/kg/day, 1 μg/kg/day, 2 μg/kg/day, 3 μg/kg/day, 4 μg/kg/day, 5 μg/kg/day, 6 μg/kg/day, 7 μg/kg/day, 8 μg/kg/day, 9 μg/kg/day 10 μg/kg/day, 15 μg/kg/day, 20 μg/kg/day, 25 μg/kg/day, 30 μg/kg/day, 35 μg/kg/day, 40 μg/kg/day, 45 μg/kg/day, 50 μg/kg/day, 100 μg/kg/day, 140 μg/kg/day, 150 μg/kg/day, 200 μg/kg/day, 240 μg/kg/day, 280 μg/kg/day, 300 μg/kg/day, 250 μg/kg/day, 280 μg/kg/day, 300 μg/kg/day, 400 μg/kg/day, 500 μg/kg/day, 560 μg/kg/day, 600 μg/kg/day, 700 μg/kg/day, 800 μg/kg/day, 900 μg/kg/day, 1,000 μg/kg/day, 1,100 μg/kg/day, 1,200 μg/kg/day, or 1,300 μg/kg/day. In various embodiments, the ASBTI is administered at a dose not exceeding about 1 μg/kg/day, 2 μg/kg/day, 3 μg/kg/day, 4 μg/kg/day, 5 μg/kg/day, 6 μg/kg/day, 7 μg/kg/day, 8 μg/kg/day, 9 μg/kg/day 10 μg/kg/day, 15 μg/kg/day, 20 μg/kg/day, 25 μg/kg/day, 30 μg/kg/day, 35 μg/kg/day, 40 μg/kg/day, 45 μg/kg/day, 50 μg/kg/day, 100 μg/kg/day, 140 μg/kg/day, 150 μg/kg/day, 200 μg/kg/day, 240 μg/kg/day, 280 μg/kg/day, 300 μg/kg/day, 250 μg/kg/day, 280 μg/kg/day, 300 μg/kg/day, 360 μg/kg/day, 380 μg/kg/day, 400 μg/kg/day, 500 μg/kg/day, 560 μg/kg/day, 600 μg/kg/day, 700 μg/kg/day, 800 μg/kg/day, 880 μg/kg/day, 900 μg/kg/day, 1,000 μg/kg/day, 1,100 μg/kg/day, 1,200 μg/kg/day, 1,300 μg/kg/day, or 1,400 μg/kg/day. In various embodiments, the ASBTI is administered at a dose of from about 0.5 μg/kg/day to about 500 μg/kg/day, from about 0.5 μg/kg/day to about 250 μg/kg/day, from about 1 μg/kg/day to about 100 μg/kg/day, from about 10 μg/kg/day to about 50 μg/kg/day, from about 10 μg/kg/day to about 100 μg/kg/day, from about 0.5 μg/kg/day to about 2000 μg/kg/day, from about 280 μg/kg/day to about 1400 μg/kg/day, from about 420 μg/kg/day to about 1400 μg/kg/day, from about 250 to about 550 μg/kg/day, from about 560 μg/kg/day to about 1400 μg/kg/day, from 700 μg/kg/day to about 1400 μg/kg/day, from about 560 μg/kg/day to about 1200 μg/kg/day, from about 700 μg/kg/day to about 1200 μg/kg/day, from about 560 μg/kg/day to about 1000 μg/kg/day, from about 700 μg/kg/day to about 1000 μg/kg/day, from about 800 μg/kg/day to about 1000 μg/kg/day, from about 200 μg/kg/day to about 600 μg/kg/day, from about 300 μg/kg/day to about 600 μg/kg/day, from about 360 μg/kg/day to about 880 μg/kg/day, from about 400 μg/kg/day to about 500 μg/kg/day, from about 400 μg/kg/day to about 600 μg/kg/day, from about 400 μg/kg/day to about 700 μg/kg/day, from about 400 μg/kg/day to about 800 μg/kg/day, from about 500 μg/kg/day to about 800 μg/kg/day, from about 500 μg/kg/day to about 900 μg/kg/day, from about 600 μg/kg/day to about 900 μg/kg/day, from about 700 μg/kg/day to about 900 μg/kg/day, from about 200 μg/kg/day to about 600 μg/kg/day, from about 800 μg/kg/day to about 900 μg/kg/day, from about 100 μg/kg/day to about 1500 μg/kg/day, from about 300 μg/kg/day to about 2,000 μg/kg/day, or from about 400 μg/kg/day to about 2000 μg/kg/day.

In some embodiments, the ASBTI is administered at a dose of from about 30 μg/kg to about 1400 μg/kg per dose. In some embodiments, the ASBTI is administered at a dose of from about 0.5 μg/kg to about 2000 μg/kg per dose, from about 0.5 μg/kg to about 1500 μg/kg per dose, from about 100 μg/kg to about 700 μg/kg per dose, from about 5 μg/kg to about 100 μg/kg per dose, from about 10 μg/kg to about 500 μg/kg per dose, from about 50 μg/kg to about 1400 μg/kg per dose, from about 300 μg/kg to about 2,000 μg/kg per dose, from about 60 μg/kg to about 1200 μg/kg per dose, from about 70 μg/kg to about 1000 μg/kg per dose, from about 70 μg/kg to about 700 μg/kg per dose, from 80 μg/kg to about 1000 μg/kg per dose, from 80 μg/kg to about 800 μg/kg per dose, from 100 μg/kg to about 800 μg/kg per dose, from 100 μg/kg to about 600 μg/kg per dose, from 150 μg/kg to about 700 μg/kg per dose, from 150 μg/kg to about 500 μg/kg per dose, from 200 μg/kg to about 400 μg/kg per dose, from 200 μg/kg to about 300 μg/kg per dose, or from 300 μg/kg to about 400 μg/kg per dose.

In some embodiments, the ASBTI is administered at a dose of from about 0.5 mg/day to about 550 mg/day. In various embodiments, the ASBTI is administered at a dose of from about 1 mg/day to about 500 mg/day, from about 1 mg/day to about 300 mg/day, from about 1 mg/day to about 200 mg/day, from about 2 mg/day to about 300 mg/day, from about 2 mg/day to about 200 mg/day, from about 4 mg/day to about 300 mg/day, from about 4 mg/day to about 200 mg/day, from about 4 mg/day to about 150 mg/day, from about 5 mg/day to about 150 mg/day, from about 5 mg/day to about 100 mg/day, from about 5 mg/day to about 80 mg/day, from about 5 mg/day to about 50 mg/day, from about 5 mg/day to about 40 mg/day, from about 5 mg/day to about 30 mg/day, from about 5 mg/day to about 20 mg/day, from about 5 mg/day to about 15 mg/day, from about 10 mg/day to about 100 mg/day, from about 10 mg/day to about 80 mg/day, from about 10 mg/day to about 50 mg/day, from about 10 mg/day to about 40 mg/day, from about 10 mg/day to about 20 mg/day, from about 20 mg/day to about 100 mg/day, from about 20 mg/day to about 80 mg/day, from about 20 mg/day to about 50 mg/day, or from about 20 mg/day to about 40 mg/day, or from about 20 mg/day to about 30 mg/day.

In some embodiments, the ASBTI is administered twice daily (BID) in an amount of about 200 μg/kg to about 400 μg/kg per dose. In some embodiments, the ASBTI is administered in an amount of about 280 μg/kg/day to about 1400 μg/kg/day. In some embodiments, the ASBTI is administered in an amount of about 400 μg/kg/day to about 800 μg/kg/day. In some embodiments, the ASBTI is administered in an amount of about 360 μg/kg/day to about 880 μg/kg/day. In some embodiments, the ASBTI is administered in an amount of about 20 mg/day to about 50 mg/day. In some embodiments, the ASBTI is administered in an amount of from about 5 mg/day to about 15 mg/day. In some embodiments, the ASBTI is administered in an amount of from about 560 μg/kg/day to about 1,400 μg/kg/day. In some embodiments, the ASBTI is administered in an amount of from about 700 μg/kg/day to about 1,400 μg/kg/day. In some embodiments, the ASBTI is administered in an amount of from about 400 μg/kg/day to about 800 μg/kg/day. In some embodiments, the ASBTI is administered in an amount of from about 700 μg/kg/day to about 900 μg/kg/day. In some embodiments, the ASBTI is administered in an amount of from about 560 μg/kg/day to about 1400 μg/kg/day. In some embodiments, the ASBTI is administered in an amount from 700 μg/kg/day to about 1400 μg/kg/day. In some embodiments, the ASBTI is administered in an amount of from about 200 μg/kg/day to about 600 μg/kg/day. In some embodiments, the ASBTI is administered in an amount of from about 400 μg/kg/day to about 600 μg/kg/day.

In various embodiments, the dose of the ASBTI is a first dose level. In various embodiments, the dose of the ASBTI is a second dose level. In some embodiments, the second dose level is greater than the first dose level. In some embodiments, the second dose level is about or at least about 1.5, 2, 3, 4, 5, 6, 7, 8, 9, 10, 15, 20, 30, 40, 50, 60, 70, 80, 90 or 100 times or fold greater than the first dose level. In some embodiments, the second dose level is not in excess of about 1.5, 2, 3, 4, 5, 6, 7, 8, 9, 10, 15, 20, 30, 40, 50, 60, 70, 80, 90, 100, or 150 times or fold greater than the first dose level.

In various embodiments, the ASBTI is administered once daily (QD) at one of the above doses or within one of the above dose ranges. In various embodiments, the ASBTI is administered twice daily (BID) at one of the above doses or within one of the above dose ranges. In various embodiments, an ASBTI dose is administered daily, every other day, twice a week, or once a week.

In various embodiments, the ASBTI is administered regularly for a period of about or of at least about 1, 2, 3, 4, 5, 6, 7, 8, 9, 10, 15, 20, 30, 40, 48, 50, 75, 100, 150, 200, 250, 300, 350, 400, 450, 500, 600, 700, or 800 weeks. In various embodiments, the ASBTI is administered for not more than about 1, 2, 3, 4, 5, 6, 7, 8, 9, 10, 15, 20, 30, 40, 48, 50, 75, 100, 150, 200, 250, 300, 350, 400, 450, 500, 600, 700, 800, or 1000 weeks. In various embodiments, the ASBTI is administered regularly for a period of about or of at least about 0.5, 1, 1.5, 2, 3, 4, 5, 6, 7, 8, 9, or 10 years. In various embodiments, the ASBTI is administered regularly for a period not in excess of about 0.5, 1, 1.5, 2, 3, 4, 5, 6, 7, 8, 9, 10, or 15 years.

Oral Administration for Terminal Ileum or Colonic Delivery

In certain aspects, the composition or formulation containing one or more compounds described herein is orally administered for local delivery of an ASBTI, or a compound described herein to the terminal ileum, colon and/or rectum. Unit dosage forms of such compositions include liquid dosage forms formulated for enteric delivery to the terminal ileum and/or colon. In certain embodiments, such liquid dosage forms, e.g., solutions, suspensions, or elixirs, contain the compositions described herein entrapped or embedded in microspheres. In some embodiments, microspheres include, by way of non-limiting example, chitosan microcores HPMC capsules and cellulose acetate butyrate (CAB) microspheres. In certain embodiments, oral dosage forms are prepared using conventional methods known to those in the field of pharmaceutical formulation.

In some embodiments, ASBTIs as described herein are orally administered in association with a carrier suitable for delivery to the distal gastrointestinal tract (e.g., distal and/or terminal ileum, colon, and/or rectum).

In certain embodiments, a composition described herein comprises an ASBTI, or other compounds described herein in association with a matrix (e.g., a matrix comprising hypermellose) that allows for controlled release of an active agent in the distal part of the ileum and/or the colon. In some embodiments, a composition comprises a polymer that is pH sensitive (e.g., a MMX™ matrix from Cosmo Pharmaceuticals) and allows for controlled release of an active agent in the distal part of the ileum. Examples of such pH sensitive polymers suitable for controlled release include and are not limited to polyacrylic polymers (e.g., anionic polymers of methacrylic acid and/or methacrylic acid esters, e.g., Carbopol® polymers) that comprise acidic groups (e.g., —COOH, —SO3H) and swell in basic pH of the intestine (e.g., pH of about 7 to about 8). In some embodiments, a composition suitable for controlled release in the distal ileum comprises microparticulate active agent (e.g., micronized active agent). In some embodiments, a non-enzymatically degrading poly(dl-lactide-co-glycolide) (PLGA) core is suitable for delivery of an enteroendocrine peptide secretion enhancing agent to the distal ileum. In some embodiments, a dosage form comprising an enteroendocrine peptide secretion enhancing agent is coated with an enteric polymer (e.g., Eudragit® S-100, cellulose acetate phthalate, polyvinylacetate phthalate, hydroxypropylmethylcellulose phthalate, anionic polymers of methacrylic acid, methacrylic acid esters or the like) for site specific delivery to the distal ileum and/or the colon. In some embodiments, bacterially activated systems are suitable for targeted delivery to the distal part of the ileum. Examples of micro-flora activated systems include dosage forms comprising pectin, galactomannan, and/or Azo hydrogels and/or glycoside conjugates (e.g., conjugates of D-galactoside, β-D-xylopyranoside or the like) of the active agent. Examples of gastrointestinal micro-flora enzymes include bacterial glycosidases such as, for example, D-galactosidase, β-D-glucosidase, α-L-arabinofuranosidase, β-D-xylopyranosidase or the like.

The pharmaceutical compositions described herein optionally include an additional therapeutic compound described herein and one or more pharmaceutically acceptable additives such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof.

Bile Acid Sequestrant

In certain embodiments, a composition described herein is, e.g., an ASBTI in association with a labile bile acid sequestrant. A labile bile acid sequestrant is a bile acid sequestrant with a labile affinity for bile acids. In certain embodiments, a bile acid sequestrant described herein is an agent that sequesters (e.g., absorbs or is charged with) bile acid, and/or the salts thereof.

In specific embodiments, the labile bile acid sequestrant is an agent that sequesters (e.g., absorbs or is charged with) bile acid, and/or the salts thereof, and releases at least a portion of the absorbed or charged bile acid, and/or salts thereof in the distal gastrointestinal tract (e.g., the colon, ascending colon, sigmoid colon, distal colon, rectum, or any combination thereof). In certain embodiments, the labile bile acid sequestrant is an enzyme dependent bile acid sequestrant. In specific embodiments, the enzyme is a bacterial enzyme. In some embodiments, the enzyme is a bacterial enzyme found in high concentration in human colon or rectum relative to the concentration found in the small intestine. Examples of micro-flora activated systems include dosage forms comprising pectin, galactomannan, and/or Azo hydrogels and/or glycoside conjugates (e.g., conjugates of D-galactoside, β-D-xylopyranoside or the like) of the active agent. Examples of gastrointestinal micro-flora enzymes include bacterial glycosidases such as, for example, D-galactosidase, β-D-glucosidase, α-L-arabinofuranosidase, β-D-xylopyranosidase or the like. In some embodiments, the labile bile acid sequestrant is a time dependent bile acid sequestrant (i.e., the bile acid sequesters the bile acid and/or salts thereof and after a time releases at least a portion of the bile acid and/or salts thereof). In some embodiments, a time dependent bile acid sequestrant is an agent that degrades in an aqueous environment over time. In certain embodiments, a labile bile acid sequestrant described herein is a bile acid sequestrant that has a low affinity for bile acid and/or salts thereof, thereby allowing the bile acid sequestrant to continue to sequester bile acid and/or salts thereof in an environ where the bile acids/salts and/or salts thereof are present in high concentration and release them in an environ wherein bile acids/salts and/or salts thereof are present in a lower relative concentration. In some embodiments, the labile bile acid sequestrant has a high affinity for a primary bile acid and a low affinity for a secondary bile acid, allowing the bile acid sequestrant to sequester a primary bile acid or salt thereof and subsequently release a secondary bile acid or salt thereof as the primary bile acid or salt thereof is converted (e.g., metabolized) to the secondary bile acid or salt thereof. In some embodiments, the labile bile acid sequestrant is a pH dependent bile acid sequestrant. In some embodiments, the pH dependent bile acid sequestrant has a high affinity for bile acid at a pH of 6 or below and a low affinity for bile acid at a pH above 6. In certain embodiments, the pH dependent bile acid sequestrant degrades at a pH above 6.

In some embodiments, labile bile acid sequestrants described herein include any compound, e.g., a macro-structured compound, that can sequester bile acids/salts and/or salts thereof through any suitable mechanism. For example, in certain embodiments, bile acid sequestrants sequester bile acids/salts and/or salts thereof through ionic interactions, polar interactions, static interactions, hydrophobic interactions, lipophilic interactions, hydrophilic interactions, steric interactions, or the like. In certain embodiments, macrostructured compounds sequester bile acids/salts and/or sequestrants by trapping the bile acids/salts and/or salts thereof in pockets of the macrostructured compounds and, optionally, other interactions, such as those described above. In some embodiments, bile acid sequestrants (e.g., labile bile acid sequestrants) include, by way of non-limiting example, lignin, modified lignin, polymers, polycationic polymers and copolymers, polymers and/or copolymers comprising anyone one or more of N-alkenyl-N-alkylaminc residues; one or more N,N,N-trialkyl-N—(N′-alkenylamino)alkyl-azanium residues; one or more N,N,N-trialkyl-N-alkenyl-azanium residues; one or more alkenyl-amine residues; or a combination thereof, or any combination thereof.

Covalent Linkage of the Drug with a Carrier

In some embodiments, strategies used for colon targeted delivery include, by way of non-limiting example, covalent linkage of the ASBTI or other compounds described herein to a carrier, coating the dosage form with a pH-sensitive polymer for delivery upon reaching the pH environment of the colon, using redox sensitive polymers, using a time released formulation, utilizing coatings that are specifically degraded by colonic bacteria, using bioadhesive system and using osmotically controlled drug delivery systems.

In certain embodiments of such oral administration of a composition containing an ASBTI or other compounds described herein involves covalent linking to a carrier wherein upon oral administration the linked moiety remains intact in the stomach and small intestine. Upon entering the colon, the covalent linkage is broken by the change in pH, enzymes, and/or degradation by intestinal microflora. In certain embodiments, the covalent linkage between the ASBTI and the carrier includes, by way of non-limiting example, azo linkage, glycoside conjugates, glucuronide conjugates, cyclodextrin conjugates, dextran conjugates, and amino-acid conjugates (high hydrophilicity and long chain length of the carrier amino acid).

Coating with Polymers: pH-Sensitive Polymers

In some embodiments, the oral dosage forms described herein are coated with an enteric coating to facilitate the delivery of an ASBTI or other compounds described herein to the colon and/or rectum. In certain embodiments, an enteric coating is one that remains intact in the low pH environment of the stomach, but readily dissolved when the optimum dissolution pH of the particular coating is reached which depends upon the chemical composition of the enteric coating. The thickness of the coating will depend upon the solubility characteristics of the coating material. In certain embodiments, the coating thicknesses used in such formulations described herein range from about 25 μm to about 200 μm.

In certain embodiments, the compositions or formulations described herein are coated such that an ASBTI or other compounds described herein of the composition or formulation is delivered to the colon and/or rectum without absorbing at the upper part of the intestine. In a specific embodiment, specific delivery to the colon and/or rectum is achieved by coating of the dosage form with polymers that degrade only in the pH environment of the colon. In alternative embodiments, the composition is coated with an enteric coat that dissolves in the pH of the intestines and an outer layer matrix that slowly erodes in the intestine. In some of such embodiments, the matrix slowly erodes until only a core composition comprising an enteroendocrine peptide secretion enhancing agent (and, in some embodiments, an absorption inhibitor of the agent) is left and the core is delivered to the colon and/or rectum.

In certain embodiments, pH-dependent systems exploit the progressively increasing pH along the human gastrointestinal tract (GIT) from the stomach (pH 1-2 which increases to 4 during digestion), small intestine (pH 6-7) at the site of digestion and it to 7-8 in the distal ileum. In certain embodiments, dosage forms for oral administration of the compositions described herein are coated with pH-sensitive polymer(s) to provide delayed release and protect the enteroendocrine peptide secretion enhancing agents from gastric fluid. In certain embodiments, such polymers are be able to withstand the lower pH values of the stomach and of the proximal part of the small intestine but disintegrate at the neutral or slightly alkaline pH of the terminal ileum and/or ileocecal junction. Thus, in certain embodiments, provided herein is an oral dosage form comprising a coating, the coating comprising a pH-sensitive polymer. In some embodiments, the polymers used for colon and/or rectum targeting include, by way of non-limiting example, methacrylic acid copolymers, methacrylic acid and methyl methacrylate copolymers, Eudragit L100, Eudragit S100, Eudragit L-30D, Eudragit FS-30D, Eudragit L100-55, polyvinylacetate phthalate, hyrdoxypropyl ethyl cellulose phthalate, hyrdoxypropyl methyl cellulose phthalate 50, hyrdoxypropyl methyl cellulose phthalate 55, cellulose acetate trimelliate, cellulose acetate phthalate and combinations thereof.

In certain embodiments, oral dosage forms suitable for delivery to the colon and/or rectum comprise a coating that has a biodegradable and/or bacteria degradable polymer or polymers that are degraded by the microflora (bacteria) in the colon. In such biodegradable systems suitable polymers include, by way of non-limiting example, azo polymers, linear-type-segmented polyurethanes containing azo groups, polygalactomannans, pectin, glutaraldehyde crosslinked dextran, polysaccharides, amylose, guar gum, pectin, chitosan, inulin, cyclodextrins, chondroitin sulphate, dextrans, locust bean gum, chondroitin sulphate, chitosan, poly (-caprolactone), polylactic acid and poly(lactic-co-glycolic acid).

In certain embodiments of such oral administration of compositions containing one or more ASBTIs or other compounds described herein, the compositions are delivered to the colon without absorbing at the upper part of the intestine by coating of the dosage forms with redox sensitive polymers that are degraded by the microflora (bacteria) in the colon. In such biodegradable systems such polymers include, by way of non-limiting example, redox-sensitive polymers containing an azo and/or a disulfide linkage in the backbone.

In some embodiments, compositions formulated for delivery to the colon and/or rectum are formulated for time-release. In some embodiments, time release formulations resist the acidic environment of the stomach, thereby delaying the release of the enteroendocrine peptide secretion enhancing agents until the dosage form enters the colon and/or rectum.

Combination Therapy

In clinical practice, the majority of patients with ALGS are treated with off-label agents, most commonly UDCA and rifampicin, to control or reduce pruritus symptoms. These medications are usually only partially or temporarily effective in reducing the pruritus associated with cholestatic liver disease, such as ALGS or PFIC.

In some embodiments, the compositions described herein are administered in combination with one or more additional agents. In some embodiments, the present invention also provides a composition comprising a compound (e.g., an ASBTI) with one or more additional agents. In some embodiments, a reduction in amount/dosing of the ASBTI and/or the second therapeutic agent is achieved, as compared to the amount/dosing of the ASBTI and/or the second therapeutic agent administered as a monotherapy.

In some embodiments, a reduction in amount/dosing of the second therapeutic agent is achieved. In some embodiments, a reduction in amount/dosing of the second therapeutic agent by at least 10%, or at least 15%, or at least 20%, or at least 25%, or at least 30%, or at least 35%, or at least 40%, or at least 50%, or at least 60%, or at least 70%, or at least 75%, or at least 80%, or at least 90% as compared to an amount/dosing of the second therapeutic agent administered as a monotherapy is achieved.

In some embodiments, the subject is able to discontinue the therapy with the second therapeutic agent, i.e., a 100% reduction in the amount/dosing of the second therapeutic agent is achieved.

In some embodiments, the compositions described herein comprise a combination of an ASBTI (e.g., maralixibat) with a subclinical therapeutically effective amount of a second therapeutic agent selected from the group consisting of UDCA, rifampicin, an antihistamine, and an FXR-targeting drug.

In some embodiments, the compositions of ASBTIs described herein are administered in combination with a subclinical therapeutically effective amount of a second therapeutic agent selected from the group consisting of UDCA, rifampicin, an antihistamine, and an FXR-targeting drug.

Fat Soluble Vitamins

In some embodiments, the compositions provided herein further comprise one or more vitamins. In some embodiments, the vitamin is vitamin A, B1, B2, B3, B5, B6, B7, B9, B12, C, D, E, K, folic acid, pantothenic acid, niacin, riboflavin, thiamine, retinol, beta carotene, pyridoxine, ascorbic acid, cholecalciferol, cyanocobalamin, tocopherols, phylloquinone, menaquinone.

In some embodiments, the vitamin is a fat soluble vitamin such as vitamin A, D, E, K, retinol, beta carotene, cholecalciferol, tocopherols, phylloquinone. In a preferred embodiment, the fat soluble vitamin is tocopherol polyethylene glycol succinate (TPGS).

Partial External Biliary Diversion (PEBD)

In some embodiments, the methods of use of the compositions provided herein further comprise using partial external biliary diversion as a treatment for patients who have not yet developed cirrhosis. This treatment helps reduce the circulation of bile acids/salts in the liver in order to reduce complications and prevent the need for early transplantation in many patients.

This surgical technique involves isolating a segment of intestine 10 cm long for use as a biliary conduit (a channel for the passage of bile) from the rest of the intestine. One end of the conduit is attached to the gallbladder and the other end is brought out to the skin to form a stoma (a surgically constructed opening to permit the passage of waste). Partial external biliary diversion may be used for patients who are unresponsive to all medical therapy, especially older, larger patients. This procedure may not be of help to young patients such as infants. Partial external biliary diversion may decrease the intensity of the itching and abnormally low levels of cholesterol in the blood.

ASBTIs and PPAR Agonists

In various embodiments, the present disclosure provides combinations of ASBTIs with PPAR (peroxisome proliferator-activated receptor) agonists. In various embodiments, the PPAR agonist is a fibrate drug. In some embodiments, the fibrate drug is clofibrate, gemfibrozil, ciprofibrate, benzafibrate, fenofibrate, or various combinations thereof. In various embodiments, the PPAR agonist is aleglitazar, muraglitazar, tesaglitazar, saroglitazar, GW501516, GW-9662, a thiazolidinedione (TZD), a NSAID (e.g., IBUPROFEN), an indole, or various combinations thereof. In some embodiments, the PPAR agonist is bezafibrate, seladelpar (MBX-8025), GW501516 (Cardarine), fenofibrate, elafibranor, REN001, KD3010, ASP0367, or CER-002.

In various embodiments, the PPAR agonist used in combinations with ASBTIs of the present disclosure is a pan-PPAR agonist, or a PPARα, a PPARγ, a PPARβ, or a PPARδ agonist.

In one non-limited embodiment, the PPAR agonist is a PPARδ agonist. In one embodiment, the PPARδ agonist is seladelpar (MBX-8025), GW501516 (Cardarine), REN001, KD3010, ASP0367, or CER-002.

ASBTIs and FXR Drugs

In various embodiments, the present disclosure provides combinations of ASBTIs with farnesoid X receptor (FXR) targeting drugs. In various embodiments, the FXR targeting drug is avermectin B1a, bepridil, fluticasone propionate, GW4064, gliquidone, nicardipine, triclosan, CDCA, ivermectin, chlorotrianisene, tribenoside, mometasone furoate, miconazole, amiodarone, butoconazolee, bromocryptine mesylate, pizotifen malate, or various combinations thereof. In some embodiments, a reduction in amount/dosing of the ASBTI and/or the FXR-targeting drug is achieved, as compared to the amount/dosing of the ASBTI and/or the FXR-targeting drug administered as a monotherapy.

ASBTIs and Antihistamines

In various embodiments, the present disclosure provides combinations of ASBTIs with an antihistamine. In various embodiments, the antihistamine is azelastine, carbinoxamine, cyproheptadine, desloratadine, emedastine, hydroxyzine, levocabastine, levocetirizine, brompheniramine, cetirizine, chlorpheniramine, clemastine, diphenhydramine, fexofenadine, loratidine, or various combinations thereof. In some embodiments, a reduction in amount/dosing of the ASBTI and/or the antihistamine is achieved, as compared to the amount/dosing of the ASBTI and/or the antihistamine administered as a monotherapy.

ASBTI and Ursodiol UDCA

In some embodiments, the disclosed compositions are administered in combination with ursodiol or ursodeoxycholic acid (UDCA), chenodeoxycholic acid, cholic acid, taurocholic acid, ursocholic acid, glycocholic acid, glycodeoxycholic acid, taurodeoxycholic acid, taurocholate, glycochenodeoxycholic acid, tauroursodeoxycholic acid. In some embodiments, an increase in the concentration of bile acids/salts in the distal intestine induces intestinal regeneration, attenuating intestinal injury, reducing bacterial translocation, inhibiting the release of free radical oxygen, inhibiting production of proinflammatory cytokines, or any combination thereof or any combination thereof.

In certain embodiments, the patient is administered ursodiol at a daily dose of about or of at least about 5 mg, 10 mg, 15 mg, 20 mg, 25 mg, 30 mg, 35 mg, 36 mg, 40 mg, 45 mg, 50 mg, 55 mg, 60 mg, 65 mg, 70 mg, 75 mg, 80 mg, 85 mg, 90 mg, 95 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1,000 mg, 1,250 mg, 1,500 mg, 1,750 mg, 2,000 mg, 2,250 mg, 2,500 mg, 2,750 mg, or 3,000 mg. In certain embodiments, the patient is administered ursodiol at a daily dose of about or of no more than about 10 mg, 15 mg, 20 mg, 25 mg, 30 mg, 35 mg, 36 mg, 40 mg, 45 mg, 50 mg, 55 mg, 60 mg, 65 mg, 70 mg, 75 mg, 80 mg, 85 mg, 90 mg, 95 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1,000 mg, 1,250 mg, 1,500 mg, 1,750 mg, 2,000 mg, 2,250 mg, 2,500 mg, 2,750 mg, 3,000 mg, or 3,500 mg. In various embodiments, the patient is administered ursodiol at a daily dose of about or of at least about 3 mg to about 300 mg, about 30 mg to about 250 mg, from about 36 mg to about 200 mg, from about 10 mg to about 3000 mg, from about 1000 mg to about 2000 mg, or from about 1500 to about 1900 mg.

In various embodiments the ursodiol is administered as a tablet. In various embodiments, the ursodiol is administered as a suspension. In various embodiments, the concentration of ursodiol in the suspension is from about 10 mg/mL to about 200 mg/mL, from about 50 mg/mL to about 150 mg/mL, from about 10 mg/mL to about 500 mg/mL, or from about 40 mg/mL to about 60 mg/mL. In various embodiments, the concentration of ursodiol in suspension is about or is at least about 20 mg/mL, 25 mg/mL, 30 mg/mL, 35 mg/mL, 40 mg/mL, 45 mg/mL, 50 mg/mL, 55 mg/mL, 60 mg/mL, 65 mg/mL, 70 mg/mL, 75 mg/mL, or 80 mg/mL. In various embodiments, the concentration of ursodiol in suspension is no more than about 25 mg/mL, 30 mg/mL, 35 mg/mL, 40 mg/mL, 45 mg/mL, 50 mg/mL, 55 mg/mL, 60 mg/mL, 65 mg/mL, 70 mg/mL, 75 mg/mL, 80 mg/mL, or 85 mg/mL.

In certain embodiments, the patient is administered UDCA at a daily dose of about or of at least about 5 mg, 10 mg, 15 mg, 20 mg, 25 mg, 30 mg, 35 mg, 36 mg, 40 mg, 45 mg, 50 mg, 55 mg, 60 mg, 65 mg, 70 mg, 75 mg, 80 mg, 85 mg, 90 mg, 95 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1,000 mg, 1,250 mg, 1,500 mg, 1,750 mg, 2,000 mg, 2,250 mg, 2,500 mg, 2,750 mg, or 3,000 mg. In certain embodiments, the patient is administered UDCA at a daily dose of about or of no more than about 10 mg, 15 mg, 20 mg, 25 mg, 30 mg, 35 mg, 36 mg, 40 mg, 45 mg, 50 mg, 55 mg, 60 mg, 65 mg, 70 mg, 75 mg, 80 mg, 85 mg, 90 mg, 95 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1,000 mg, 1,250 mg, 1,500 mg, 1,750 mg, 2,000 mg, 2,250 mg, 2,500 mg, 2,750 mg, 3,000 mg, or 3,500 mg. In various embodiments, the patient is administered UDCA at a daily dose of about or of at least about 3 mg to about 300 mg, about 30 mg to about 250 mg, from about 36 mg to about 200 mg, from about 10 mg to about 3000 mg, from about 1000 mg to about 2000 mg, or from about 1500 to about 1900 mg.

In various embodiments the UDCA is administered as a tablet. In various embodiments, the UDCA is administered as a suspension. In various embodiments, the concentration of UDCA in the suspension is from about 10 mg/mL to about 200 mg/mL, from about 50 mg/mL to about 150 mg/mL, from about 10 mg/mL to about 500 mg/mL, or from about 40 mg/mL to about 60 mg/mL. In various embodiments, the concentration of UDCA in suspension is about or is at least about 20 mg/mL, 25 mg/mL, 30 mg/mL, 35 mg/mL, 40 mg/mL, 45 mg/mL, 50 mg/mL, 55 mg/mL, 60 mg/mL, 65 mg/mL, 70 mg/mL, 75 mg/mL, or 80 mg/mL. In various embodiments, the concentration of UDCA in suspension is no more than about 25 mg/mL, 30 mg/mL, 35 mg/mL, 40 mg/mL, 45 mg/mL, 50 mg/mL, 55 mg/mL, 60 mg/mL, 65 mg/mL, 70 mg/mL, 75 mg/mL, 80 mg/mL, or 85 mg/mL.

An ASBTI and a second active ingredient are used such that the combination is present in a therapeutically effective amount. That therapeutically effective amount arises from the use of a combination of an ASBTI and the other active ingredient (e.g., ursodiol or UDCA) wherein each is used in a therapeutically effective amount, or by virtue of additive or synergistic effects arising from the combined use, each can also be used in a subclinical therapeutically effective amount, i.e., an amount that, if used alone, provides for reduced effectiveness for the therapeutic purposes noted herein, provided that the combined use is therapeutically effective. In some embodiments, the use of a combination of an ASBTI and any other active ingredient as described herein encompasses combinations where the ASBTI or the other active ingredient is present in a therapeutically effective amount, and the other is present in a subclinical therapeutically effective amount, provided that the combined use is therapeutically effective owing to their additive or synergistic effects. As used herein, the term “additive effect” describes the combined effect of two (or more) pharmaceutically active agents that is equal to the sum of the effect of each agent given alone. A synergistic effect is one in which the combined effect of two (or more) pharmaceutically active agents is greater than the sum of the effect of each agent given alone. Any suitable combination of an ASBTI with one or more of the aforementioned other active ingredients and optionally with one or more other pharmacologically active substances is contemplated as being within the scope of the methods described herein.

In some embodiments, a reduction in amount/dosing of the ASBTI and/or UDCA is achieved, as compared to the amount/dosing of the ASBTI and/or UDCA administered as a monotherapy.

In some embodiments, the particular choice of compounds depends upon the diagnosis of the attending physicians and their judgment of the condition of the individual and the appropriate treatment protocol. The compounds are optionally administered concurrently (e.g., simultaneously, essentially simultaneously or within the same treatment protocol) or sequentially, depending upon the nature of the disease, disorder, or condition, the condition of the individual, and the actual choice of compounds used. In certain instances, the determination of the order of administration, and the number of repetitions of administration of each therapeutic agent during a treatment protocol, is based on an evaluation of the disease being treated and the condition of the individual.

In some embodiments, therapeutically-effective dosages vary when the drugs are used in treatment combinations. Methods for experimentally determining therapeutically-effective dosages of drugs and other agents for use in combination treatment regimens are described in the literature.

In some embodiments of the combination therapies described herein, dosages of the co-administered compounds vary depending on the type of co-drug employed, on the specific drug employed, on the disease or condition being treated and so forth. In addition, when co-administered with one or more biologically active agents, the compound provided herein is optionally administered either simultaneously with the biologically active agent(s), or sequentially. In certain instances, if administered sequentially, the attending physician will decide on the appropriate sequence of therapeutic compound described herein in combination with the additional therapeutic agent.

The multiple therapeutic agents (at least one of which is a therapeutic compound described herein) are optionally administered in any order or even simultaneously. If simultaneously, the multiple therapeutic agents are optionally provided in a single, unified form, or in multiple forms (by way of example only, either as a single pill or as two separate pills). In certain instances, one of the therapeutic agents is optionally given in multiple doses. In other instances, both are optionally given as multiple doses. If not simultaneous, the timing between the multiple doses is any suitable timing; e.g., from more than zero weeks to less than four weeks. In addition, the combination methods, compositions and formulations are not to be limited to the use of only two agents; the use of multiple therapeutic combinations are also envisioned (including two or more compounds described herein).

In certain embodiments, a dosage regimen to treat, prevent, or ameliorate the condition(s) for which relief is sought, is modified in accordance with a variety of factors. These factors include the disorder from which the subject suffers, as well as the age, weight, sex, diet, and medical condition of the subject. Thus, in various embodiments, the dosage regimen actually employed varies and deviates from the dosage regimens set forth herein.

In some embodiments, the pharmaceutical agents which make up the combination therapy described herein are provided in a combined dosage form or in separate dosage forms intended for substantially simultaneous administration. In certain embodiments, the pharmaceutical agents that make up the combination therapy are administered sequentially, with either therapeutic compound being administered by a regimen calling for two-step administration. In some embodiments, two-step administration regimen calls for sequential administration of the active agents or spaced-apart administration of the separate active agents. In certain embodiments, the time period between the multiple administration steps varies, by way of non-limiting example, from a few minutes to several hours, depending upon the properties of each pharmaceutical agent, such as potency, solubility, bioavailability, plasma half-life and kinetic profile of the pharmaceutical agent.

In certain embodiments, provided herein are combination therapies. In certain embodiments, the compositions described herein comprise an additional therapeutic agent. In some embodiments, the methods described herein comprise administration of a second dosage form comprising an additional therapeutic agent. In certain embodiments, combination therapies the compositions described herein are administered as part of a regimen. Therefore, additional therapeutic agents and/or additional pharmaceutical dosage form can be applied to a patient either directly or indirectly, and concomitantly or sequentially, with the compositions and formulations described herein.

Kits

In another aspect, provided herein are kits containing a device for administration pre-filled with a pharmaceutical composition described herein. In certain embodiments, kits contain a device for oral administration and a pharmaceutical composition as described herein. In certain embodiments the kits include prefilled sachet or bottle for oral administration, while in other embodiments the kits include prefilled bags for administration of gels. In certain embodiments the kits include prefilled syringes for administration of oral enemas.

In some embodiments, the kits include a bottle with a pre-instralled adapter and a child-resistant cap. In some embodiments, the bottle may have a volume of 10 mL, or 20 mL, or 30 mL, or 40 mL, or 50 mL, or 60 mL, or 80 mL, or 100 mL, or 200 mL, or 250 mL.

In some embodiments, the kits include one or more oral dosing dispensers, e.g., oral syringes. In some embodiments, the oral syringes may have a volume of 0.1 mL, or 0.2 mL, or 0.25 mL, or 0.5 mL, or 1 mL, or 2 mL, or 3 mL, or 5 mL, or 10 mL.

In one non-limiting embodiment, the kit includes a bottle having a volume of 30 mL and three oral syringes having volumes of 0.5 mL, 1 mL, and 3 mL co-packaged in a secondary container closure system.

Release in Distal Ileum and/or Colon

In certain embodiments, a composition and/or a dosage form comprises a matrix (e.g., a matrix comprising hypermellose) that allows for controlled release of an active agent in the distal jejunum, proximal ileum, distal ileum and/or the colon. In some embodiments, a composition and/or a dosage form comprises a polymer that is pH sensitive (e.g., a MMX™ matrix from Cosmo Pharmaceuticals) and allows for controlled release of an active agent in the ileum and/or the colon. Examples of such pH sensitive polymers suitable for controlled release include and are not limited to polyacrylic polymers (e.g., anionic polymers of methacrylic acid and/or methacrylic acid esters, e.g., Carbopol® polymers) that comprise acidic groups (e.g., —COOH, —SO3H) and swell in basic pH of the intestine (e.g., pH of about 7 to about 8). In some embodiments, a composition and/or a dosage form suitable for controlled release in the distal ileum comprises microparticulate active agent (e.g., micronized active agent). In some embodiments, a non-enzymatically degrading poly(dl-lactide-co-glycolide) (PLGA) core is suitable for delivery of an ASBTI to the distal ileum. In some embodiments, a dosage form comprising an ASBTI is coated with an enteric polymer (e.g., Eudragit® S-100, cellulose acetate phthalate, polyvinylacetate phthalate, hydroxypropylmethylcellulose phthalate, anionic polymers of methacrylic acid, methacrylic acid esters or the like) for site specific delivery to the ileum and/or the colon. In some embodiments, bacterially activated systems are suitable for targeted delivery to the ileum. Examples of micro-flora activated systems include dosage forms comprising pectin, galactomannan, and/or Azo hydrogels and/or glycoside conjugates (e.g., conjugates of D-galactoside, β-D-xylopyranoside or the like) of the active agent. Examples of gastrointestinal micro-flora enzymes include bacterial glycosidases such as, for example, D-galactosidase, β-D-glucosidase, α-L-arabinofuranosidase, β-D-xylopyranosidase or the like.

The pharmaceutical compositions and/or dosage forms described herein optionally include an additional therapeutic compound described herein and one or more pharmaceutically acceptable additives such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof. In some aspects, using standard coating procedures, such as those described in Remington's Pharmaceutical Sciences, 20th Edition (2000), a film coating is provided around the formulation of the ASBTI. In one embodiment, a compound described herein is in the form of a particle and some or all of the particles of the compound are coated. In certain embodiments, some or all of the particles of a compound described herein are microencapsulated. In some embodiments, the particles of the compound described herein are not microencapsulated and are uncoated.

An ASBT inhibitor may be used in the preparation of medicaments for the prophylactic and/or therapeutic treatment of cholestasis or a cholestatic liver disease. A method for treating any of the diseases or conditions described herein in an individual in need of such treatment, may involve administration of pharmaceutical compositions containing at least one ASBT inhibitor described herein, or a pharmaceutically acceptable salt, pharmaceutically acceptable N-oxide, pharmaceutically active metabolite, pharmaceutically acceptable prodrug, or pharmaceutically acceptable solvate thereof, in therapeutically effective amounts to said individual.

Classes of Pediatric Cholestatic Liver Disease

In one aspect of the present disclosure, the compositions and dosage forms comprising ASBTIs as described herein are suitable for treating or ameliorating pediatric cholestatic liver diseases. In some embodiments, the compositions and dosage forms comprising ASBTIs as described herein are suitable for treating or ameliorating pruritus. In some embodiments, the compositions and dosage forms comprising ASBTIs as described herein are suitable for treating or ameliorating hypercholemia. In some embodiments, the compositions and dosage forms comprising ASBTIs as described herein are suitable for treating or ameliorating xanthoma.

In certain embodiments, the cholestatic liver disease is progressive familial intrahepatic cholestasis (PFIC), PFIC type 1, PFIC type 2, PFIC type 3, Alagille syndrome, Dubin-Johnson Syndrome, biliary atresia, post-Kasai biliary atresia, post-liver transplantation biliary atresia, post-liver transplantation cholestasis, post-liver transplantation associated liver disease, intestinal failure associated liver disease, bile acid mediated liver injury, pediatric primary sclerosing cholangitis, MRP2 deficiency syndrome, neonatal sclerosing cholangitis, a pediatric obstructive cholestasis, a pediatric non-obstructive cholestasis, a pediatric extrahepatic cholestasis, a pediatric intrahepatic cholestasis, a pediatric primary intrahepatic cholestasis, a pediatric secondary intrahepatic cholestasis, benign recurrent intrahepatic cholestasis (BRIC), BRIP type 1, BRIC type 2, BRIC type 3, total parenteral nutrition associated cholestasis, paraneoplastic cholestasis, Stauffer syndrome, drug-associated cholestasis, infection-associated cholestasis, or gallstone disease. In some embodiments, the cholestatic liver disease is a pediatric form of liver disease.

In certain embodiments, a cholestatic liver disease is characterized by one or more symptoms selected from jaundice, pruritis, cirrhosis, hypercholemia, neonatal respiratory distress syndrome, lung pneumonia, increased serum concentration of bile acids, increased hepatic concentration of bile acids, increased serum concentration of bilirubin, hepatocellular injury, liver scarring, liver failure, hepatomegaly, xanthomas, malabsorption, splenomegaly, diarrhea, pancreatitis, hepatocellular necrosis, giant cell formation, hepatocellular carcinoma, gastrointestinal bleeding, portal hypertension, hearing loss, fatigue, loss of appetite, anorexia, peculiar smell, dark urine, light stools, steatorrhea, failure to thrive, and/or renal failure.

In certain embodiments, methods of the present invention comprise non-systemic administration of a therapeutically effective amount of an ASBTI. In certain embodiments, the methods comprise contacting the gastrointestinal tract, including the distal ileum and/or the colon and/or the rectum, of an individual in need thereof with an ASBTI. In various embodiments, the methods of the present invention cause a reduction in intraenterocyte bile acids, or a reduction in damage to hepatocellular or intestinal architecture caused by cholestasis or a cholestatic liver disease.

In various embodiments the subject has a condition associated with, caused by or caused in part by a BSEP deficiency. In certain embodiments, the condition associated with, caused by or caused in part by the BSEP deficiency is neonatal hepatitis, primary biliary cirrhosis (PBC), primary sclerosing cholangitis (PSC), PFIC 2, benign recurrent intrahepatic cholestasis (BRIC), intrahepatic cholestasis of pregnancy (ICP), drug-induced cholestasis, oral-contraceptive-induced cholestasis, biliary atresia, or a combination thereof.

In various embodiments, methods of the present invention comprise delivering to ileum or colon of the individual a therapeutically effective amount of any ASBTI described herein.

As used herein, “cholestasis” means the disease or symptoms comprising impairment of bile formation and/or bile flow. As used herein, “cholestatic liver disease” means a liver disease associated with cholestasis. Cholestatic liver diseases are often associated with jaundice, fatigue, and pruritus. Biomarkers of cholestatic liver disease include elevated serum bile acid concentrations, elevated serum alkaline phosphatase (AP), elevated gamma-glutamyltranspeptidease, elevated conjugated hyperbilirubinemia, and elevated serum cholesterol.

Cholestatic liver disease can be sorted clinicopathologically between two principal categories of obstructive, often extrahepatic, cholestasis, and nonobstructive, or intrahepatic, cholestasis. In the former, cholestasis results when bile flow is mechanically blocked, as by gallstones or tumor, or as in extrahepatic biliary atresia.

The latter group who has nonobstructive intrahepatic cholestasis in turn fall into two principal subgroups. In the first subgroup, cholestasis results when processes of bile secretion and modification, or of synthesis of constituents of bile, are caught up secondarily in hepatocellular injury so severe that nonspecific impairment of many functions can be expected, including those subserving bile formation. In the second subgroup, no presumed cause of hepatocellular injury can be identified. Cholestasis in such patients appears to result when one of the steps in bile secretion or modification, or of synthesis of constituents of bile, is constitutively damages. Such cholestasis is considered primary.

Accordingly, provided herein are methods and compositions for stimulating epithelial proliferation and/or regeneration of intestinal lining and/or enhancement of the adaptive processes in the intestine in individuals with cholestasis and/or a cholestatic liver disease. In some of such embodiments, the methods comprise increasing bile acid concentrations and/or GLP-2 concentrations in the intestinal lumen.

Hypercholemia, and elevated levels of AP (alkaline phosphatase), LAP (leukocyte alkaline phosphatase), gamma GT (gamma-glutamyl transpeptidase), and 5′-nucleotidase are biochemical hallmarks of cholestasis and cholestatic liver disease. Accordingly, provided herein are methods and compositions for stimulating epithelial proliferation and/or regeneration of intestinal lining and/or enhancement of the adaptive processes in the intestine in individuals with hypercholemia, and elevated levels of AP (alkaline phosphatase), LAP (leukocyte alkaline phosphatase), gamma GT (gamma-glutamyl transpeptidase or GGT), and/or 5′-nucleotidase. In some of such embodiments, the methods comprise increasing bile acid concentrations in the intestinal lumen. Further provided herein, are methods and compositions for reducing hypercholemia, and elevated levels of AP (alkaline phosphatase), LAP (leukocyte alkaline phosphatase), gamma GT (gamma-glutamyl transpeptidase), and 5′-nucleotidase comprising reducing overall serum bile acid load by excreting bile acid in the feces.

Pruritus is often associated with pediatric cholestasis and pediatric cholestatic liver diseases. It has been suggested that pruritus results from bile salts acting on peripheral pain afferent nerves. The degree of pruritus varies with the individual (i.e., some individuals are more sensitive to elevated levels of bile acids/salts). Administration of agents that reduce serum bile acid concentrations has been shown to reduce pruritus in certain individuals. Accordingly, provided herein are methods and compositions for stimulating epithelial proliferation and/or regeneration of intestinal lining and/or enhancement of the adaptive processes in the intestine in individuals with pruritus. In some of such embodiments, the methods comprise increasing bile acid concentrations in the intestinal lumen. Further provided herein, are methods and compositions for treating pruritus comprising reducing overall serum bile acid load by excreting bile acid in the feces.

Another symptom of pediatric cholestasis and pediatric cholestatic liver disease is the increase in serum concentration of conjugated bilirubin. Elevated serum concentrations of conjugated bilirubin result in jaundice and dark urine. The magnitude of elevation is not diagnostically important as no relationship has been established between serum levels of conjugated bilirubin and the severity of cholestasis and cholestatic liver disease. Conjugated bilirubin concentration rarely exceeds 30 mg/dL. Accordingly, provided herein are methods and compositions for stimulating epithelial proliferation and/or regeneration of intestinal lining and/or enhancement of the adaptive processes in the intestine in individuals with elevated serum concentrations of conjugated bilirubin. In some of such embodiments, the methods comprise increasing bile acid concentrations in the intestinal lumen. Further provided herein, are methods and compositions for treating elevated serum concentrations of conjugated bilirubin comprising reducing overall serum bile acid load by excreting bile acid in the feces.

Increased serum concentration of nonconjugated bilirubin is also considered diagnostic of cholestasis and cholestatic liver disease. Portions of serum bilirubin and covalently bound to albumin (delta bilirubin or biliprotein). This fraction may account for a large proportion of total bilirubin in patients with cholestatic jaundice. The presence of large quantities of delta bilirubin indicates long-standing cholestasis. Delta bilirubin in cord blood or the blood of a newborn is indicative of pediatric cholestasis/cholestatic liver disease that antedates birth. Accordingly, provided herein are methods and compositions for stimulating epithelial proliferation and/or regeneration of intestinal lining and/or enhancement of the adaptive processes in the intestine in individuals with elevated serum concentrations of nonconjugated bilirubin or delta bilirubin. In some of such embodiments, the methods comprise increasing bile acid concentrations in the intestinal lumen. Further provided herein, are methods and compositions for treating elevated serum concentrations of nonconjugated bilirubin and delta bilirubin comprising reducing overall serum bile acid load by excreting bile acid in the feces.

Pediatric cholestasis and cholestatic liver disease results in hypercholemia. During metabolic cholestasis, the hepatocytes retains bile salts. Bile salts are regurgitated from the hepatocyte into the serum, which results in an increase in the concentration of bile salts in the peripheral circulation. Furthermore, the uptake of bile salts entering the liver in portal vein blood is inefficient, which results in spillage of bile salts into the peripheral circulation. Accordingly, provided herein are methods and compositions for stimulating epithelial proliferation and/or regeneration of intestinal lining and/or enhancement of the adaptive processes in the intestine in individuals with hypercholemia. In some of such embodiments, the methods comprise increasing bile acid concentrations in the intestinal lumen. Further provided herein, are methods and compositions for treating hypercholemia comprising reducing overall serum bile acid load by excreting bile acid in the feces.

Hyperlipidemia is characteristic of some but not all cholestatic diseases. Serum cholesterol is elevated in cholestasis due to the decrease in circulating bile salts which contribute to the metabolism and degradation of cholesterol. Cholesterol retention is associated with an increase in membrane cholesterol content and a reduction in membrane fluidity and membrane function. Furthermore, as bile salts are the metabolic products of cholesterol, the reduction in cholesterol metabolism results in a decrease in bile acid/salt synthesis. Serum cholesterol observed in children with cholestasis ranges between about 1,000 mg/dL and about 4,000 mg/dL. Accordingly, provided herein are methods and compositions for stimulating epithelial proliferation and/or regeneration of intestinal lining and/or enhancement of the adaptive processes in the intestine in individuals with hyperlipidemia. In some of such embodiments, the methods comprise increasing bile acid concentrations in the intestinal lumen. Further provided herein, are methods and compositions for treating hyperlipidemia comprising reducing overall serum bile acid load by excreting bile acid in the feces.

In individuals with pediatric cholestasis and pediatric cholestatic liver diseases, xanthomas develop from the deposition of excess circulating cholesterol into the dermis. The development of xanthomas is more characteristic of obstructive cholestasis than of hepatocellular cholestasis. Planar xanthomas first occur around the eyes and then in the creases of the palms and soles, followed by the neck. Tuberous xanthomas are associated with chronic and long-term cholestasis. Accordingly, provided herein are methods and compositions for stimulating epithelial proliferation and/or regeneration of intestinal lining and/or enhancement of the adaptive processes in the intestine in individuals with xanthomas. In some of such embodiments, the methods comprise increasing bile acid concentrations in the intestinal lumen. Further provided herein, are methods and compositions for treating xanthomas comprising reducing overall serum bile acid load by excreting bile acid in the feces.

In children with chronic cholestasis, one of the major consequences of pediatric cholestasis and pediatric cholestatic liver disease is failure to thrive. Failure to thrive is a consequence of reduced delivery of bile salts to the intestine, which contributes to inefficient digestion and absorption of fats, and reduced uptake of vitamins (vitamins E, D, K, and A are all malabsorbed in cholestasis). Furthermore, the delivery of fat into the colon can result in colonic secretion and diarrhea. Treatment of failure to thrive involves dietary substitution and supplementation with long-chain triglycerides, medium-chain triglycerides, and vitamins. Accordingly, provided herein are methods and compositions for stimulating epithelial proliferation and/or regeneration of intestinal lining and/or enhancement of the adaptive processes in the intestine in individuals (e.g., children) with failure to thrive. In some of such embodiments, the methods comprise increasing bile acid concentrations in the intestinal lumen. Further provided herein, are methods and compositions for treating failure to thrive comprising reducing overall serum bile acid load by excreting bile acid in the feces.

In children with chronic cholestasis, an additional consequence of pediatric cholestasis and pediatric cholestatic liver disease is a reduction in growth relative to children not having pediatric cholestasis or pediatric cholestatic liver disease. Accordingly, provided herein are methods and compositions for stimulating epithelial proliferation and/or regeneration of intestinal lining and/or enhancement of the adaptive processes in the intestine in individuals (e.g., children) with reduced growth. In some of such embodiments, the methods comprise increasing bile acid concentrations in the intestinal lumen. Further provided herein, are methods and compositions for treating reduced growth comprising reducing overall serum bile acid load by excreting bile acid in the feces.

Progressive Familial Intrahepatic Cholestasis (PFIC)

PFIC is a rare genetic disorder that causes progressive liver disease typically leading to liver failure. In people with PFIC, liver cells are less able to secrete bile. The resulting buildup of bile causes liver disease in affected individuals. Signs and symptoms of PFIC typically begin in infancy. Patients experience severe itching, jaundice, failure to grow at the expected rate (failure to thrive), and an increasing inability of the liver to function (liver failure). The disease is estimated to affect one in every 50,000 to 100,000 births in the United States and Europe. Six types of PFIC have been genetically identified, all of which are similarly characterized by impaired bile flow and progressive liver disease.

### PFIC 1

PFIC 1 (also known as, Byler disease or FIC1 deficiency) is associated with mutations in the ATP8B1 gene (also designated as FIC1). This gene, which encodes a P-type ATPase, is located on human chromosome 18 and is also mutated in the milder phenotype, benign recurrent intrahepatic cholestasis type 1 (BRIO) and in Greenland familial cholestasis. FIC1 protein is located on the canalicular membrane of the hepatocyte but within the liver it is mainly expressed in cholangiocytes. P-type ATPase appears to be an aminophospholipid transporter responsible for maintaining the enrichment of phosphatidylserine and phophatidylethanolamme on the inner leaflet of the plasma membrane in comparison of the outer leaflet. The asymmetric distribution of lipids in the membrane bilayer plays a protective role against high bile salt concentrations in the canalicular lumen. The abnormal protein function may indirectly disturb the biliary secretion of bile acids. The anomalous secretion of bile acids/salts leads to hepatocyte bile acid overload.

PFIC 1 typically presents in infants (e.g., age 6-18 months). The infants may show signs of pruritus, jaundice, abdominal distension, diarrhea, malnutrition, and shortened stature. Biochemically, individuals with PFIC 1 have elevated serum transaminases, elevated bilirubin, elevated serum bile acid levels, and low levels of gammaGT. The individual may also have liver fibrosis. Individuals with PFIC 1 typically do not have bile duct proliferation. Most individuals with PFIC 1 will develop end-stage liver disease by 10 years of age. No medical treatments have proven beneficial for the long-term treatment of PFIC 1. In order to reduce extrahepatic symptoms (e.g., malnutrition and failure to thrive), children are often administered medium chain triglycerides and fat-soluble vitamins. Ursodiol has not been demonstrated as effective in individuals with PFIC 1.

### PFIC 2

PFIC 2 (also known as, Byler Syndrome, BSEP deficiency) is associated with mutations in the ABCB11 gene (also designated BSEP). The ABCB11 gene encodes the ATP-dependent canalicular bile salt export pump (BSEP) of human liver and is located on human chromosome 2. BSEP protein, expressed at the hepatocyte canalicular membrane, is the major exporter of primary bile acids/salts against extreme concentration gradients. Mutations in this protein are responsible for the decreased biliary bile salt secretion described in affected patients, leading to decreased bile flow and accumulation of bile salts inside the hepatocyte with ongoing severe hepatocellular damage.

PFIC 2 typically presents in infants (e.g., age 6-18 months). The infants may show signs of pruritus. Biochemically, individuals with PFIC 2 have elevated serum transaminases, elevated bilirubin, elevated serum bile acid levels, and low levels of gammaGT. The individual may also have portal inflammation and giant cell hepatitis. Further, individuals often develop hepatocellular carcinoma. No medical treatments have proven beneficial for the long-term treatment of PFIC 2. In order to reduce extrahepatic symptoms (e.g., malnutrition and failure to thrive), children are often administered medium chain triglycerides and fat-soluble vitamins. The PFIC 2 patient population accounts for approximately 60% of the PFIC population.

### PFIC 3

PFIC 3 (also known as MDR3 deficiency) is caused by a genetic defect in the ABCB4 gene (also designated MDR3) located on chromosome 7. Class III Multidrug Resistance (MDR3) P-glycoprotein (P-gp), is a phospholipid translocator involved in biliary phospholipid (phosphatidylcholine) excretion in the canalicular membrane of the hepatocyte. PFIC 3 results from the toxicity of bile in which detergent bile salts are not inactivated by phospholipids, leading to bile canaliculi and biliary epithelium injuries.

PFIC 3 also presents in early childhood. As opposed to PFIC 1 and PFIC 2, individuals have elevated gammaGT levels. Individuals also have portal inflammation, fibrosis, cirrhosis, and massive bile duct proliferation. Individuals may also develop intrahepatic gallstone disease. Ursodiol has been effective in treating or ameliorating PFIC 3.

Benign Recurrent Intrahepatic Cholestasis (BRIC)

### BRIC 1

BRIC1 is caused by a genetic defect of the FIC1 protein in the canalicular membrane of hepatocytes. BRIC1 is typically associated with normal serum cholesterol and γ-glutamyltranspeptidase levels, but elevated serum bile salts. Residual FIC1 expression and function is associated with BRIC1. Despite recurrent attacks of cholestasis or cholestatic liver disease, there is no progression to chronic liver disease in a majority of patients. During the attacks, the patients are severely jaundiced and have pruritus, steatorrhea, and weight loss. Some patients also have renal stones, pancreatitis, and diabetes.

### BRIC 2

BRIC2 is caused by mutations in ABCB11, leading to defective BSEP expression and/or function in the canalicular membrane of hepatocytes.

### BRIC 3

BRIC3 is related to the defective expression and/or function of MDR3 in the canalicular membrane of hepatocytes. Patients with MDR3 deficiency usually display elevated serum γ-glutamyltranspeptidase levels in the presence of normal or slightly elevated bile acid levels.

Dubin-Johnson Syndrome (DJS)

DJS is characterized by conjugated hyperbilirubinemia due to inherited dysfunction of MRP2. Hepatic function is preserved in affected patients. Several different mutations have been associated with this condition, resulting either in the complete absence of immunohistochemically detectable MRP2 in affected patients or impaired protein maturation and sorting.

Acquired Cholestatic Disease

### Pediatric Primary Sclerosing Cholangitis (PSC)

Pediatric PSC is a chronic inflammatory hepatic disorder slowly progressing to end stage liver failure in most of the affected patients. In pediatric PSC inflammation, fibrosis and obstruction of large and medium sized intra- and extrahepatic ductuli is predominant.

### Gallstone Disease

Gallstone disease is one of the most common and costly of all digestive diseases with a prevalence of up to 17% in Caucasian women. Cholesterol containing gallstones are the major form of gallstones and supersaturation of bile with cholesterol is therefore a prerequisite for gallstone formation. ABCB4 mutations may be involved in the pathogenesis of cholesterol gallstone disease.

### Drug Induced Cholestasis

Inhibition of BSEP function by drugs is an important mechanism of drug-induced cholestasis, leading to the hepatic accumulation of bile salts and subsequent liver cell damage. Several drugs have been implicated in BSEP inhibition. Most of these drugs, such as rifampicin, cyclosporine, glibenclamide, or troglitazone directly cis-inhibit ATP-dependent taurocholate transport in a competitive manner, while estrogen and progesterone metabolites indirectly trans-inhibits BSEP after secretion into the bile canaliculus by Mrp2. Alternatively, drug-mediated stimulation of MRP2 can promote cholestasis or cholestatic liver disease by changing bile composition.

Total Parenteral Nutrition Associated Cholestasis

TPNAC is one of the most serious clinical scenarios where cholestasis or cholestatic liver disease occurs rapidly and is highly linked with early death. Infants, who are usually premature and who have had gut resections are dependent upon TPN for growth and frequently develop cholestasis or cholestatic liver disease that rapidly progresses to fibrosis, cirrhosis, and portal hypertension, usually before 6 months of life. The degree of cholestasis or cholestatic liver disease and chance of survival in these infants have been linked to the number of septic episodes, likely initiated by recurrent bacterial translocation across their gut mucosa. Although there are also cholestatic effects from the intravenous formulation in these infants, septic mediators likely contribute the most to altered hepatic function.

Alagille Syndrome

Alagille syndrome is a genetic disorder that affects the liver and other organs. It often presents during infancy (e.g., age 6-18 months) through early childhood (e.g., age 3-5 years) and may stabilize after the age of 10. Symptoms may include chronic progressive cholestasis, ductopenia, jaundice, pruritus, xanthomas, congenital heart problems, paucity of intrahepatic bile ducts, poor linear growth, hormone resistance, posterior embryotoxon, Axenfeld anomaly, retinitis pigmentosa, pupillary abnormalities, cardiac murmur, atrial septal defect, ventricular septal defect, patent ductus arteriosus, and Tetralogy of Fallot. Individuals diagnosed with Alagille syndrome have been treated with ursodiol, hydroxyzine, cholestyramine, rifampicin, and phenobarbitol. Due to a reduced ability to absorb fat-soluble vitamins, individuals with Alagille Syndrome are further administered high dose multivitamins.

Biliary Atresia

Biliary atresia is a life-threatening condition in infants in which the bile ducts inside or outside the liver do not have normal openings. With biliary atresia, bile becomes trapped, builds up, and damages the liver. The damage leads to scarring, loss of liver tissue, and cirrhosis. Without treatment, the liver eventually fails, and the infant needs a liver transplant to stay alive. The two types of biliary atresia are fetal and perinatal. Fetal biliary atresia appears while the baby is in the womb. Perinatal biliary atresia is much more common and does not become evident until 2 to 4 weeks after birth.

### Post-Kasai Biliary Atresia

Biliary atresia is treated with surgery called the Kasai procedure or a liver transplant. The Kasai procedure is usually the first treatment for biliary atresia. During a Kasai procedure, the pediatric surgeon removes the infant's damaged bile ducts and brings up a loop of intestine to replace them. While the Kasai procedure can restore bile flow and correct many problems caused by biliary atresia, the surgery doesn't cure biliary atresia. If the Kasai procedure is not successful, infants usually need a liver transplant within 1 to 2 years. Even after a successful surgery, most infants with biliary atresia slowly develop cirrhosis over the years and require a liver transplant by adulthood. Possible complications after the Kasai procedure include ascites, bacterial cholangitis, portal hypertension, and pruritus.

### Post Liver Transplantation Biliary Atresia

If the atresia is complete, liver transplantation is the only option. Although liver transplantation is generally successful at treating biliary atresia, liver transplantation may have complications such as organ rejection. Also, a donor liver may not become available. Further, in some patients, liver transplantation may not be successful at curing biliary atresia.

Xanthoma

Xanthoma is a skin condition associated with cholestatic liver diseases, in which certain fats build up under the surface of the skin. Cholestasis results in several disturbances of lipid metabolism resulting in formation of an abnormal lipid particle in the blood called lipoprotein X. Lipoprotein X is formed by regurgitation of bile lipids into the blood from the liver and does not bind to the LDL receptor to deliver cholesterol to cells throughout the body as does normal LDL. Lipoprotein X increases liver cholesterol production by five-fold and blocks normal removal of lipoprotein particles from the blood by the liver.

All references cited anywhere within this specification are incorporated herein by reference in their entirety for all purposes.

While preferred embodiments of the present invention have been shown and described herein, it will be obvious to those skilled in the art that such embodiments are provided by way of example only.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### EXAMPLES

The following examples illustrate specific aspects of the instant description. The examples should not be construed as limiting, as the examples merely provide specific understanding and practice of the embodiments and their various aspects.

### Example 1: Formulations of Maralixibat

This example outlines various formulations of an ASBTI maralixibat according to embodiments of the present disclosure. The formulations are given in Table 1.

TABLE 1
Formulations of Maralixibat Oral Solutions According to an Embodiment of the Disclosure

| | | | mg/mL | | | | | |
|---|---|---|---|---|---|---|---|---|
| | | | 5 | 10 | 15 | 20 | 40 | 50 |
| Ingredient | Grade | Function | mg/mL | mg/mL | mg/mL | mg/mL | mg/mL | mg/mL |
| Maralixibat | In-House | Active | 5.00 | 10.00 | 15.00 | 20.00 | 40.00 | 50.00 |
| Chloride^{a} | | Pharmaceutical | | | | | | |
| | | Ingredient | | | | | | |
| Propylene | USP/Ph. Eur | Co-solvent | 360.00 | 360.00 | 360.00 | 360.00 | 360.00 | 360.00 |
| Glycol | | and | | | | | | |
| | | Preservative | | | | | | |
| Purified | USP/Ph. Eur/JP | Solvent | 649.00 | 644.00 | 639.00 | 634.00 | 614.00 | 604.00 |
| Water ^{b} | | | | | | | | |
| Disodium | USP/Ph. Eur | Antioxidant | 1.00 | 1.00 | 1.00 | 1.00 | 1.00 | 1.00 |
| EDTA | | | | | | | | |
| dihydrate | | | | | | | | |
| Sucralose | NF/Ph. Eur | Sweetener | 10.00 | 10.00 | 10.00 | 10.00 | 10.00 | 10.00 |
| Grape | Non- | Taste Masking | 5.00 | 5.00 | 5.00 | 5.00 | 5.00 | 5.00 |
| Flavor | compendial | Agent | | | | | | |

| | | | | | | |
|---|---|---|---|---|---|---|
| Total (mg) | 1030.00 | 1030.00 | 1030.00 | 1030.00 | 1030.00 | 1030.00 |
| Total (mL) ^{c} | 1.00 | 1.00 | 1.00 | 1.00 | 1.00 | 1.00 |

^{a}The quantity of maralixibat chloride is adjusted based on assay value. The weight of drug substance is based on maralixibat chloride. The dose can be converted into maralixibat free base by equation: amount of maralixibat free base = amount of maralixibat chloride × 0.95
^{b} Amount of purified water to be used is adjusted based on the assay of maralixibat chloride and in order maintain the weight of 1.00 mL of the solution.
^{c} The weight (mg) of the unit formula is converted to volume (mL) using the density of the respective solution

### Example 2: Antipruritic Medications for Treatment of ALGS

Patients with ALGS are typically treated with UDCA and rifampicin as well as other off label agents to control or reduce pruritus symptoms. These medications are usually only partially or temporarily effective in reducing the pruritus.

Eligibility for the maralixibat studies required moderate to severe pruritus, as measured by a score of 2 or greater on the ItchRO(Obs) instrument, irrespective of antipruritic background therapy. In Study LUM001-304, participants were not allowed to make any changes to their antipruritic therapy up to Week 22. In Studies LUM001-301 and LUM001-302, no changes to the antipruritic comedications were allowed throughout the period of the primary analysis up to Week 13. Therefore, across all studies, participants had to be on stable antipruritic medication doses (except for weight-based dose adjustments) throughout the randomized controlled periods.

Participants in LUM001-301 received either 70 μg/kg/day, 140 μg/kg/day, or 280 μg/kg/day of maralixibat (as maralixibat chloride). Participants in LUM001-302 received either 140 μg/kg/day or 280 μg/kg/day of maralixibat (as maralixibat chloride). The long-term LUM001-303 dosing was 280 μg/kg QD and 280 μg/kg BID. The long term LUM001-305 dosing was at 280 μg/kg/day.

After these stable-dosing periods in the long-term extension studies, changes in antipruritic medications were allowed. Weight-based dose adjustments in antipruritic medications were anticipated over the course of a 5-year study.

Table 2 shows the percentage of participants with one or more concomitant antipruritic medications at baseline.

TABLE 2
Prior Concomitant Antipruritic Medication at Baseline

| Number of prior | | | | | | |
|---|---|---|---|---|---|---|
| medications | LUM001-304 | | LUM001-303 | | LUM001-305 | |
| No Medications | 3 | (9.7%) | 0 | | 4 | (11.8%) |
| 1 Medication | 6 | (19.4%) | 1 | (5.3%) | 8 | (23.5%) |
| 2 Medications | 13 | (41.9%) | 5 | (26.3%) | 21 | (61.8%) |
| At Least 3 | 9 | (29.0%) | 13 | (68.4%) | 1 | (2.9%) |
| Medications | | | | | | |

After Week 22 of Study LUM001-304, of the 29 participants during the long-term extension, 10 participants experienced a decrease in concomitant antipruritic medication. Of these 10 participants, 3 participants stopped UDCA; 3 participants stopped rifampicin and UDCA; 1 participant stopped rifampicin and reduced UDCA; 3 participants stopped rifampicin. There was a change in medication for 2 participants who stopped rifampicin combined with an increase in UDCA. Additionally, 3 participants increased doses of UDCA; 1 participant stopped UDCA and started rifampicin; and 1 participant increased rifampicin. The remaining participants had no or minimal change in concomitant antipruritic medication.

In the Stable-dosing Period of Study LUM001-303, of the 19 participants during the long-term extension, 2 participants experienced a decrease in concomitant antipruritic medication: 1 participant stopped both UDCA and rifampicin; and 1 participant stopped rifampicin and reduced UDCA. Five participants experienced an increase in concomitant antipruritic medication: 1 participant increased doses of UDCA and rifampicin; 1 participant started rifampicin; 1 participant increased UDCA; and 2 participants increased doses of rifampicin with no change in UDCA. The remaining 12 participants had no or minimal change in concomitant antipruritic medication.

In the Stable-dosing Period of Study LUM001-305, of the 34 participants during the long term extension, 10 participants experienced a decrease in concomitant antipruritic medication. Of these 10 participants, 1 participant stopped UDCA; 1 participant stopped UDCA and rifampicin; 2 participants stopped rifampicin; 1 participant reduced UDCA and continued rifampicin; 1 participant reduced rifampicin and had a small increase in UDCA; 1 participant stopped UDCA and continued rifampicin; 1 participant stopped rifampicin and continued UDCA; 2 participants stopped rifampicin and increased UDCA. Seven participants experienced an increase in concomitant antipruritic medication: 2 participants increased doses of UDCA and rifampicin; 1 participant started rifampicin; 2 participants increased doses of rifampicin; and 2 participants had increases in UDCA. The remaining 17 participants had no or minimal change in concomitant antipruritic medication.

Almost all participants entered the ALGS studies with 1 to 3 antipruritic medications and still met entry criteria of moderate to severe pruritus. Overall, pruritus scores consistently improved during treatment with maralixibat over the long-term follow-up. In Study LUM001-304, the maralixibat 400 μg/kg dose demonstrated the greatest pruritus reduction and the largest proportion of participants reducing concomitant antipruritic medication. Supporting ALGS studies at lower doses showed a similar effect, albeit to a lesser degree.

These studies demonstrate that many patients receiving maralixibat in combination with UDCA and/or rifampicin were able to decrease the amount of UDCA and/or rifampicin. This indicates that reduced dosing of each medication was achieved through the combination treatment as compared to a monotherapy with either UDCA or rifampicin.

### Example 3: Antipruritic Medications for Treatment of PFIC

Patients with PFIC are typically treated with UDCA and rifampicin as well as other off-label agents to control or reduce pruritus symptoms. These medications are usually only partially or temporarily effective in reducing the pruritus.

In an open label study to evaluate efficacy and long term safety of maralixibat (LUM001) in the treatment of Cholestatic Liver Disease in patients with Progressive Familial Intrahepatic Cholestasis (PFIC) (Study LUM001-501), participants were not allowed to make any changes to their antipruritic therapy during the 13-week treatment period. No new medications used to treat pruritus were to be added during the 13-week treatment period. Therefore, participants had to be on stable antipruritic medication doses (except for weight-based dose adjustments) during the 13-week treatment period.

In the long-term exposure period, changes in antipruritic medications were allowed.

Table 3 shows the percentage of participants with one or more concomitant antipruritic medications at baseline reported as part of the PFIC disease history.

TABLE 3
Prior Concomitant Antipruritic Medication
at Study LUM001-501 Baseline

| Number of Prior | PFIC1 | PFIC2 | | Total | |
|---|---|---|---|---|---|
| Medications | (N = 8) | (N = 25) | | (N = 31) | |
| No Medications | 1 (12.5%) | 4 | (16.0%) | 5 | (15.2%) |
| 1 Medication | 1 (12.5%) | 1 | (4.0%) | 2 | (6.1%) |
| 2 Medications | 1 (12.5%) | 4 | (16.0%) | 5 | (15.2%) |
| At Least 3 | 5 (62.5%) | 16 | (64.0%) | 21 | (63.6%) |
| Medications | | | | | |

A post-hoc analysis of prior and concomitant antipruritic medications data from Study LUM001-501 in the maralixibat population (N=33) showed that 26 participants (83.9%) were administered antipruritic medications at baseline.

During the study, 5 participants had an increase in dose of antipruritic medication: 3 participants increased rifampicin (ItchRO[Obs]=1-3), 1 participant increased UDCA and rifampicin (ItchRO[Obs]=1), and 1 participant increased UDCA (ItchRO[Obs]=2-4).

During the study, 5 participants had modifications (increase, decrease, or discontinuation) in antipruritic medications: 3 participants stopped rifampicin and increased UDCA (ItchRO[Obs]=0-2), 1 participant increased rifampicin and reduced UDCA (ItchRO[Obs]=3-4), and 1 participant reduced rifampicin and increased UDCA (ItchRO[Obs]=3-4).

During the study, 13 participants had a decrease in dose or discontinuation of antipruritic medications: 3 participants stopped rifampicin (ItchRO[Obs]=0-2), 4 participants stopped UDCA (ItchRO[Obs]=0-3), 2 participants stopped UDCA and rifampicin (ItchRO[Obs]=0-3), 1 participant reduced rifampicin (ItchRO[Obs]=1), 2 participants reduced UDCA (ItchRO[Obs]=1), and 1 participant stopped rifampicin and reduced UDCA (ItchRO[Obs]=2).

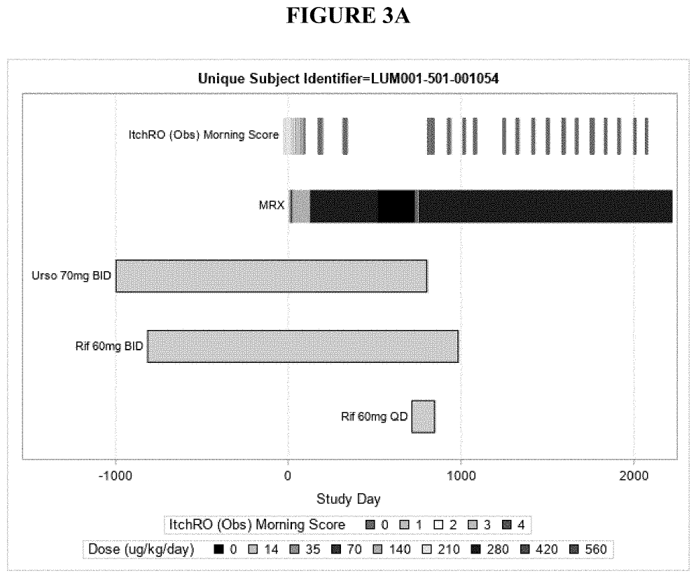

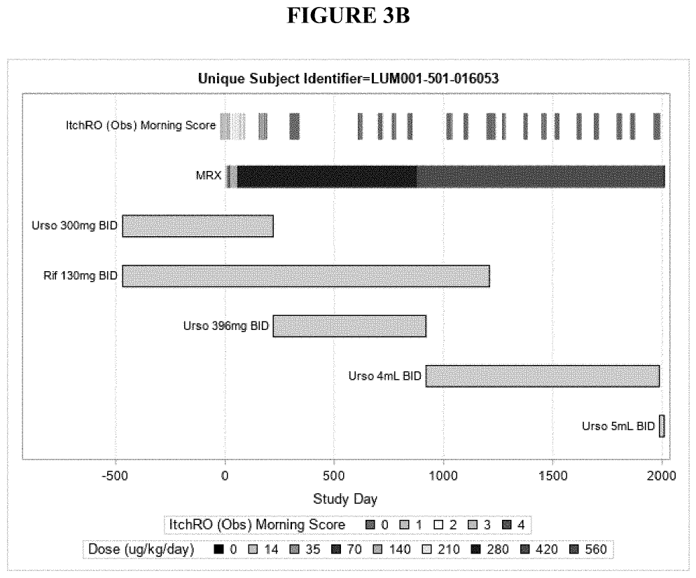

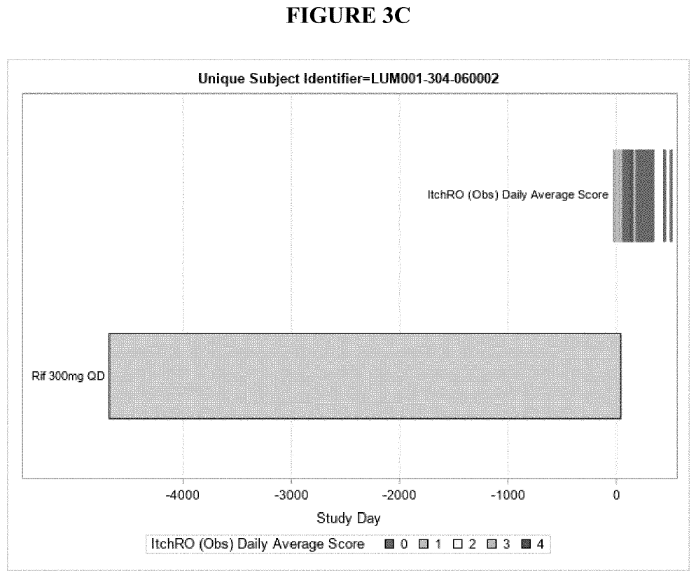

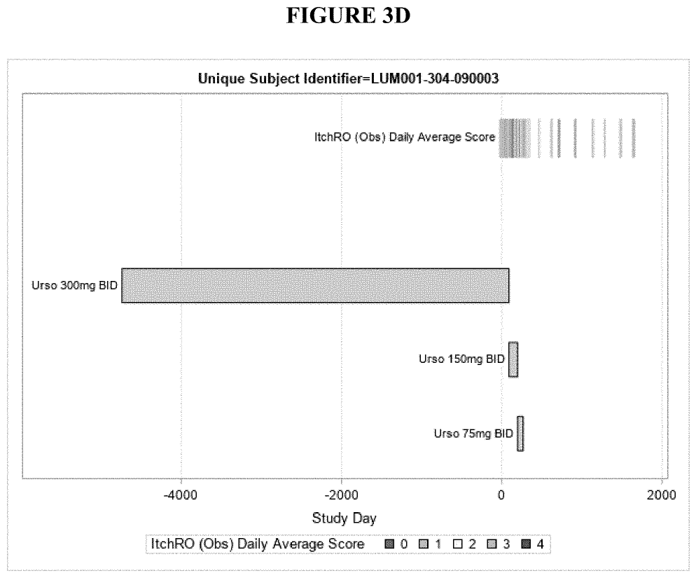

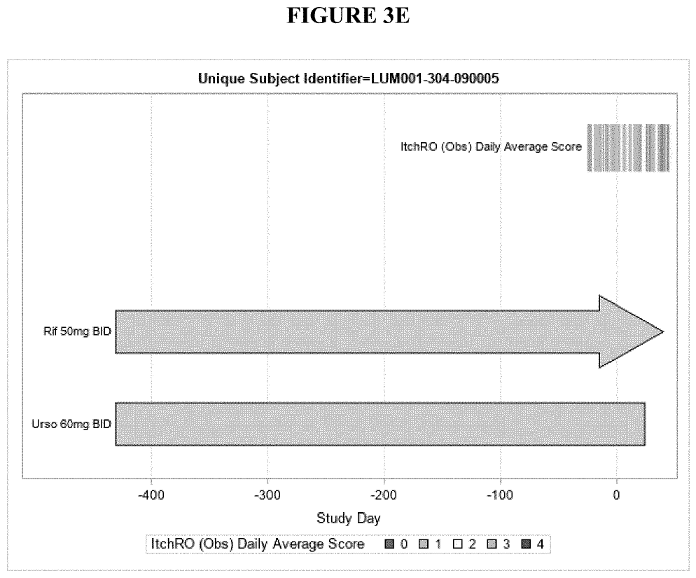

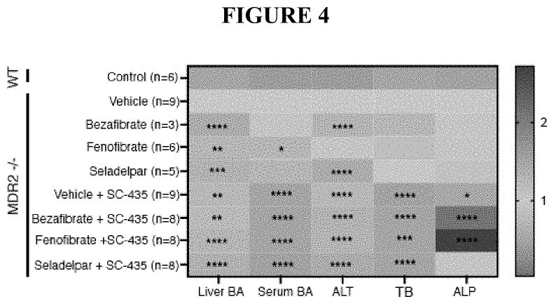

FIGS. 3A-3E are plots of ItchRO and doses of maralixibat and selected antipruritic medications for 5 exemplary PFIC patients enrolled in the LUM001-501 Study. These figures demonstrate that the patients were able to not only reduce the dosing, but entirely discontinue one or more antipruritic medications and still maintain control of pruritus while on maralixibat. Specifically, FIG. 3A shows the patient discontinued both Rifampicin and UDCA while maintaining excellent pruritus control. FIGS. 3B and 3C show the patients discontinued Rifampicin while maintaining excellent pruritus control. FIGS. 3D and 3E show the patients discontinued UDCA while maintaining pruritus control.

Overall, a greater number of participants reduced and/or discontinued antipruritic medications coupled with general improvements in pruritus as demonstrated by the reduced ItchRO(Obs) scores over a sustained period of follow-up. This indicates that reduced dosing of medication was achieved through the combination treatment.

### Example 4: Development of Maralixibat Oral Solution

Since pediatric patients are the target patient population for the proposed cholestatic disease, an oral solution formulation was selected due to its flexibility for dose adjustment based on patient's body weight and the preference for this type of formulation in young children. As maralixibat chloride is highly water soluble, with a water solubility of >100 mg/mL, it is a good candidate for solution formulation.

Therefore, oral solutions with maralixibat chloride (fixed dosage volume “FDV” and fixed drug substance concentration “FDSC” formulations) were developed to support the final commercial drug product formulation. The development of these oral solution formulation is described below.

### Example 4: Fixed Dose Volume (FDV) Formulation

### Solubility

For early pediatric clinical studies, the desired maralixibat solution concentration was in the range of 0.02 to 20 mg/mL (concentration based on maralixibat chloride). Initial formulation studies were conducted to determine the solubility and stability of MRX drug substance in three liquid oral dosing vehicles: water, Pedialyte® (an oral rehydration preparation), and Ora-Sweet® SF (a sugar-free, alcohol-free syrup vehicle for oral preparations). Using these three vehicles, samples were produced with the MRX drug substance at three different concentrations (0.02 mg/mL, 2.0 mg/mL, and 4.0 mg/mL concentrations based on maralixibat chloride). A vortex mixer was used to disperse the drug within the liquid vehicle; drug dissolution status was then visually checked and recorded as in Table 4.

TABLE 4
Solubility of Maralixibat (MRX) Drug Substance in Water
and Commercially Available Vehicles, Visual Observation

| | Concentration | |
|---|---|---|
| Vehicle | (mg/mL) | Solubility Observations |
| Water | 0.02 | Dissolved Instantly |
| | 2.0 | Dispersed, initially cloudy; |
| | | after 15 min in 40° C. water bath |
| | | completely dissolved. |
| | 4.0 | Dispersed, initially cloudy; |
| | | after 25 min in 40° C. water bath |
| | | completely dissolved. |
| Pedialyte ® | 0.02 | Dispersed, slightly cloudy; |
| | | after 60 min in 40° C. water bath no |
| | | change; sitting overnight no change. |
| | 2.0 | Dispersed, slightly cloudy; |
| | | after 60 min in 40° C. water bath no |
| | | change; sitting overnight MRX |
| | | settled out. |
| | 4.0 | Dispersed, slightly cloudy; |
| | | after 60 min in 40° C. water bath no |
| | | change; sitting overnight MRX |
| | | settled out. |
| Ora-Sweet ® | 0.02 | Did not disperse; after 60 min in |
| SF | | 40° C. water bath drug began to |
| | | disperse; sitting overnight no change. |
| | 2.0 | Did not disperse; after 60 min in |
| | | 40° C. water bath drug began to |
| | | disperse; sitting overnight no change. |
| | 4.0 | Did not disperse; after 60 min in |
| | | 40° C. water bath drug began to |
| | | disperse; sitting overnight no change. |

As presented in Table 4, results showed that among the three vehicles, water was the only solvent that provided acceptable solubility for the maralixibat drug substance. To utilize the taste masking property of Ora-Sweet®, the vehicle was mixed with a portion of the 4.0 mg/mL MRX water solution. Upon addition, gel formation and phase separation of the materials were observed. No improvement was observed on the appearance of the formulation even when the mixture was heated in a water bath.

### Solvent System

It was determined that the commercially available oral vehicles alone would not provide adequate solubility for the MRX drug substance at desired concentrations. To find an optimal vehicle for the MRX substance, different solvents and their combinations were explored. Polyethylene glycol (PEG) 300, propylene glycol, glycerin, water, and ethanol were assessed. The visual observations for the solubility of MRX drug substance at a concentration of 4 mg/mL with various solvents are provided in Table 5.

TABLE 5
Solubility of MRX Drug Substance in Different
Solvent Systems, 4 mg/mL, Visual Observation

| Solvent | Solubility Observation |
|---|---|
| PEG 300 | Dispersed, initially hazy; slowly |
| | dissolved; API particles remain |
| | after sitting overnight. |
| Propylene Glycol | Dispersed, initially hazy; dissolved |
| | within 4 hours and remained in |
| | solution overnight. |
| 1:1 Propylene | Dispersed; clear solution with minimal |
| Glycol:Water | undissolved particles within 5 minutes; |
| | complete dissolution after 20 minutes. |
| 1:1 PEG 300:Water | Dispersed, hazy; mostly clear solution |
| | with minimal undissolved particles after |
| | 20 minutes; complete dissolution in 75 minutes. |
| 1:3 Propylene | Dispersed, partly hazy, mostly clear |
| Glycol:Water | solution with minimal undissolved particles |
| | after 30 minutes; complete dissolution |
| | after 120 minutes. |
| 1:3 PEG 300:Water | Dispersed, mostly hazy; partly hazy after |
| | 60 minutes; minimal undissolved particles |
| | after 120 minutes; sitting overnight very |
| | little undissolved API particles remain. |
| Glycerin | Very viscous, took vigorous mixing to |
| | disperse, hazy; after 2 hours no change |
| | in dissolution. |
| 1:1 Glycerin:Propylene | Slightly viscous, took vigorous mixing |
| glycol | to disperse, moderately hazy; after 30 |
| | minutes slight dissolution; after 2 |
| | hours no change in dissolution. |
| 1:1 Glycerin:Water | Took moderate mixing to disperse, hazy; |
| | after 2 hours no change in dissolution. |
| 5:4:1 Propylene | Dispersed; complete dissolution within |
| glycol:Water:Ethanol | 5 minutes. |
| 2:7:1 Propylene | Dispersed; complete dissolution within |
| glycol:Water:Ethanol | 10 minutes. |
| 1:9 Propylene | Dispersed, hazy; after 90 minutes sill |
| glycol:Water | slightly hazy. |
| 1:8.9:0.1 Propylene | API clumped together; hazy dispersion; |
| glycol, | after 90 minutes slightly hazy with |
| Water:SLS | undissolved clumps of API. |
| 2.5:7.4:0.1 Propylene | Dispersed; complete dissolution within |
| glycol:Water:Ethanol | 8 minutes. |
| 2.5:7.4:0.1 Propylene | Dispersed, hazy; after 60 minutes mostly |
| glycol:Water:Citric Acid | dissolved with minimal undissolved API. |

Abbreviations: API = active pharmaceutical ingredient; SLS = Sodium Laurel Sulfate

Based on the results presented in Table 5, a combination of propylene glycol, water, and ethanol provided the most favorable option for the development of a solution formulation for maralixibat. However, since the formulation was intended for pediatric use, ethanol was removed from the solvent system. Sucralose and a flavoring agent (Grape Flavor F-9924 PFC) were added as sweetener and taste masking agent, respectively.

Five prototype solvent systems were explored for maralixibat drug substance as presented in Table 6.

TABLE 6
Prototypes of Solvent Systems for Maralixibat Oral Solution

| | Prototype | | | | |
|---|---|---|---|---|---|
| Components (% w/w) | 1 | 2 | 3 | 4 | 5 |
| Propylene Glycol | 25.00 | 25.00 | 25.00 | 25.00 | 25.00 |
| Water (deionized) | 74.50 | 74.00 | 73.50 | 73.00 | 73.50 |
| Ethanol | — | — | 1.00 | 1.00 | — |
| Sucralose | 0.50 | 0.50 | 0.50 | 0.50 | 0.75 |
| Grape Flavor | — | 0.50 | — | 0.50 | 0.75 |
| Total | 100.00 | 100.00 | 100.00 | 100.00 | 100.00 |

The prototype solvent systems were made by mixing the solvents to create a solvent mixture, followed by addition of sucralose and the grape flavoring under agitation. To produce the active solutions, an aliquot of the prototype solvent systems was added to a vial containing a certain amount of MRX drug substance (to achieve the desired dose) and mixed manually. A concentration of 4.0 mg/mL (maralixibat chloride) was targeted initially as the highest dose for development. Prototype 5 was selected as the diluent for further drug product formulation development.

### Diluent

A bulk diluent of Prototype 5 was prepared and used to produce two MRX oral solution formulations of 0.02 mg/mL and 4.0 mg/mL (concentrations expressed as maralixibat chloride). As suggested by the short-term stability results (Table 7 and Table 8, below), no significant changes in assay, pH, and impurity profiles were observed for both solutions stored under 2° C.-8° C. and 25° C./60% RH for up to 14 days. As clinical development progressed, a higher amount of the MRX drug substance was required to account for proposed changes in the dosing regimen. The content of the grape flavor was also reduced from 0.75 to 0.5% w/w in the formulation. The final composition of the diluent to be used with MRX drug substance for the MRX oral solution is presented in Table 9.

TABLE 7
Stability of MRX Oral Solution in Diluent^{a}

| | | Concentration | | | | | | | |
|---|---|---|---|---|---|---|---|---|---|
| | | 0.02 mg/mL^{b} | | | | 4.0 mg/m^{b} | | | |
| | | Storage Condition | | | | | | | |
| | | 2° C.-8° C. | | 25° C./60% RH | | 2° C.-8° C. | | 25° C./60% RH | |
| | | Time Point | | | | | | | |
| | | T = 0 | 14 Days | T = 0 | 14 Days | T = 0 | 14 Days | T = 0 | 14 Days |
| | | Results | | | | | | | |
| Assay (%) | | 105^{c} | 91 | 104 | 86 | 105 | 100 | 98 | 96 |
| Impurities | RRT 0.91 | ND | ND | ND | ND | 0.09 | 0.07 | 0.09 | 0.19 |
| | RRT1.21 | 0.50 | 0.26 | 0.50 | 0.60 | 0.38 | 0.40 | 0.38 | 0.35 |
| | RRT1.31 | ND | ND | ND | ND | 0.16 | 0.14 | 0.16 | 0.13 |
| | RRT1.47 | ND | ND | ND | ND | 0.31 | 0.28 | 0.31 | 0.31 |
| | RRT1.81 | ND | ND | ND | ND | 0.13 | 0.07 | 0.13 | 0.11 |
| | Total | 0.50 | 0.26 | 0.50 | 0.60 | 0.94 | 0.96 | 0.94 | 1.09 |
| | Impurities | | | | | | | | |

Abbreviations: ND = not detected; RRT = relative retention time
^{a}Diluent Batch 2012-041-54
^{b}Concentration based on maralixibat chloride.
^{c}Data from two retest sample

TABLE 8
MRX Oral Solution in Diluent, pH Value

| Time | 0.02 mg/mL^{a} | | 4.0 mg/mL^{a} | |
|---|---|---|---|---|
| Point | 2° C.-8° C. | 25° C./60% RH | 2° C.-8° C. | 25° C./60% RH |
| T = 0 | 3.66 | | 3.68 | |
| T = 7 days | 3.85 | 3.85 | 3.86 | 3.81 |
| T = 14 days | 3.87 | 3.87 | 3.85 | 3.88 |

^{a}Diluent Batch 2012-041-54

TABLE 9
Diluent Formulation

| Ingredients | Quality Standard | % w/w |
|---|---|---|
| Propylene Glycol | USP, Ph. Eur. | 25.00 |
| Purified Water | USP, Ph. Eur. | 73.50 |
| Sucralose | NF, Ph. Eur. | 0.75 |
| Grape Flavor | In-House | 0.75 |

| | |
|---|---|
| Total | 100 |

### Stability

Additional studies were performed to evaluate maralixibat solution at a wider concentration range. Using the diluent described in Table 9, MRX oral solution was prepared and assessed at concentrations ranging from 10 mg/mL to 50 mg/mL (concentrations based on maralixibat chloride). All solutions were clear after a few hours of mixing at room temperature. These prepared solutions are referred to as fixed dosing volumes (FDV) formulations.

Representative long-term stability of the diluent for MRX oral solution at 25° C./60% RH is presented in Table 10A, below.

TABLE 10A
Stability of the Diluent at 25° C./60% RH

| Quality | Acceptance | | | | | |
|---|---|---|---|---|---|---|
| Attribute(s) | Criteria | Initial | 1 month | 6 months | 12 months | 24 months |
| Appearance | Colorless to | Conform | Conform | Conform | Conform | Conform |
| | light | | | | | |
| | yellow | | | | | |
| | solution | | | | | |
| pH | Report Results | 4.04 | 4.08 | 4.01 | 4.09 | 4.05 |
| Propylene | 90-110% | 100% | 100% | NT | 102% | 103% |
| Glycol | theoretical | | | | | |
| Assay | amount | | | | | |
| Microbial | Total Viable | <10 CFU/g | NT | NT | <100 CFU/g | <100 CFU/g |
| Limits | Aerobic | | | | | |
| | Count: ≤10² CFU/g | | | | | |
| | Total Mold and | <10 CFU/g | NT | NT | <10 CFU/g | <10 CFU/g |
| | Yeast: ≤10¹ CFU/g | | | | | |
| | E. coli.: | Absent | NT | NT | Absent | Absent |
| | Absent/g | | | | | |

^{a} Abbreviations: CFU = colony forming unit; NT = not tested

Results from the stability study met the acceptance criteria applicable at the time of testing. Therefore, the results indicate that all quality attributes of the diluent for the MRX oral solution are stable up to 24 months when stored at 25° C./60 RH.

These FDV formulations were placed on stability for up to 24 months at 2° C.-8° C. and 25° C./60% RH. Table 10B provides the stability design for FDV formulations. Table 11 provides a summary of the solutions, container closure and stability storage conditions for the batches manufactured. Stability data met specifications applicable at the time of testing, and therefore, supported the use of the FDV formulation at concentrations up to 50 mg/mL (expressed as maralixibat chloride) for the clinical studies applicable at the time.

TABLE 10B
Stability Design for FDV Formulation

| MRX Oral | | | | |
|---|---|---|---|---|
| Solution | | Fill | | Study |
| Concentration | Container | Volume | Storage | Duration |
| (mg/mL)^{a} | Closure | (mL) | Condition | (month) |
| 10 | 20 mL glass | 10 | 2° C.-8° C. | 14 |
| | scintillation | | 25° C./60% RH | 14 |
| 20 | vials | 10 | 2° C.-8° C. | 24 |
| | with cap | | 25° C./60% RH | 12 |
| 35 | | 10 | 2° C.-8° C. | 24 |
| | | | 25° C./60% RH | 12 |
| 50 | | 10 | 2° C.-8° C. | 24 |
| | | | 25° C./60% RH | 12 |

^{a}Quantity is expressed as maralixibat chloride (salt form), which can be converted to maralixibat (free base) using a conversion factor of 0.95.

TABLE 11
Stability Summary for FDV Formulation (10 mg/mL) at 2° C.-8° C.

| Attribute | Specification | Initial | 6 months | 9.5 months | 14 months |
|---|---|---|---|---|---|
| Appearance | Colorless to | conform | conform | conform | conform |
| | slightly | | | | |
| | yellow | | | | |
| | solution | | | | |
| pH | Report Result | 3.62 | 4.12 | 4.58 | 4.23 |
| Assay (mg/mL) | Report Result | 9.8 | 9.9 | 9.6 | 9.9 |
| Single Largest | Report Results | RRT 0.90 = | RRT 0.90 = | RRT 0.90 = | RRT 0.90 = |
| Impurity^{a} | | 0.52 | 0.65 | 1.02 | 0.72 |
| Total % | Report Results | 0.88 | 1.67 | 1.44 | 0.72 |
| impurities^{b} | | | | | |

Abbreviations: RRT = relative retention time
^{a}Impurity peak reported at RRT 0.44 not included as it is diluent related.
^{b}Total % Impurities is the sum of peaks ≥ 0.05%.

TABLE 12
Stability Summary for FDV Formulation (10 mg/mL) at 25° C./60% RH

| | Acceptance | | | | |
|---|---|---|---|---|---|
| Attribute | Criteria | Initial | 6 months | 9.5 months | 14 months |
| Appearance | Colorless to | Conform | Conform | Conform | Conform |
| | slightly | | | | |
| | yellow | | | | |
| | solution | | | | |
| pH | Report Result | 3.62 | 4.44 | 4.82 | 4.33 |
| Assay (mg/mL) | Report Result | 9.8 | 10.3 | 10.6 | 12.2 |
| Single Largest | Report Result | RRT 0.90 = | RRT 0.90 = | RRT 0.90 = | RRT 0.90 = |
| Impurity^{a} | | 0.52 | 0.82 | 1.39 | 1.02 |
| Total % | Report Results | 0.88 | 1.78 | 1.93 | 1.38 |
| Impurities^{b} | | | | | |

Abbreviations: RRT = relative retention time
^{a}Impurity peak reported at RRT 0.44 not included as it is diluent related.
^{b}Total % Impurities is the sum of peaks ≥ 0.05%.

TABLE 13
Stability Summary for FDV Formulation (20 mg/mL) at 2° C.-8° C.

| | | Timepoint (month) | | | | | | | |
|---|---|---|---|---|---|---|---|---|---|
| Attribute | Specification | Initial | 1 | 3 | 6 | 9 | 12 | 18 | 24 |
| Appearance | Colorless to | conform | conform | conform | conform | conform | conform | conform | conform |
| | slightly | | | | | | | | |
| | yellow | | | | | | | | |
| | solution | | | | | | | | |
| pH | Report Result | 4.15 | 4.13 | 4.23 | 4.28 | 4.18 | 4.16 | 4.55 | 4.23 |
| Assay | 90.-110.0% | 94.8 | 94.7 | 92.9 | 94.5 | 97.5 | 97.7 | 99.3 | 98.5 |
| Single | Report Results | RRT 1.45 = | RRT 1.45 = | RRT 1.45 = | RRT 1.45 = | RRT 0.90 = | RRT 0.92 = | RRT 0.90 = | RRT 0.90 = |
| Largest | | 0.26 | 0.20 | 0.21 | 0.26 | 0.28 | 0.34 | 0.36 | 0.51 |
| Impurity | | | | | | | | | |
| Total % | Report Results | 0.42 | 0.40 | 0.40 | 0.40 | 0.68 | 0.62 | 0.71 | 0.74 |
| Impurities ^{a} | | | | | | | | | |

Microbial Limits Testing

| | | | | | | | | | |
|---|---|---|---|---|---|---|---|---|---|
| Total Viable | ≤1000 CFU/g | <10 CFU/g | NT | NT | NT | NT | <10 CFU/g | NT | <10 CFU/g |
| Aerobic Count | | | | | | | | | |
| Total Combined | ≤100 CFU/g | <10 CFU/g | NT | NT | NT | NT | <10 CFU/g | NT | <10 CFU/g |
| Yeasts and | | | | | | | | | |
| Molds | | | | | | | | | |
| E. Coli | Absent/1 g | Absent | NT | NT | NT | NT | Absent | NT | Absent |

Abbreviations: NT = not tested; RRT = relative retention time
^{a} Total % Impurities is the sum of peaks ≥ 0.05%.

TABLE 14
Stability Summary for FDV Formulation (20 mg/mL) at 25° C./60% RH

| | | Timepoint (month) | | | |
|---|---|---|---|---|---|
| Attribute | Specification | Initial | 1 | 3 | 6 |
| Appearance | Colorless to | conform | conform | conform | conform |
| | slightly | | | | |
| | yellow | | | | |
| | solution | | | | |
| pH | Report Result | 4.15 | 4.32 | 4.23 | 4.36 |
| Assay | 90.0-110.0% | 94.8 | 95.2 | 95.2 | 93.8 |
| Single Largest | Report Results | RRT 1.45 = | RRT 1.47 = | RRT 1.47 = | RRT 1.45 = |
| Impurity | | 0.26 | 0.20 | 0.21 | 0.25 |
| Total % | Report Results | 0.42 | 0.35 | 0.40 | 0.41 |
| Impurities^{a} | | | | | |

Microbial Limits Testing

| | | | | | |
|---|---|---|---|---|---|
| Total Viable | ≤1000 CFU/g | <10 CFU/g | NT | NT | NT |
| Aerobic Count | | | | | |
| Total Combined | ≤100 CFU/g | <10 CFU/g | NT | NT | NT |
| Yeasts and Molds | | | | | |
| E. Coli | Absent/1 g | Absent | NT | NT | NT |

Abbreviations: NT = not tested; RRT = relative retention time
^{a}Total % Impurities is the sum of peaks ≥ 0.05%.

TABLE 15
Stability Summary for FDV Formulation (35 mg/mL) at 2° C.-8° C.

| | | Timepoint (month) | | | | | | | |
|---|---|---|---|---|---|---|---|---|---|
| Attribute | Specification | Initial | 1 | 3 | 6 | 9 | 12 | 18 | 24 |
| Appearance | Colorless to | conform | conform | conform | conform | conform | conform | conform | conform |
| | slightly | | | | | | | | |
| | yellow | | | | | | | | |
| | solution | | | | | | | | |
| pH | Report Result | 4.21 | 4.37 | 4.19 | 4.16 | 4.82 | 4.30 | 4.27 | 4.29 |
| Assay | 90.0-110.0% | 93.2 | 92.9 | 91.4 | 92.9 | 94.2 | 94.7 | 95.5 | 95.1 |
| Single | Report Results | RRT 1.46 = | RRT 1.47 = | RRT 1.47 = | RRT 1.47 = | RRT 1.44 = | RRT 0.92 = | RRT 0.90 = | RRT 0.90 = |
| Largest | | 0.25 | 0.20 | 0.20 | 0.26 | 0.25 | 0.32 | 0.31 | 0.38 |
| Impurity | | | | | | | | | |
| Total % | Report Results | 0.41 | 0.36 | 0.33 | 0.42 | 0.63 | 0.67 | 0.65 | 0.67 |
| Impurities^{a} | | | | | | | | | |

Microbial Limits Testing

| | | | | | | | | | |
|---|---|---|---|---|---|---|---|---|---|
| Total Viable | ≤1000 CFU/g | <10 CFU/g | NT | NT | NT | NT | <10 CFU/g | NT | <10 CFU/g |
| Aerobic Count | | | | | | | | | |
| Total Combined | ≤100 CFU/g | <10 CFU/g | NT | NT | NT | NT | <10 CFU/g | NT | <10 CFU/g |
| Yeasts and | | | | | | | | | |
| Molds | | | | | | | | | |
| E. Coli | Absent/1 g | Absent | NT | NT | NT | NT | Absent | NT | Absent |

Abbreviations: NT = not tested; RRT = relative retention time
^{a}Total % Impurities is the sum of peaks ≥ 0.05%.

TABLE 16
Stability Summary for FDV Formulation (35 mg/mL) at 25° C./60% RH

| | | Timepoint (month) | | | |
|---|---|---|---|---|---|
| Attribute | Specification | Initial | 1 | 3 | 6 |
| Appearance | Colorless to | conform | conform | conform | conform |
| | slightly | | | | |
| | yellow | | | | |
| | solution | | | | |
| pH | Report Result | 4.21 | 4.37 | 4.33 | 4.25 |
| Assay | 90.0-110.0% | 93.2 | 93.1 | 91.9 | 93.4 |
| Single Largest | Report Results | RRT 1.46 = | RRT 1.47 = | RRT 1.47 = | RRT 1.47 = |
| Impurity | | 0.22 | 0.19 | 0.20 | 0.26 |
| Total % | Report Results | 0.41 | 0.41 | 0.39 | 0.49 |
| Impurities^{a} | | | | | |

Microbial Limits Testing

| | | | | | |
|---|---|---|---|---|---|
| Total Viable | ≤1000 CFU/g | <10 CFU/g | NT | NT | NT |
| Aerobic Count | | | | | |
| Total Combined | ≤100 CFU/g | <10 CFU/g | NT | NT | NT |
| Yeasts and Molds | | | | | |
| E. Coli | Absent/1 g | Absent | NT | NT | NT |

Abbreviations: NT = not tested; RRT = relative retention time
^{a}Total % Impurities is the sum of peaks ≥ 0.05%.

TABLE 17
Stability Summary for FDV Formulation (50 mg/mL) at 2° C.-8° C.

| | | Timepoint (month) | | | | | | | |
|---|---|---|---|---|---|---|---|---|---|
| Attribute | Specification | Initial | 1 | 3 | 6 | 9 | 12 | 18 | 24 |
| Appearance | Colorless to | conform | conform | conform | conform | conform | conform | conform | conform |
| | slightly | | | | | | | | |
| | yellow | | | | | | | | |
| | solution | | | | | | | | |
| pH | Report Result | 4.28 | 4.42 | 4.30 | 4.30 | 4.36 | 4.40 | 4.41 | 4.44 |
| Assay | 90.0-110.0% | 94.5 | 92.8 | 91.9 | 93.9 | 95.5 | 95.2 | 96.0 | 93.8 |
| Single | Report Results | RRT 1.46 = | RRT 1.47 = | RRT 1.47 = | RRT 1.47 = | RRT 1.44 = | RRT 0.92 = | RRT 0.90 = | RRT 0.90 = |
| Largest | | 0.27 | 0.20 | 0.20 | 0.24 | 0.28 | 0.26 | 0.30 | 0.36 |
| Impurity | | | | | | | | | |
| Total % | Report Results | 0.38 | 0.36 | 0.33 | 0.38 | 0.74 | 0.61 | 0.64 | 0.67 |
| Impurities^{a} | | | | | | | | | |

Microbial Limits Testing

| | | | | | | | | | |
|---|---|---|---|---|---|---|---|---|---|
| Total Viable | ≤1000 CFU/g | <10 CFU/g | NT | NT | NT | NT | <10 CFU/g | NT | <10 CFU/g |
| Aerobic Count | | | | | | | | | |
| Total Yeasts | ≤100 CFU/g | <10 CFU/g | NT | NT | NT | NT | <10 CFU/g | NT | <10 CFU/g |
| and Molds | | | | | | | | | |
| E. Coli | Absent/1 g | Absent | NT | NT | NT | NT | Absent | NT | Absent |

Abbreviations: NT = not tested; RRT = relative retention time
^{a}Total % Impurities is the sum of peaks ≥ 0.05%.

TABLE 18
Stability Summary for FDV Formulation (50 mg/mL) at 25° C./60% RH

| | | Timepoint (month) | | | |
|---|---|---|---|---|---|
| Attribute | Specification | Initial | 1 | 3 | 6 |
| Appearance | Colorless to | conform | conform | conform | conform |
| | slightly | | | | |
| | yellow | | | | |
| | solution | | | | |
| pH | Report Result | 4.28 | 4.45 | 4.39 | 4.38 |
| Assay | 90.0-110.0% | 94.5 | 92.3 | 92.2 | 93.4 |
| Single Largest | Report Results | RRT 1.46 = | RRT 1.47 = | RRT 1.47 = | RRT 1.47 = |
| Impurity | | 0.27 | 0.19 | 0.20 | 0.26 |
| Total % | Report Results | 0.38 | 0.30 | 0.39 | 0.45 |
| Impurities^{a} | | | | | |

Microbial Limits Testing

| | | | | | |
|---|---|---|---|---|---|
| Total Viable | ≤1000 CFU/g | <10 CFU/g | NT | NT | NT |
| Aerobic Count | | | | | |
| Total Combined | ≤100 CFU/g | <10 CFU/g | NT | NT | NT |
| Yeasts and Molds | | | | | |
| E. Coli | Absent/1 g | Absent | NT | NT | NT |

Abbreviations: NT = not tested; RRT = relative retention time
^{a} Total % Impurities is the sum of peaks ≥ 0.05%.

### Freeze-Thaw Study

A freeze-thaw study was performed using a FDV formulation of 10 mg/mL (concentration based as maralixibat chloride). The solution was cycled from −20° C. for 24 hours to room temperature for 5 hours, and samples were tested at the end of the fifth cycle. The results are provided in Table 19 and demonstrate that the FDV formulation is stable for up to five freeze-thaw cycles.

TABLE 19
Freeze Thaw Cycling Study Summary
for FDV Formulation, 10 mg/mL

| Attribute | Control, 2° C.-8° C. | After 5 Freeze-Thaw Cycles ^{a} |
|---|---|---|
| Appearance | Slightly | Slightly |
| | yellow liquid | yellow liquid^{b} |
| pH | 3.98 | 4.04 |
| Assay (% Label | 96.5 | 96.1 |
| Claim)^{c} | | |
| Assay (% Purity) | 97.4 | 97.4 |
| Impurities: Single | RRT 1.47 = | RRT 1.47 = |
| Largest Impurity | 0.27 | 0.27 |
| Total % | 0.56 | 0.56 |
| Impurities | | |

Abbreviations: RRT = relative retention time
^{a} Study lasted for five freeze cycles. Samples were cycled at −20° C. for 24 hours to room temperature for 5 hours. Samples were tested at the end of fifth cycle.
^{b}Appearance observed on the sample while at −20° C. in the cycle was an opaque, slightly yellow solid.
^{c}Expressed as maralixibat chloride.

### Conclusion for the Development of the FDV Formulation

Based on the results, the FDV formulation was established as presented in Table 20 and used for clinical studies. The MRX oral solution was originally prepared at Quotient Sciences on a patient specific basis based on their body weight and target dose. The required amount of maralixibat chloride was added to a clear borosilicate glass vial containing 30 mL of the grape-flavored diluent (Table 9) and mixed to form a clear solution. Visual confirmation of a clear solution was performed for each vial prior to administration. The vials containing the prepared solution was shipped under refrigerated conditions (2° C.-8° C.) to the clinical sites for administration according to the applicable study dosing instructions.

The FDV formulation was used in Phase 2 clinical studies, including the clinical studies for the proposed indication. The diluent for Phase 2 clinical supplies was manufactured at Formex (San Diego, Calif.) and the drug product was prepared at Quotient Sciences (located in United Kingdom).

TABLE 20
Composition of FDV Formulation

| Ingredients | Quality Standard | Function | Formulation^{a, b} |
|---|---|---|---|
| Maralixibat | In-House | MRX Drug | 0.2 to 50.0 mg/mL^{c} |
| Chloride | | Substance | |
| | | (API) | |
| Propylene | USP, Ph. Eur. | Preservative/ | 25.00% |
| Glycol | | Co-solvent | |
| Sucralose | NF, Ph. Eur. | Sweetener | 0.75% |
| Grape Flavor | In-House | Taste masking | 0.50% |
| | | agent | |
| Purified Water | USP, Ph. Eur. | Solvent | 73.75% |

| | |
|---|---|
| Total | 100.00% |

^{a}Amount of drug substance is determined based on the patient body weight and the dosing schedule per the applicable clinical study protocol.
^{b}The % w/w values are listed only for the components of the diluent vehicle.
^{c}Quantity is expressed as maralixibat chloride (salt form), which can be converted to maralixibat (free base) using a conversion factor of 0.95.

### Example 5: Fixed Drug Substance Concentration (FDSC) Formulation

For ongoing clinical studies and in preparation for the registration campaign (primary stability), multiple strengths (5 mg/mL, 10 mg/mL, 15 mg/mL, 20 mg/mL, 40 mg/mL and 50 mg/mL; concentrations based on maralixibat chloride) of ready-to-use MRX oral solutions were developed and manufactured at Unither; four (5 mg/mL, 10 mg/mL, 15 mg/mL, and 20 mg/mL; concentrations based on maralixibat chloride) were subsequently produced at larger scale at Halo. This ready-to-use MRX oral solution is a fixed drug substance concentration (FDSC) formulation that was developed based on the FDV formulation (Example 4).

As maralixibat is minimally absorbed and as the volumes of liquid formulation administered are small (≤3.0 mL per dose), the modification of excipients between FDV and the FDSC formulations is not expected to affect the bioavailability or efficacy. The commercial formulation was developed through minor adjustment to the composition amounts of the FDSC formulation in order to compensate for a slight bias to assay above target and to normalize the excipient levels across the formulation strengths under development.

### Propylene Glycol Level Adjustment

Propylene glycol has been recognized as an effective antimicrobial and antifungal agent in liquid and semi-solid preparations. As is the case for the FDV formulation (Example 4), this excipient serves dual functions in MRX oral solution: co-solvent and preservative. To assess the antimicrobial effectiveness (AET) of the FDV formulation, MRX oral solution, 5 mg/mL (concentration based on maralixibat chloride) was prepared at propylene glycol levels of 25 w/w, 30% w/w, and 35% w/w. The ALT study was performed in accordance with USP <51> and Ph. Eur. 5.1.3.

The results shown in Table 21 demonstrate that formulations with up to 30% w/w propylene glycol met the USP acceptance criteria but did not meet the Ph. Eur. acceptance criteria for the ALT test for oral solutions (Table 22). The USP and Ph. Eur. Acceptance criteria were both met only when the propylene glycol level was increased to 350% w/w. The amount of propylene glycol in the MRX oral solution was therefore adjusted from 250% in the FDV formulation to 3500 w/w.

TABLE 21
AET Testing Results for MRX Oral Solution
with Varied Levels of Propylene Glycol (PG)

| | Batch Tested | | | | | | | | |
|---|---|---|---|---|---|---|---|---|---|
| | 25% PG | | | 30% PG | | | 35% PG | | |
| | Log Reduction from | | | Log Reduction from | | | Log Reduction from | | |
| | Initial Inoculum | | | Initial Inoculum | | | Initial Inoculum | | |
| | 0 | 14 | 28 | 0 | 14 | 28 | 0 | 14 | 28 |
| Microorganism | hour | days | days | hour | days | days | hour | days | days |
| Aspergillus brasiliensis | 0.7 | 0.8 | 0.9 | 0.8 | 0.8 | 3.1 | NR | 2.0 | NI |
| Candida albicans | 4.0 | 4.0 | 4.0 | 4.0 | 4.0 | 4.0 | NR | 4.5 | NI |
| Escherichia coli | 1.6 | 4.9 | 4.9 | 1.6 | 4.9 | 4.9 | NR | 4.6 | NI |
| Pseudomonas aeruginosa | 4.8 | 4.8 | 4.8 | 4.8 | 4.8 | 4.8 | NR | 4.6 | NI |
| Staphylococcus aureus | 4.8 | 4.8 | 4.8 | 4.8 | 4.8 | 4.8 | NR | 4.4 | NI |

Abbreviations: NR = not reported; NI = no increase as defined by not more than
0.5 log unit higher than the value to which it is being compared.

TABLE 22
Acceptance Criteria for AET Testing

| Attributes | USP <51>^{a} | Ph. Eur. 5.1.3^{b} |
|---|---|---|
| Yeast and Molds: | No increase from the | Not less than1.0 log |
| Aspergillus brasiliensis | initial calculated | reduction from the |
| Candida albicans | count at 14 and | initial count at 14 |
| | 28 days | days, and no increase |
| | | from the 14 day's |
| | | count at 28 days |
| Bacteria: | Not less Than 1.0 log | Not less than 3.0 log |
| Escherichia coli | reduction from the | reduction form the |
| Pseudomonas aeruginosa | initial count at 14 | initial count at 14 |
| Staphylococcus aureus | days, and no increase | days, and no increase |
| | from the 14 days' | from the 14 day's |
| | count at 28 days | count at 28 days. |

^{a}For a category 3 product
^{b}For oral preparations

Although the stability results as summarized in Example 4 indicated that the FDV formulation has acceptable stability for clinical use, the level of an oxidation degradant, impurity desmethyl maralixibat chloride, increases over time in the FDV formulation. Desmethyl maralixibat chloride is an oxidation degradant that is also consistently observed in the drug substance synthesis, drug product stability and forced degradation studies.

During early process development, two types of mixing vessel for solution compounding, glass and stainless steel, were used and compared for their potential impact on product stability. Elevated desmethyl maralixibat chloride levels were observed when MRX oral solution (50 mg/ml) was compounded and stored in a glass or a stainless-steel vessel for up to 14 days under room temperature. However, solution stored in the stainless-steel vessel was found to have a higher desmethyl maralixibat chloride level compared to that stored in the glass jar (Table 23), suggesting the metal container potentially facilitates the oxidative degradation.

TABLE 23
Formation of desmethyl maralixibat chloride in a 50 mg/mL
MRX Oral Solution in Glass or Stainless-Steel Containers

| | | | |
|---|---|---|---|
| Initial | 3 Days | 7 days | 14 days |

| | |
|---|---|
| Sample | Results (% area)^{a} |

| | | | | |
|---|---|---|---|---|
| Glass Container | 0.08 | 0.14 | 0.20 | 0.26 |
| Stainless Steel | 0.15 | 0.59 | 0.79 | 1.10 |
| Container | | | | |

^{a}Two replicate measurements were performed at each timepoint.

Two replicate measurements were performed at each timepoint.

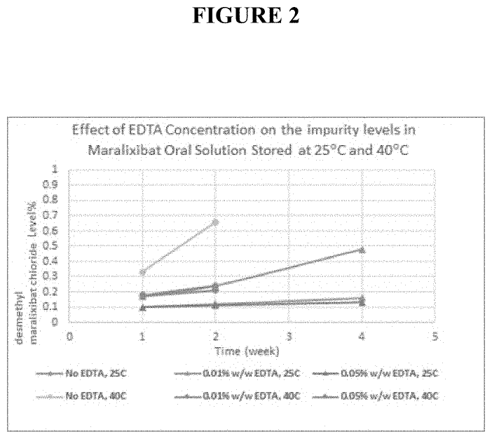

To inhibit drug oxidation, disodium ethylenediaminetetraacetic acid (EDTA) dihydrate was evaluated as a potential antioxidant in MRX oral solution. A laboratory scale stability study was performed in which disodium EDTA dihydrate was added to MRX oral solution (50 mg/mL, concentration based on maralixibat chloride) at levels of 0% w/w, 0.01% w/w, and 0.05% w/w and the level of the oxidation impurity (desmethyl maralixibat chloride) was monitored for up to 1 month at 25° C. and 40° C. Results of this study (FIG. 2) showed that the level of desmethyl maralixibat chloride in MRX oral solution is reduced with increasing disodium EDTA dihydrate concentrations. At a disodium EDTA dihydrate concentration of 0.05% w/w, no significant increase in the desmethyl maralixibat chloride level was observed after the solution was stored for 4 weeks at 25° C. and a slight increase after 2 weeks at 40° C. To further ensure the stability of MRX oral solution, disodium EDTA dihydrate at 0.1% w/w was selected for inclusion in the drug product.

To confirm that disodium edetate dihydrate is effective in inhibiting the degradation of maralixibat chloride when the solution is compounded in stainless steel vessels, a study was performed to compare the level of degradation product desmethyl maralixibat chloride in MRX oral solutions with or without disodium edetate dihydrate. Briefly, MRX oral solution with and without edetate was compounded in a stainless steel container and stirred at 30° C. for at least 2 hours as the worst-case scenario. The MRX oral solutions were packaged in 30 mL polyethylene terephthalate (PET) bottles with child resistant cap and induction sealed. The levels of desmethyl maralixibat chloride in the packaged drug products at 40° C./75% RH were monitored over time. The composition of the two solutions is provided in Table 24, and the results of the study is summarized in Table 25.

TABLE 24
Composition of MRX Oral Solution with
or without Disodium Edetate Dihydrate

| | | 20 mg/mL^{a}, no | 20 mg/mL^{a}, with |
|---|---|---|---|
| | | Disodium Edetate | Disodium Edetate |
| | | Dihydrate | Dihydrate |
| Ingredients | Function | % w/w | % w/w |
| Maralixibat | MRX Drug | 2.0 | 2.0 |
| Chloride^{a} | Substance | | |
| | (API) | | |
| Propylene | Co-solvent and | 35 | 35 |
| Glycol | Preservative | | |
| Purified Water^{b} | Solvent | 61.4 | 61.3 |
| Disodium Edetate | Antioxidant | — | 0.1 |
| Dihydrate | | | |
| Sucralose | Sweetener | 1.0 | 1.0 |
| Grape Flavor | Taste Masking | 0.5 | 0.5 |
| | Agent | | |

| | | |
|---|---|---|
| Total % | 99.9 | 99.9 |

^{a}Quantity is expressed as maralixibat chloride (salt form), which can be converted to maralixibat (free base) using a conversion factor of 0.95.
^{b}Amount of purified water is adjusted based on the assay of maralixibat chloride in order maintain the weight of 1.00 mL of the solution.

TABLE 25
Levels of desmethyl maralixibat chloride in MRX
Oral Solutions, 20 mg/mL^{a}, with or without Disodium
Edetate Dihydrate at 40° C./75% RH

| | Level of desmethyl maralixibat chloride % area | | |
|---|---|---|---|
| Ingredients | Initial | 1 month | 5 months |
| MRX Oral Solution | 0.69 | 2.52 | 23.15 |
| without Disodium | | | |
| Edetate Dihydrate | | | |
| MRX Oral Solution | 0.07 | 0.27 | 1.52 |
| with Disodium | | | |
| Edetate Dihydrate | | | |

^{a}Quantity is expressed as maralixibat chloride (salt form), which can be converted to maralixibat (free base) using a conversion factor of 0.95.

The study results show that the solution with disodium edetate dihydrate has a much a lower level of desmethyl maralixibat chloride oxidative impurity compared to the solution without this excipient (0.07% versus 0.69%) at time zero. When stored at the accelerated condition, the level of desmethyl maralixibat chloride in solution with disodium edetate dihydrate slowly increased from 0.07% to 1.52% over 5 months. However, in the solution without disodium edetate dihydrate, the level of desmethyl maralixibat chloride increased significantly from 0.69% to 23.15% in 5 months. In conclusion, addition of disodium edetate dihydrate at 0.1% w/w can effectively inhibit the degradation of MRX in the solution formulation even when a stainless steel vessel is used as compounding equipment.

### Composition for FDSC Formulation

Based on the results from the propylene glycol antimicrobial effect study and the disodium EDTA dihydrate antioxidant effect study, MRX oral solution formulation was optimized to establish the composition as shown in Table 26. MRX oral solution was manufactured as a ready-to-use, fixed drug substance concentration (FDSC) formulation that could be used directly.

In addition to the propylene glycol level increase and addition of disodium EDTA to the solution formulation, the level of sweetener (sucralose) was increased slightly (from 0.75% w/w to 1.0% w/w) in the FDSC formulation.

TABLE 26
Composition of FDSC Formulation

| | | | mg/mL | | | | | |
|---|---|---|---|---|---|---|---|---|
| | | | 5 | 10 | 15 | 20 | 40 | 50 |
| Ingredient | Grade | Function | mg/mL | mg/mL | mg/mL | mg/mL | mg/mL | mg/mL |
| Maralixibat | In-House | Active | 5.00 | 10.00 | 15.00 | 20.00 | 40.00 | 50.00 |
| Chloride^{a} | | Pharmaceutical | | | | | | |
| | | Ingredient | | | | | | |
| Propylene | USP/Ph. Eur | Co-solvent | 360.00 | 360.00 | 360.00 | 360.00 | 360.00 | 360.00 |
| Glycol | | and | | | | | | |
| | | Preservative | | | | | | |
| Purified | USP/Ph. Eur/JP | Solvent | 649.00 | 644.00 | 639.00 | 634.00 | 614.00 | 604.00 |
| Water ^{b} | | | | | | | | |
| Disodium | USP/Ph. Eur | Antioxidant | 1.00 | 1.00 | 1.00 | 1.00 | 1.00 | 1.00 |
| EDTA | | | | | | | | |
| dihydrate | | | | | | | | |
| Sucralose | NF/Ph. Eur | Sweetener | 10.00 | 10.00 | 10.00 | 10.00 | 10.00 | 10.00 |
| Grape | Non- | Taste Masking | 5.00 | 5.00 | 5.00 | 5.00 | 5.00 | 5.00 |
| Flavor | compendial | Agent | | | | | | |

| | | | | | | |
|---|---|---|---|---|---|---|
| Total (mg) | 1030.00 | 1030.00 | 1030.00 | 1030.00 | 1030.00 | 1030.00 |
| Total (mL) ^{c} | 1.00 | 1.00 | 1.00 | 1.00 | 1.00 | 1.00 |

^{a}The quantity of maralixibat chloride is adjusted based on assay value. The weight of drug substance is based on maralixibat chloride. The dose can be converted into maralixibat free base by equation: amount of maralixibat free base = amount of maralixibat chloride × 0.95
^{b} Amount of purified water to be used is adjusted based on the assay of maralixibat chloride and in order maintain the weight of 1.00 mL of the solution.
^{c} The weight (mg) of the unit formula is converted to volume (mL) using the density of the respective solution

TABLE 27
Comparison of FDV Formulation and FDSC Formulation

| | | | Quantity per Unit Dose (% w/w) | |
|---|---|---|---|---|
| | | | FDV | FDSC |
| Ingredient | Grade | Function | Formulation^{c} | Formulation |
| Maralixibat | In-House | MRX Drug | Variable | Fixed |
| Chloride ^{a} | | Substance | concentrations | concentration |
| | | (API) | up to 50 mg/mL | (5 to 50 mg/mL) |
| Propylene | USP | Co-solvent and | 25.00 | 32.03 to 35.00 |
| Glycol | Ph. Eur. | Preservative | | |
| Purified | USP | Solvent | 73.75 | 58.10 to 63.50 |
| Water ^{b} | Ph. Eur. | | | |
| Disodium EDTA | USP | Antioxidant | — | 0.1 |
| Dihydrate | Ph. Eur. | | | |
| Sucralose | NF | Sweetener | 0.75 | 0.92 to 1.00 |
| | Ph. Eur. | | | |
| Grape Flavor | In-House | Taste Masking | 0.50 | 0.46 to 0.50 |
| | | Agent | | |

| | | |
|---|---|---|
| Total % | 100 | 100 |

Abbreviations: API = active pharmaceutical ingredient
^{b} Quantity is expressed as maralixibat chloride (salt form), which can be converted to maralixibat (free base) using a conversion factor of 0.95.
^{c}Amount of purified water is adjusted based on the assay of maralixibat chloride in order maintain the weight of 1.00 mL of the solution.
^{d} The % w/w values are listed only for the components of the diluent vehicle.

### Conclusion for the Development of the FDSC Formulation

In conclusion, a ready-to-use FDSC formulation of MRX oral solution was developed based on the composition of the FDV formulation used in initial pediatric clinical studies, including the Phase 2 study for the proposed indication. Three compositional changes were made during the development:
1. Increasing propylene glycol level from 25% w/w to 35% w/w effectively improved antimicrobial effectiveness of the formulation.
2. Addition of disodium edetate dihydrate at a level of 0.1% w/w as antioxidant effectively inhibits the growth of degradant desmethyl maralixibat chloride.
3. Level of sucralose, a common sweetener, was increased from 0.75% w/w to 1% w/w.

The resulting FDSC formulation is demonstrated to be stable for long-term (e.g., 24-month) storage at 2° C.-8° C. and 25° C./60% RH at a wide concentration range. The bottle orientation and freeze-thaw cycles had no significant impact on the solution stability and the overall performance of the drug product.

### Example 6: Assessing the Efficacy of Combinational Therapy with ASBT Inhibitor and PPAR Agonists in a Preclinical Model of Sclerosing Cholangitis

Both inhibition of the intestinal bile acid transporter (IBAT), which blocks the enterohepatic circulation of bile acids (BA), and activation of the peroxisome proliferator-activated receptor (PPAR), which controls BA synthesis, conjugation, and transport have emerged as potential therapies for sclerosing cholangiopathies (SC), including PSC and PBC. Here we test the hypothesis that the combination of these treatment modalities increases efficacy over monotherapies in the MDR2−/− mouse model of SC.

Methods: 30-day-old female MDR2−/− mice (FVB background) were treated daily for 14 days by orogastric gavage with either vehicle control (Kolliphor and CMC), 100 mg/kg/day bezafibrate (pan-PPAR agonist), 100 mg/kg/day fenofibrate (PPARα agonist), 10 mg/kg seladelpar (PPARδ agonist), 0.008% SC-435 (non-absorbable IBAT inhibitor) admixed to chow, or a combination of SC-435 and PPAR agonists.

Results: Compared with wildtype (WT) mice, the liver to body weight ratio was nearly doubled in MDR2−/− mice which was not reduced with PPAR agonist monotherapies but attenuated with IBATi and combination therapy. Liver and serum BA and biochemistries were highly elevated in vehicle treated MDR2−/− mice (mean±SE for liver BA: 930±84 nmol/g, serum BA: 336±40 μM, ALT: 1275±47 IU/L, total bilirubin [TB]: 2.0±0.4 mg/dL, ALP: 296±17 IU/L) compared with WT (Figure). PPAR agonists and IBATi significantly reduced retention of BA in the liver, but only fenofibrate and IBATi alone or in combination with PPAR agonists decreased serum BA concentrations. All treatments except for fenofibrate reduced serum ALT levels. While IBATi treatment reduced serum TB concentrations, monotherapies with PPAR agonists did not. In contrast to studies in other mouse backgrounds, serum ALP was increased by IBATi treatment alone and further raised by combination with fibrates in this FVB mouse background. ALP was not increased with combination of IBATi and PPARδ agonist. Serum ALP levels correlated with biliary mass and bile duct proliferation, as assessed by CK19 immunohistochemistry.

Conclusion: IBATi is more potent than PPAR agonists in reducing serum total BA and TB levels, markers of cholestasis. The combination therapy of IBATi and PPARδ agonist shows a synergistic effect in this mouse model of SC. Further preclinical investigations may help to better understand the mechanisms underlying the synergy and potential adverse effects and to guide use in clinical trials.

As various changes can be made in the above-described subject matter without departing from the scope and spirit of the present invention, it is intended that all subject matter contained in the above description, or defined in the appended claims, be interpreted as descriptive and illustrative of the present invention. Many modifications and variations of the present invention are possible in light of the above teachings. Accordingly, the present description is intended to embrace all such alternatives, modifications, and variances which fall within the scope of the appended claims.

All patents, applications, publications, test methods, literature, and other materials cited herein are hereby incorporated by reference in their entirety as if physically present in this specification.

1. A pharmaceutical composition comprising an ASBTI, a preservative, and an antioxidant, wherein the ASBTI is 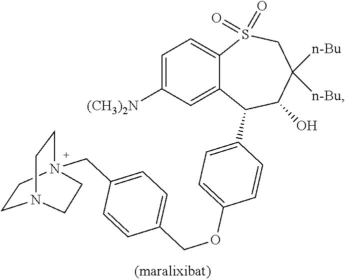
or a pharmaceutically acceptable salt thereof.

2. (canceled)

3. The pharmaceutical composition of claim 1, wherein the ASBTI is

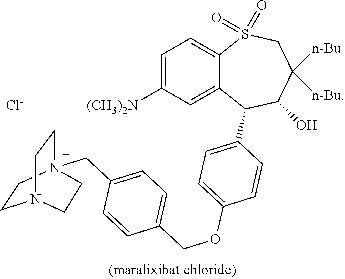

4.-7. (canceled)

8. The pharmaceutical composition of claim 1 wherein the ASBTI is present in an amount of about 0.1 mg/mL to about 500 mg/mL of the composition.

9.-12. (canceled)

13. The pharmaceutical composition of claim 1 wherein the ASBTI is present in an amount of about 9 mg/mL to about 10 mg/mL of the composition.

14. The pharmaceutical composition of claim 1 wherein the preservative is an antimicrobial preservative selected from the group consisting of propylene glycol, ethyl alcohol, glycerin, benzalkonium chloride, benzethonium chloride, benzoic acid, benzyl alcohol, butylparaben, cetrimide (cetyltrimethylammonium bromide), cetrimonium bromide, cetylpyridinium chloride, chlorhexidine, chlorobutanol, chlorocresol, cresol, ethylparaben, methylparaben, phenol, phenoxyethanol, phenylethyl alcohol, phenylmercuric acetate, phenylmercuric borate, phenylmercuric nitrate, propylparaben, sodium benzoate, sodium dehydroacetate, sodium propionate, sorbic acid, potassium sorbate, thimerosal, thymol, and combinations thereof.

15. (canceled)

16. The pharmaceutical composition of claim 1, wherein the preservative is propylene glycol.

17. (canceled)

18. The pharmaceutical composition of claim 1, wherein the preservative is present in an amount of from about 30% to about 40% of the composition.

19.-23. (canceled)

24. The pharmaceutical composition of claim 1, wherein the antioxidant is selected from the group consisting of an aminocarboxylic acid, an aminopolycarboxylic acid, ascorbic acid, ascorbyl palmitate, butylated hydroxyanisole, butylated hydroxytoluene, monothioglycerol, sodium ascorbate, sodium formaldehyde sulfoxylate, sodium metabisulfite, BHT, BHA, sodium bisulfite, vitamin E or a derivative thereof, propyl gallate, and combinations thereof.

25. The pharmaceutical composition of claim 1, wherein the antioxidant is an aminopolycarboxylic acid selected from EDTA (ethylenediaminetetraacetic acid), DTPA (diethylenetriaminepentaacetic acid), EGTA (ethylene glycol-bis(β-aminoethyl ether)-N,N,N′,N′-tetraacetic acid), NTA (nitrilotriacetic acid), BAPTA (1,2-bis(o-aminophenoxy)ethane-N,N,N′,N′-tetraacetic acid), NOTA (2,2′,2″-(1,4,7-triazonane-1,4,7-triyl)triacetic acid), DOTA (tetracarboxylic acid), and EDDHA (ethylenediamine-N,N′-bis(2-hydroxyphenylacetic acid).

26. The pharmaceutical composition of claim 1, wherein the antioxidant is EDTA.

27. The pharmaceutical composition of claim 1, wherein the antioxidant is present in an amount of about 0.001% to about 1% w/w of the composition.

28.-31. (canceled)

32. The pharmaceutical composition of claim 1, wherein the antioxidant is present in an amount of about 0.10% w/w of the composition.

33. The pharmaceutical composition of claim 1, wherein the composition is stable for at least 1 month at room temperature.

34.-38. (canceled)

39. The pharmaceutical composition of claim 1, wherein the composition is a liquid composition for oral administration.

40. The pharmaceutical composition of claim 39, wherein the composition is an aqueous solution.

41. The pharmaceutical composition of claim 1, further comprising a sweetener, a taste-masking ingredient, or a combination thereof.

42. A pharmaceutical composition comprising:
a. from about 5 mg/mL to about 50 mg/mL of maralixibat;
b. from about 300 mg/mL to about 400 mg/mL of propylene glycol;
c. about 1 mg/mL of disodium EDTA;
d. a sweetener, a taste-masking ingredient, or a combination thereof, and
e. water.

43. The pharmaceutical composition of claim 42, comprising:
a. from about 8 mg/mL to about 20 mg/mL of maralixibat;
b. from about 330 mg/mL to about 380 mg/mL of propylene glycol;
c. about 1 mg/mL of disodium EDTA;
d. a sweetener, a taste-masking ingredient, or a combination thereof, and
e. water.

44. (canceled)

45. The pharmaceutical composition of claim 1, further comprising a second therapeutic agent.

46. The pharmaceutical composition of claim 44, wherein the second therapeutic agent is ursodeoxycholic acid (UDCA), rifampicin, an antihistamine, or an FXR-targeting drug.

47. A pharmaceutical dosage form for oral administration comprising the pharmaceutical composition of claim 1.

48. A method of treating or ameliorating a pediatric cholestatic liver disease comprising administering to a pediatric subject a therapeutically effective amount of the pharmaceutical composition of claim 1.

49. The method of claim 48, wherein the pediatric cholestatic liver disease is progressive familial intrahepatic cholestasis (PFIC), PFIC type 1, PFIC type 2, PFIC type 3, Alagille syndrome (ALGS), biliary atresia (BA), post-Kasai biliary atresia, post-liver transplantation biliary atresia, Dubin-Johnson Syndrome, post-liver transplantation cholestasis, post-liver transplantation associated liver disease, intestinal failure associated liver disease, bile acid mediated liver injury, pediatric primary sclerosing cholangitis (PSC), MRP2 deficiency syndrome, neonatal sclerosing cholangitis, a pediatric obstructive cholestasis, a pediatric non-obstructive cholestasis, a pediatric extrahepatic cholestasis, a pediatric intrahepatic cholestasis, a pediatric primary intrahepatic cholestasis, a pediatric secondary intrahepatic cholestasis, benign recurrent intrahepatic cholestasis (BRIC), BRIC type 1, BRIC type 2, BRIC type 3, total parenteral nutrition associated cholestasis, paraneoplastic cholestasis, Stauffer syndrome, drug-associated cholestasis, infection-associated cholestasis, or gallstone disease.

50. (canceled)

51. (canceled)

52. A method of treating or ameliorating pruritus comprising administering to a pediatric subject a therapeutically effective amount of the pharmaceutical composition of claim 1.

53. A method of treating or ameliorating hypercholemia comprising administering to a pediatric subject a therapeutically effective amount of the pharmaceutical composition of claim 1.

54. A method of treating or ameliorating xanthoma comprising administering to a pediatric subject a therapeutically effective amount of the pharmaceutical composition of claim 1.

55. A method of decreasing the level of serum or hepatic bile levels in a subject comprising administering to a pediatric subject a therapeutically effective amount of the pharmaceutical composition of claim 1.

56.-59. (canceled)

60. A method of treating or ameliorating a pediatric cholestatic liver disease comprising administering to a pediatric subject a therapeutically effective amount of the pharmaceutical composition of claim 1 in combination with a subclinical therapeutically effective amount of a second therapeutic agent selected from the group consisting of UDCA, rifampicin, an antihistamine, an FXR-targeting drug, and a PPAR agonist, wherein the subclinical therapeutically effective amount of the second therapeutic agent is at least 10% lower than the amount of the second therapeutic agent administered as a monotherapy.

61.-68. (canceled)

69. A method of treating or ameliorating a pediatric cholestatic liver disease comprising administering to a pediatric subject a therapeutically effective amount of maralixibat in combination with a therapeutically effective amount of a PPAR agonist.

70. The method of claim 69, wherein the PPAR agonist is selected from bezafibrate, seladelpar (MBX-8025), GW501516 (Cardarine), fenofibrate, elafibranor, REN001, KD3010, ASP0367, and CER-002.

71.-74. (canceled)
